(19) United States
(12) Patent Application Publication
(10) Pub. No.: US 2014/0242690 A1
(43) Pub. Date: Aug. 28, 2014

(54) CYSTIC FIBROSIS TREATMENT

(71) Applicants: THE CHANCELLOR MASTERS AND SCHOLARS OF THE UNIVERSITY OF OXFORD, Oxford (GB); THE UNIVERSITY COURT OF THE UNIVERSITY OF EDINBURGH, Edinburgh (GB); IMPERIAL INNOVATIONS LTD, London (GB)

(72) Inventors: Eric Walter Frederick Wolfgang Alton, London (GB); Jane Carolyn Davies, London (GB); Uta Griesenbach, Surrey (GB); Stephen Hyde, Oxford (GB); Deborah Gill, Oxford (GB); Lee Davies, Appleton (GB); David John Porteous, Edinburgh (GB); Alan Christopher Boyd, Edinburgh (GB); Alastair Innes, Edinburgh (GB)

(21) Appl. No.: 14/354,501

(22) PCT Filed: Oct. 29, 2012
(86) PCT No.: PCT/GB2012/052685

§ 371 Date: Apr. 25, 2014

(30) Foreign Application Priority Data
Oct. 28, 2011 (GB) 1118704.4

(51) Int. Cl. C07K 14/705

(52) U.S. Cl. CPC C07K 14/705
USPC 435/320.1

(57) ABSTRACT

The present invention relates to medicaments for the treatment of cystic fibrosis. Also provided are methods of preparing such medicaments so that they can be used in a treatment regime for cystic fibrosis. The present invention also provides a kit of parts including the medicament of the invention, as well as treatment regimes for cystic fibrosis.

The present invention concerns medicaments for the treatment of cystic fibrosis, methods of preparing such medicaments, and treatment regimens.

Cystic fibrosis is an inherited disease of the secretory glands which mainly affects the lungs, pancreas and liver. Currently there is no cure. Cystic fibrosis is by caused a mutation in the gene coding for the cystic fibrosis transmembrane conductance regulator (CFTR) polypeptide. CFTR polypeptide is an ion channel that transports chloride and thiocyanate across the epithelial cell membrane and hence acts to regulate components of sweat, digestive juices and mucus. Mutations of the CFTR gene affect functioning of the chloride ion channels in these cell membranes, causing an imbalance in ion and fluid transport. It is thought that this causes the production of abnormally dehydrated and thick mucus in the lungs and pancreas which leads to pancreatic disorders and lung infections.

In the UK, over 9000 individuals have cystic fibrosis and it is thought that over 2 million people carry a mutation in the CFTR gene. Respiratory dysfunction is the most serious symptom and results from frequent lung infections. Most individuals die in their 20 s and 30 s from lung failure and lung transplantation is often necessary as CF worsens. A multitude of other symptoms, including sinus infections, poor growth, diarrhoea and infertility result from the effects of CF on other parts of the body.

Cystic fibrosis is a recessive genetic disorder. Sequence analysis of the CFTR gene of CF chromosomes has revealed a variety of disease causing mutations. To date, >1000 disease causing mutations in the CFTR gene have been identified. The most common mutation, comprising over 60% of all mutant alleles, results in the deletion of phenylalanine in the ATP binding cassette at position 508 of the CFTR protein. The resultant AF508 CFTR polypeptide is expressed as a large, misfolded nascent polypeptide which is prematurely destroyed via the ubiquitin pathway but which aggregates following defective processing in the translocation machinery.

In recent years a number of attempts have been made to treat CF using gene therapy. Gene therapy is the insertion, alteration, or removal of genes within an individual's cells and biological tissues to treat disease. It is a technique for correcting defective genes that are responsible for disease development. The most common form of gene therapy involves the insertion of functional genes into an unspecified genomic location in order to replace a mutated gene, but other forms involve directly correcting the mutation or modifying normal gene that enables a viral infection. Although the technology is still in its infancy, it has been used with some success.

As a general guide, gene therapy can be applied using viral or non-viral techniques. Non-viral methods can present certain advantages over viral methods, with simple large scale production and low host immunogenicity being just two. To improve the delivery of the non-viral DNA into the cell, the DNA can be protected from damage. Cationic lipids, due to their positive charge, were first used to condense negatively charged DNA molecules so as to facilitate the encapsulation of DNA into liposomes. Later it was found that the use of cationic lipids significantly enhanced the stability of lipoplexes. Also as a result of their charge, cationic liposomes interact with the cell membrane, endocytosis was widely believed as the major route by which cells uptake lipoplexes.

CF is a good candidate for gene therapy as it is primarily caused by mutations in a single gene. A normal copy of the gene could be delivered to patients via topical delivery to the lung, not requiring invasive techniques or surgery. A gene complementation approach would also directly target the cause of the disease and could correct many aspects of the complex lung pathology. A single therapy to treat the underlying defect could greatly reduce the high therapeutic burden that CF patients currently have to endure. In addition, one therapy might be suitable to treat subjects with a wide variety of mutations in the CFTR gene, meaning that a single treatment strategy would be relevant to all patients.

Soon after the cloning of the CFTR gene, proof-of-principle was established when the Cl-conductance defect was corrected after delivery of a functional copy of human wild-type CFTR DNA to cells isolated from CF patients. To date, a number of trials for CF gene therapy have been tested in humans. These early studies were concerned mainly with safety issues.

There is at present no cure for CF. Current treatments are merely palliative: they include antibiotic treatment (for lung infections), chest physiotherapy/mechanical expectoration (for mucus accumulation), surgery and mechanical ventilation.

Hence clearly there remains a need for the development of new therapies for the treatment of cystic fibrosis.

A first aspect of the invention provides a pharmaceutical composition comprising from 1 ml to less than 10 mls of a complex of (i) a non-viral CpG dinucleotide-free plasmid comprising nucleic acid encoding a CFTR polypeptide operatively linked to hCEF1 promoter, wherein the plasmid is at a concentration of 2 mg/ml to 3 mg/ml, and (ii) GL67A lipid mixture at a concentration of 10 mg/ml to 20 mg/ml.

It can be appreciated that it is desirable to identify an efficacious amount of a pharmaceutical composition that can be administered to a patient without provoking unacceptable side effects, which may not only cause discomfort to the patient, but can also affect the effect of the performance of the composition. The present inventors therefore decided to investigate a range of amounts of pharmaceutical composition used in treatment regimens to identify a suitable quantity of pharmaceutical composition and preferred means of administration.

By “from 1 ml to less than 10 mls” preferably the composition comprises at least 1 ml up to but not including 10 mls, as well as all amounts within that range; for example 1 ml, 2 mls, 3 mls, 4 mls, 5 mls, 6 mls, 7 mls, 8 mls, 9 mls and to but not including 10 mls.

As can be seen in the accompanying examples, the inventors performed a series of trials with the pharmaceutical composition of this aspect of the invention. While unit doses of 10 mls and 20 mls, containing 2 mg/ml to 3 mg/ml of the non-viral CpG dinucleotide-free plasmid and 10 mg/ml to 20 mg/ml GL67A lipid mixture, caused unacceptable side-effects in the subjects, surprisingly doses of from 1 ml to less than 10 mls, in particular 5 mls, were tolerated with no apparent side-effects. Moreover, it can be seen that the results of the trials demonstrated that this unit dose of pharmaceutical composition showed statistically significant evidence of a positive treatment effect.

Accordingly a preferred embodiment of the first aspect of the invention is wherein the composition comprises 5 mls of the non-viral CpG dinucleotide-free plasmid/GL67A lipid mixture complex.

The data provided herein demonstrates that the inventors have identified a preferred quantity of pharmaceutical composition which offers great promise when used for the treatment of cystic fibrosis. It has hitherto not been appreciated that such a quantity of the pharmaceutical composition would be preferable.

The pharmaceutical composition of this aspect of the invention contains 2 mg/ml to 3 mg/ml of the non-viral CpG dinucleotide-free plasmid. The concentration of plasmid used within the composition can be varied within this range. Preferably the concentration ranges between 2.28 mg/ml to 2.8 mg/ml, and values within this range are included in the aspect of the invention. For example, the concentration of plasmid used within the composition can be 2.3 mg/ml, 2.35 mg/ml, 2.4 mg/ml, 2.45 mg/ml, 2.5 mg/ml, 2.55 mg/ml, 2.6 mg/ml or 2.65 mg/ml, 2.7 mg/ml, 2.75 mg/ml and 2.8 mg/ml. Preferably the concentration of plasmid used within the composition is 2.65 mg/ml.

The pharmaceutical composition of this aspect of the invention contains 10 mg/ml to 20 mg/ml GL67A lipid mixture. The concentration of the GL67A lipid mixture can be varied within this range, however it is preferred that the GL67A lipid mixture is at a concentration of 12.2 mg/ml to 16.4 mg/ml and values within this range are included in the aspect of the invention. For example, the concentration of the GL67A lipid mixture used within the composition can 12.5 mg/ml, 13 mg/ml. 13.5 mg/ml, 14 mg/ml, 14.5 mg/ml, 15 mg/ml, 15.5 mg/ml, 16 mg/ml or 16.4 mg/ml. Preferably the concentration of the GL67A lipid mixture used within the composition is in the range of 14 to 14.5 mg/ml, for example, 14.1 mg/ml, 14.2 mg/ml, 14.3 mg/ml, 14.4 mg/ml or 14.5 mg/ml. More preferably the concentration is between 14.25 to 14.35 mg/ml, for example 14.26 mg/ml, 14.27 mg/ml, 14.28 mg/ml, 14.29 mg/ml, 14.3 mg/ml, 14.31 mg/ml, 14.32 mg/ml, 14.33 mg/ml, 14.34 mg/ml or 14.35 mg/ml. Preferably the concentration of GL67A lipid mixture used within the composition is 14.31 mg/ml.

Accordingly a preferred embodiment of the first aspect of the invention is wherein the non-viral CpG dinucleotide-free plasmid is at a concentration of 2.28 mg/ml to 2.8 mg/ml and (ii) the GL67A lipid mixture is at a concentration of 12.2 mg/ml to 16.4 mg/ml.

More preferably the pharmaceutical composition comprises from 1 ml to less than 10 mls, preferably 5 mls, of a complex of (i) a non-viral CpG dinucleotide-free plasmid comprising nucleic acid encoding a CFTR polypeptide operatively linked to hCEF1 promoter, wherein the plasmid is at a concentration of 2.65 mg/ml, and (ii) GL67A lipid mixture at a concentration of 14.31 mg/ml.

The present inventors have investigated the effectiveness of the pharmaceutical composition of the first aspect of the invention as a medicament comprising a “gene therapy product”. As used herein, both “medicament” and “gene therapy product” relates to a complex of (i) a non-viral CpG dinucleotide-free plasmid comprising nucleic acid sequence encoding a cystic fibrosis transmembrane conductance regulator (CFTR) polypeptide operatively linked to hCEFI promoter and (ii) GL67A lipid mixture.

The pharmaceutical composition of the first aspect of the invention comprises a non-viral CpG dinucleotide-free plasmid comprising nucleic acid sequence encoding a cystic fibrosis transmembrane conductance regulator (CFTR) polypeptide operatively linked to hCEFI promoter.

Non-viral plasmids are capable of growing in appropriate host cells, preferably E. coli and of expressing nucleic acid sequences within the plasmid in the desired subject, preferably humans.

Non-viral plasmids cannot replicate in the subject to be treated, as they lack the viral genetic material which hijacks the body's normal production machinery. However they are capable of replicating in appropriate host cells, such as yeasts or bacteria including E. coli.

The term “plasmid” as used herein refers to a construction comprised of genetic material designed to direct transformation of a targeted cell. The plasmid contains a plasmid backbone. A “plasmid backbone” as used herein contains multiple genetic elements positionally and sequentially oriented with other necessary genetic elements such that the nucleic acid in a nucleic acid cassette can be transcribed and when necessary translated in the transfected cells.

The plasmid backbone can contain one or more unique restriction sites within the backbone. The plasmid may be capable of autonomous replication in a defined host or organism such that the cloned sequence is reproduced. The plasmid can confer some well-defined phenotype on the host organism which is either selectable or readily detected. The plasmid or plasmid backbone may have a linear or circular configuration. The components of a plasmid can contain, but is not limited to, a DNA molecule incorporating: (1) DNA; (2) the plasmid backbone; (3) a sequence encoding a therapeutic or desired product; and (4) regulatory elements for transcription, translation, RNA stability and replication

The purpose of the plasmid in human gene therapy for the efficient delivery of nucleic acid sequences to, and expression of therapeutic genes in, a cell or tissue. In particular, the purpose of the plasmid is to achieve high copy number, avoid potential causes of plasmid instability and provide a means for plasmid selection. As for expression, the nucleic acid cassette contains the necessary elements for expression of the nucleic acid within the cassette. Expression includes the efficient transcription of an inserted gene, nucleic acid sequence, or nucleic acid cassette with the plasmid. For the present invention, the expression product is the CFTR polypeptide.

The non-viral plasmid used in the pharmaceutical composition of the first aspect of the invention is a “CpG dinucleotide-free plasmid”.

The presence of CpG dinculeotides can generate flu like symptoms and inflammation, particularly when administered in the airway. The elimination of CpG dinucleotides can help eliminate such effects. The inflammatory response observed after plasmid/liposome complex delivery arises in part from the recognition of the unmethylated CpG dinucleotides present in the bacterially derived pDNA. Mammalian DNA differs from bacterial DNA in that the frequency of CpG dinucleotides is severely suppressed compared to that of bacterial DNA and most mammalian CpG sequences are methylated. Bacterially derived plasmid DNA activates several immune/inflammatory cell types, including B cells, macrophages, dendritic cells, and natural killer cells. The inventors have previously determined that the presence of a single CpG dinucleotide can lead to an inflammatory response.

Several strategies could be employed to decrease the immunostimulatory properties of constructs. One approach might be to enzymatically methylate all CpG sequences. While the in vitro methylation of all the CpG dinucleotides within a given pDNA significantly decreases inflammatory consequences of plasmid/liposome delivery to the lung, it also severely inhibits transgene expression. Thus, although methylation may be employed in a preferred instance it is not. An alternative approach which may be employed is to eliminate or reduce the frequency of CpG sequences in the plasmids used in the invention. This may, for instance, be done by eliminating nonessential regions within the construct (e.g., sequences flanking the origin of replication) and also, for instance, by redesigning regulatory elements and open reading frames to remove CpG sequences.

Thus, for instance, plasmids employed in the invention have been modified to eliminate CpG dinucleotides from the naturally occurring sequence. The lack of CpG dinculeotide content helps minimise inflammatory responses induced by the vector.

The non-viral CpG dinucleotide-free plasmid used in the medicament comprises nucleic acid sequence encoding a cystic fibrosis transmembrane conductance regulator (CFTR) polypeptide.

The CFTR gene is approximately 189 Kb in length and in humans is located on chromosome 7. The CFTR cDNA includes the open reading frame (ORF) encoding the 1480 amino acids of the CFTR polypeptide, along with 5′ UTR and 3′ UTR sequences. Examples of nucleic acid sequences encoding the CFTR polypeptide are well known and can be readily located by the skilled person with no inventive requirement. For example, GenBank (http://www.cbni.nlm.nih.gov/nuccore) provides a searchable database of nucleic acid sequences, which includes examples of CFTR-encoding polynucleotides (e.g. see (http://www.cbni.nlm.nih.gov/gene/1080).

As discussed above, the non-viral CpG dinucleotide-free plasmid has been modified to remove CpG dinucleotides. The native CFTR gene and CFTR cDNA sequences contain a number of CpG dinucleotides. Those dinucleotides can be removed from the CFTR nucleic acid sequences provided above using standard molecular biology techniques for nucleotide replacement; for example, see Sambrook et al (2001) Molecular Cloning: A Laboratory Manual, CSHL Press. It is important to retain the amino acid sequence of the full length wild type CFTR protein. An example of a nucleic acid sequence encoding CFTR is provided as part of SEQ ID NO:1 (nucleotides 738-5310, listed at the end of the description) and further discussed below in relation to the plasmid pGM169.

The non-viral CpG dinucleotide-free plasmid used in the pharmaceutical composition of the present invention also comprises a hCEFI promoter.

The hCEFI promoter is a composite of a human CMV enhancer operably linked to a human EFIa promoter. It has been previously demonstrated that the hCEFI promoter gives rise to prolonged and high-level expression in human cells of nucleic acid sequences when arranged as an ‘expression cassette’ in a non-viral plasmid.

Examples of nucleic acid sequences for the human CMV enhancer can be readily located by the skilled person with no inventive requirement. For example, GenBank (http://www.cbni.nlm.nih.gov/nuccore) provides a searchable database of nucleic acid sequences, which includes examples of human CMV enhancer; e.g. the region from 175182 to 174879 of NCBI Accession No: BK000394.1.

Examples of nucleic acid sequences for the human EFIa promoter can be readily located by the skilled person with no inventive requirement. For example, GenBank (http://www.cbni.nlm.nih.gov/nuccore) provides a searchable database of nucleic acid sequences, which includes examples of human EFIa promoter; e.g. the region from nucleotides 13-231 (NCBI Accession No: EF362804.1) in the human elongation factor 1 alpha gene.

As discussed above, the non-viral CpG dinucleotide-free plasmid has been modified to remove CpG dinucleotides. The native human CMV enhancer and human EFIa promoter contain a number of CpG dinucleotides. Those dinucleotides can be removed from the hCEFI promoter nucleic acid sequences using standard molecular biology techniques for nucleotide replacement; for example, see Sambrook et al (2001) Molecular Cloning: A Laboratory Manual, CSHL Press. An example of a nucleic acid sequence of the hCEFI promoter is provided as part of SEQ ID NO:1 (nucleotides 7-538, listed at the end of the description) and further discussed below in relation to the plasmid pGM169.

“Operably linked” refers to an arrangement of elements wherein the components so described are configured so as to perform their usual function. Thus, a promoter operably linked to a nucleic acid sequence is capable of effecting the expression of that sequence when the proper enzymes are present. The hCEFI promoter need not be contiguous with the sequence, so long as it functions to direct the expression thereof. Thus, the sequence to be expressed will be transcribed due to the hCEFI promoter. In a preferred instance any of the components described herein will be in operable linkage when present in a non-viral CpG dinucleotide-free plasmid present in the medicament of the first aspect of the invention.

In a preferred embodiment of the first aspect of the invention, the non-viral CpG dinucleotide-free plasmid has a nucleic acid sequence at least 90% identical to the nucleic acid sequence provided in SEQ ID NO:1.

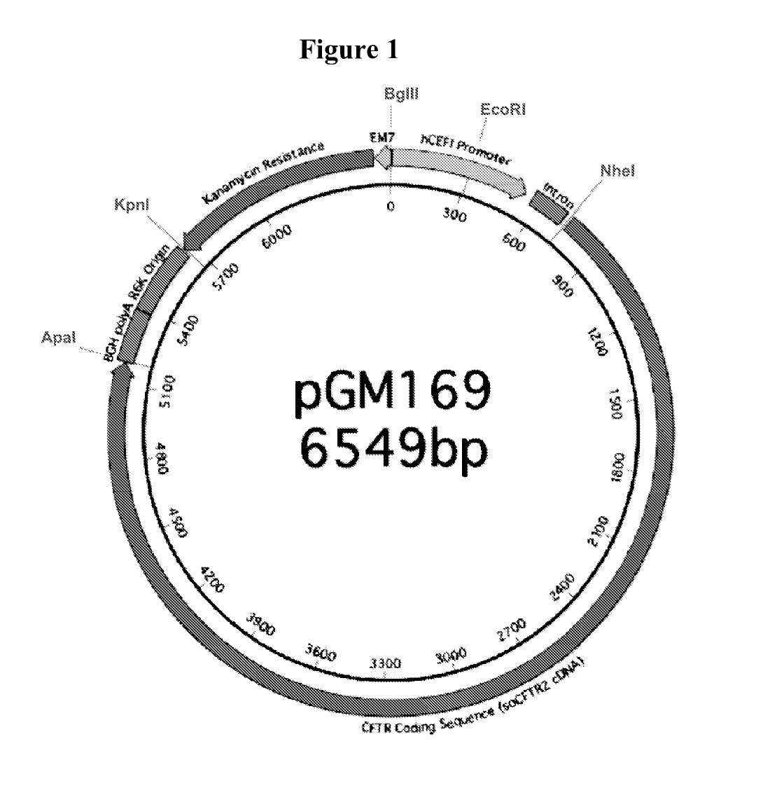

Plasmid pGM169 is a covalently closed circular double-stranded plasmid DNA molecule of 6549 base pairs purified from bacteria. It is based on a CpG-free plasmid backbone. A full sequence listing is presented at the end of the description. A diagrammatic representation of pGM169 is shown in FIG. 1.

The basic features of pGM169 (proceeding clockwise from Obp) are: the CpG-free hCEFI enhancer/promoter; a CpG-free synthetic intron sequence to enhance mRNA splicing; a CpG-free version of the CFTR coding sequence termed soCFTR2; a CpG-free version of the bovine growth hormone polyadenylation sequence; a CpG-free version of the R6K bacterial plasmid origin of replication; a CpG-free version of the kanamycin resistance gene, and a CpG-free synthetic bacterial promoter sequence termed EM7. pGM169 was largely constructed by the molecular ligation of synthetic DNA fragments created by de novo chemical synthesis undertaken by GeneArt AG. (Regensburg, Germany). Convenient restriction enzyme sites (indicated on the diagram in FIG. 1) were placed between the sequence elements to facilitate final assembly. The one exception to this synthesis strategy was the soCFTR2 cDNA. A cDNA termed soCFTR was completely assembled by de novo chemical synthesis, but was found to contain three nucleotide deviations from the preferred sequence. These were corrected in soCFTR2 by a combination of de novo chemical synthesis and PCR-directed mutagenesis.

Sequence Elements Within pGM169
hCEFI Enhancer/Promoter

The hCEFI enhancer/promoter consists of a CpG-free version of the immediate early enhancer from human CMV fused to a CpG-free version of the human elongation factor 1 alpha promoter. The CpG-free form of the human CMV enhancer spans the region from nucleotides 7-308 in pGM169. This region is highly homologous to nucleotides 175182-174879 (NCBI Accession No: BK000394.1) in human herpesvirus 5 strain AD169 except that all CG dinucleotides have been substituted for TG dinucleotides. The CpG-free form of the elongation factor 1 alpha promoter spans nucleotides 315-538 in pGM169. This sequence largely corresponds to nucleotides 13-231 (NCBI Accession No: EF362804.1) in the human elongation factor 1 alpha gene except that all CG dinucleotides have been substituted for alternative dinucleotides. The TATA box within the CpG-free form of the elongation factor 1 alpha promoter is found at pGM169 nucleotides 508-514. The pGM169 CFTR mRNA is predicted to start at nucleotide 539, which is the transcriptional start site of the human elongation factor 1 alpha promoter. The selection of hCEFI enhancer/promoter was based on a systematic analysis of CpG-free enhancers and promoters. Importantly, the hCEFI enhancer/promoter directs persistent expression of reporter gene and CFTR cDNA sequences following aerosol delivery with GL67A to the lungs of mice.

### Non-Coding Sequences

Positioned between the hCEFI enhancer/promoter and the CFTR coding sequence, at nucleotides 539-737 in pGM169, is a region of transcribed noncoding sequence consisting of two synthetic CpG-free exons and a synthetic CpG-free intron. The role of these 5′ non-coding sequences is to enhance the mRNA processing and translation of the adjacent CFTR coding sequence. These 5′ non-coding sequences lack significant homology to any human sequences in the NCBI database at the time of drafting this patent application. The intron donor site (G|GT) is located at nucleotides 569-571 and the intron acceptor site (AG|G) is located at nucleotides 708-710, and the length of the intron is 140 nucleotides. At the 3′ end of the 5′ non-coding sequences, at nucleotides 733-737 in pGM169, is a consensus Kozak sequence (CCACC) that facilitates efficient translation of the adjacent CFTR coding sequence.

### CFTR Coding Sequence

The CFTR cDNA sequence recorded at the NCBI DNA database has been revised three times since the sequence was originally described in 1989. The original CFTR cDNA sequence (NCBI Accession No: M28668.1) contained a number of sequencing errors and a polymorphic region that was later identified to be rare in the general population. NCBI updated the CFTR cDNA database entry to reflect these errors in 1999 (NCBI Accession No: NM_{—}000492.1). Subsequently, NCBI curators mistakenly reintroduced the original sequencing errors and rare polymorphic region in an updated CFTR cDNA database entry in 2000 (NCBI Accession No: NM_{—}000492.2). Following the submission of a large amount of human genome sequencing data from the University of Washington (Seattle, USA) the NCBI further amended the CFTR cDNA database entry in 2006 (NCBI Accession No: NM_{—}000492.3). The deduced protein sequence from these various NCBI database entries differ at CFTR amino acids 470, 620 and 833. These differences, and the amino acids encoded by pCF1-CFTR and pGM169 are detailed in Table 1.

TABLE 1
CFTR sequence variation at amino acids 470, 620 and 833

| | CFTR Amino acid | | |
|---|---|---|---|
| CFTR sequence | 470 | 620 | 833 |
| M28668.1 | M | N | L |
| NM_000492.1 | M | H | F |
| NM_000492.2 | M | N | L |
| NM_000492.3 | V | H | F |
| pCF1-CFTR | M | H | L |
| PGM169 | M | H | F |

CFTR amino acid 470 is polymorphic with approximately 58% of sequenced chromosomes encoding an M while approximately 42% of sequenced chromosomes encode a V. The CFTR coding sequence in both pCF1-CFTR and pGM169 encode the more frequent M allele sequence.

CFTR amino acid 620 was erroneously reported as an N in the original sequence and in NCBI Accession No: NM_{—}000492.2. The correct amino acid is H. The CFTR coding sequence in both pCF1-CFTR and pGM169 is H.

CFTR amino acid 833 is polymorphic with F being the most prevalent amino acid, though L is found rarely. The original CFTR cDNA sequence and the widely used DNA fragments deposited at the American Type Culture Collection contained an example of the rare L allele. Consequently, many widely used CFTR cDNA sequences contain the rare L allele sequence. GTAC clinical trials 002, 007, 008, 009, 015 were all conducted using CFTR cDNA sequences that contained the rare L allele sequence. The CFTR coding sequence in pGM169 encodes the more frequently found F amino acid.

The CFTR coding sequence in pGM169, nucleotides 738-5310, is provided by a CFTR cDNA termed soCFTR2. Importantly, the protein sequence encoded by soCFTR2 is identical to the full length wild type CFTR protein described by NCBI Accession No: NM_{—}000492.1. However, the DNA sequence of soCFTR2 differs considerably from the DNA sequence described by NM_{—}000492.1 as the codon usage was constrained to eliminate CpG motifs and to increase the efficiency of mRNA translation.

### Non-Coding Sequences

Transcription from the hCEFI enhancer/promoter is terminated within the CpG-free bovine growth hormone poly-adenylation sequence found at pGM169 nucleotides 5186-5387. This region is highly homologous to nucleotides 75-276 (NCBI Accession No: AF117350.1) in the Bos taurus growth hormone gene except that all CG dinucleotides have been substituted for TG dinucleotides. Post-transcriptional cleavage of the CFTR containing mRNA and polyA tail addition is predicted to occur 15-30 nucleotides 3′ to the conserved AAUAAA poly-adenylation site found at pGM169 nucleotides 5276-5281.

### Bacterial Origin of Replication and Selection

Purification of pGM169 is facilitated by replication of the plasmid DNA in its bacterial host. A CpG-free R6K bacterial origin of replication is found at pGM169 nucleotides 5389-5660. This region is highly homologous to nucleotides 327-96 (NCBI Accession No: AY608912.1) of plasmid pFL129 except that all CG dinucleotides have been substituted for TG dinucleotides. To facilitate pGM169 selection, a CpG-free kanamycin resistance gene is found at pGM169 nucleotides 6483-5668. Translation of the kanamycin resistance gene product is facilitated by a Shine-Dalgarno ribosome binding site at nucleotides 6495-6490 and a synthetic CpG-free bacterial promoter at nucleotides 6549-6490. These sequences responsible for bacterial replication and selection lack significant homology to any human sequences in the NCBI database at the time of drafting this patent application.

Verification of pGM169 Construction

The completed pGM169 was digested with a variety of restriction enzymes and the resulting DNA fragments were analysed by gel electrophoresis. The resulting fragment sizes matched the sizes calculated from the predicted sequence. The seed stock of pGM169 used to create the GMP master cell bank was completely sequenced with ≧four-fold base redundancy and was found to completely match the predicted sequence. pGM169 isolated from the master cell bank was completely sequenced under GLP conditions and was also found to completely match the predicted sequence.

The non-viral CpG dinucleotide-free plasmid has a nucleic acid sequence at least 90% identical to the nucleic acid sequence of pGM169 as set forth in SEQ ID NO:1.

This embodiment embraces plasmids that has a nucleic acid sequence at least 90%, at least 95%, at least 96%, at least 97%, at least 98% and at least 99% identical to the nucleic acid sequence of pGM169 as set forth in SEQ ID NO:1.

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994).

Preferably non-viral CpG dinucleotide-free plasmid comprises the nucleic acid sequence of SEQ ID NO:1; more preferably the non-viral vector consists of the nucleic acid sequence of SEQ ID NO:1.

Methods of preparing plasmid DNA are well known in the art. Typically, they are capable of autonomous replication in an appropriate host cell.

Host cells containing (e.g. transformed, transfected, or electroporated with) the plasmid may be prokaryotic or eukaryotic in nature, either stably or transiently transformed, transfected, or electroporated with the plasmid. Suitable host cells include bacterial, yeast, fungal, invertebrate, and mammalian cells. Preferably the host cell is bacterial; more preferably E. coli.

Host cells can then be used in methods for the large scale production of the plasmid. The cells are grown in a suitable culture medium under favourable conditions, and the desired plasmid isolated from the cells, or from the medium in which the cells are grown, by any purification technique well known to those skilled in the art; e.g. see Sambrook et al, supra.

The pharmaceutical composition the first aspect of the invention is a complex of the non-viral CpG dinucleotide-free plasmid with GL67A lipid mixture.

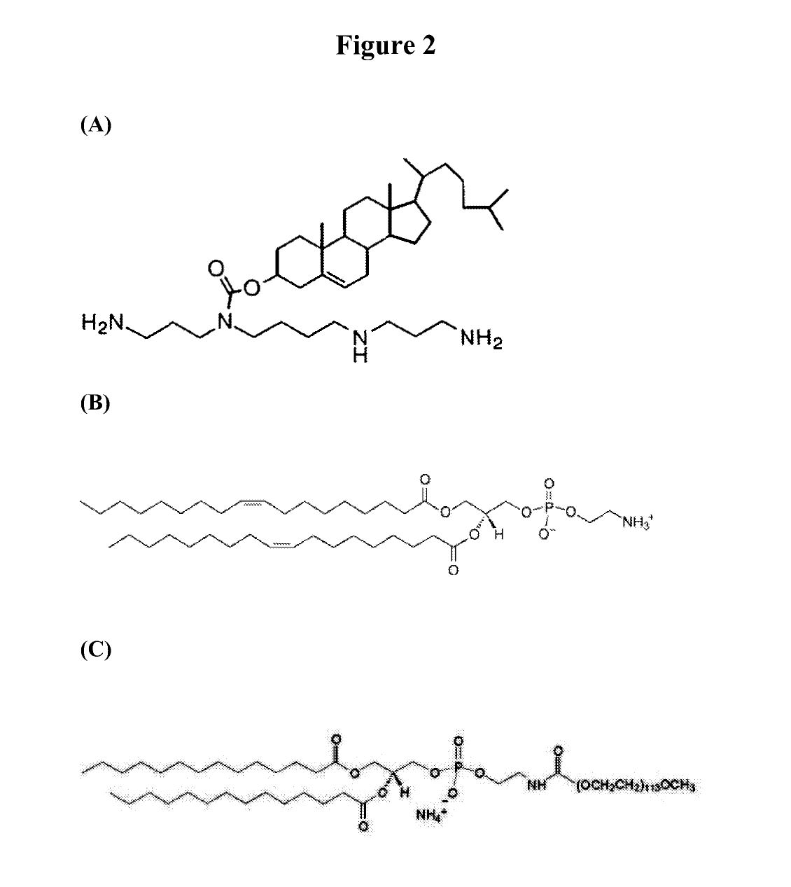

The cationic lipid mixture GL67A is a mixture of three components—GL67, DOPE and DMPE-PEG₅₀₀₀. The structure of the components is shown in FIG. 2. They are formulated at a 1:2:0.05 molar ratio.

The cationic lipid GL67, Cholest-5-en-3-ol (3β)-,3-[(3-aminopropyl) [4-[(3-aminopropyl)amino]butyl]carbamate], (CAS Number: 179075-30-0), consists of an amine (spermine) and a lipid component (cholesterol) linked together via a carbamate linkage. Genzyme Inc. (Haverhill, UK) manufactures GL67 to GMP.

DOPE, 1,2-dioleoyl-sn-glycero-3-phosphoethanolamine, is a naturally occurring neutral lipid. GMP DOPE is provided by Avanti Polar Lipids (Alabaster, Ala., USA) and is accepted based on the manufacturers' certificate of analysis. Each lot of DOPE is analysed to confirm identity by TLC, HPLC, UV-spectroscopy, gas chromatography and physical examination. The purity of the material is confirmed by HPLC (>99%).

DMPE-PEG₅₀₀₀, 1,2-Dimyristoyl-sn-Glycero-3-Phosphoethanolamine-N-[methoxy(Polyethylene glycol)5000], is 1,2-dimyristoyl-sn-glycero-3-phosphoethanolamine linked to polyethylene glycol monomethylether (average molecular weight ca. 5000) via a carbamate linkage. GMP DMPE-PEG5000 is provided by Avanti Polar Lipids (Alabaster, Ala., USA) and is accepted based on the manufacturers' certificate of analysis. Each lot of DMPE-PEG5000 is analysed to confirm identity by TLC, HPLC, and physical examination. The purity of the material is confirmed by HPLC (>95%).

A protocol for the preparation of the cationic lipid mixture GL67A is provided in the accompanying examples. Briefly, individual constituents are first dissolved in t-butanol:water (90:10%) and then mixed in appropriate quantities to obtain a GL67 to DOPE to DMPE-PEG5000 molar ratio of 1:2:0.05. After sterile filtration the lipid mixture is dispensed into individual 10 mL glass lyophilisation vials such that each vial contains 39±10 mg GL67, 94±23 mg DOPE, and 18±5 mg DMPE-PEG. The total amount of lipid in a vial is 128 to 172 mg. The vials are freeze-dried under nitrogen gas for approximately 94 hours at temperatures ranging from −50° C. to +10° C. The vials are capped with aluminium crimp caps, coded and stored at −80° C.

Total amount of lipid/ml when reconstituted in water: 24.4-32.8 mg/ml. Total amount of lipid/ml when mixed with plasmid DNA: 12.2-16.4 mg/ml. Total amount of lipid/5 ml of composition: 61-82 mg dose.

The pharmaceutical composition of the first aspect of the invention is a complex of the non-viral CpG dinucleotide-free plasmid with GL67A lipid mixture.

A method for the preparation of the non-viral CpG dinucleotide-free plasmid/GL67A lipid mixture complex is provided below in relation to the third aspect of the invention. It is preferred that the pharmaceutical composition of the first aspect of the invention comprises a complex prepared according to the method of the third aspect of the invention.

The pharmaceutical composition of the first aspect of the invention can be used as a medicament in a method of treating cystic fibrosis.

Means of identifying a patient who may be suffering from cystic fibrosis are known in the art.

In 1998, the Cystic Fibrosis Foundation issued a consensus statement regarding the diagnosis of CF. According to the panel, the diagnosis of CF should be made on the basis of one or more characteristic phenotypic features: history of a CF sibling, presence of a positive newborn screening test, and laboratory confirmation of a CFTR abnormality by an abnormal sweat chloride test, identification of mutations in a gene known to cause CF, or in vivo demonstration of an ion transport abnormality across the nasal epithelium. However, if these classic criteria as described by the committee are not present, CF still cannot be ruled out in its entirety. In patients who present later in childhood or in early adulthood, these classic criteria might not be present. In these patients, typical pulmonary symptoms or GI symptoms may be absent, and instead pancreatitis, male infertility, or sinusitis or nasal polyps may be present. Further information concerning how to conduct these tests is readily available in the art, and from appropriate website, e.g. http://www.cftrust.org.uk/.

Accordingly, from the information provided above and from further information known in the art, the skilled person can readily identify a patient having cystic fibrosis and hence may benefit from treatment with the pharmaceutical composition of the first aspect of the invention

The pharmaceutical composition of the first aspect of the invention can be used as a medicament in a method of treating cystic fibrosis.

As discussed above and in further detail in the accompanying examples, the present inventors have determined that unit doses of 10 mls and 20 mls of the pharmaceutical composition of the first aspect of the invention, containing 2.65 mg/ml non-viral CpG dinucleotide-free plasmid and GL67A lipid mixture at a concentration of 14.31 mg/ml, caused unacceptable side-effects in the subjects. However, surprisingly doses of less than 10 mls of the pharmaceutical composition were tolerated with no apparent side-effects. Moreover, it can be seen that the results of the trials demonstrated that this unit dose of pharmaceutical composition showed statistically significant evidence of a positive treatment effect.

The quantity of non-viral CpG dinucleotide-free plasmid and GL67A lipid mixture to be used may vary from treatment to treatment, depending on patient to be treated. It will be appreciated that quantity to be used depends on a number of different factors, including the age of the patient, the severity of the cystic fibrosis, contraindications, if any, and the judgement of the attending physician. Variations in quantity can be adjusted using standard empirical routines for optimisation.

An appropriate effective amount will fall in a relatively broad range but can be readily determined by one of skill in the art by routine trials. The “Physicians Desk Reference” and “Goodman and Gilman's The Pharmacological Basis of Therapeutics” are useful for the purpose of determining the amount needed.

Hence the pharmaceutical composition of the first aspect of the invention may comprise 9 mls, 8 mls, 7 mls, 6 mls, 5 mls, 4 mls, 3 mls, 2 mls, 1 ml or less of the complex containing 2 mg/ml to 3 mg/ml, and (ii) GL67A lipid mixture at a concentration of 10 mg/ml to 20 mg/ml, as well as quantities falling between all the specific amounts listed above; e.g. 9.5 ml, 8.5 mls, as can be appreciated.

As can be seen in the accompanying examples, the inventors have determined that 5 mls of pharmaceutical composition was both well tolerated and showed statistically significant evidence of a positive treatment effect.

Hence a preferred embodiment of the invention is wherein composition comprises 5 mls of the non-viral CpG dinucleotide-free plasmid/GL67A lipid mixture complex.

Though cystic fibrosis can affect a number of different tissues and organs in the human body, the lungs are one of the most affected and lung disorders are a leading cause of fatality for cystic fibrosis sufferers. In an attempt to alleviate the effects of cystic fibrosis the present inventors investigated the effectiveness of administering the medicament of the first aspect of the invention to the lungs.

In recent years a number of attempts have been made to treat lung disorders with aerosolised gene therapy agents. There are a number of advantages to this form of therapy. First, aerosolized gene therapy provides a direct, noninvasive means for targeted delivery to different regions of the lung. Second, this route of administration delivers a high dose to the target site. Third, aerosolized gene therapy causes fewer adverse effects than intravenous administration.

The goal of aerosolized gene therapy in treating CF is to reconstitute CFTR function and normal chloride channel function in the lungs. One challenge to be overcome is to deliver an adequate dose of medicament to the affected cells while at the same time ensuring that the medicament has not been degraded during nebulisation. This latter point is of key importance. Plasmid DNA is extremely shear-sensitive and hence prone to rapid degradation during nebulisation, though some protection during aerosol generation is afforded when complexed with gene therapy agents, such as the GL67A lipid mixture.

Importantly, the level of protection to the plasmid DNA, and hence the efficacy of aerosolised formulations, varies significantly between gene therapy agent and between nebulisers. As a result, development of any viable aerosol gene therapy medicament is dependent upon the identification of not only a suitable gene and gene therapy agent, but also a compatible nebuliser device for the chosen formulation.

Nebulizers use oxygen, compressed air or ultrasonic power to break up medical solutions/suspensions into small aerosol droplets that can be directly inhaled from the mouthpiece of the device. The definition of an aerosol is a “mixture of gas and liquid particles,” and the best example of a natural occurring aerosol is “mist” (being formed when small vaporized water particles mixed with hot ambient air are cooled down and condense into a fine cloud of visible airborne water droplets). When using a nebulizer for inhalation therapy with medicine to be administered directly to the lungs, it is important to note that inhaled aerosol droplets can only penetrate into the narrow branches of the lower airways if they have a small diameter of 1-5 micrometers. Otherwise they are only absorbed by the mouth cavity, where the effect is low.

Against this background the present inventors investigated the aerosolisation of the pharmaceutical composition of the first aspect of the invention by a number of different nebulisers.

In particular, they focused on aerosolising the pharmaceutical composition of the invention using jet nebulisers, which are driven either by a portable compressor or from a central air supply: essentially, a high-speed airflow through a narrow nozzle orifice entrains and disperses the liquid into droplets.

From that research they have identified an effective for the treatment of cystic fibrosis, a preferred aerosolised pharmaceutical composition is characterised as having a droplet size having a Mass Median Aerodynamic Diameter (MMAD) of less than 5 μm, and having a Fine Particle Fraction (FPF defined as the proportion of aerosol contained within droplets with MMADs less than 5 μm) greater than 50%; and having greater than 50% of the total aerosolised plasmid delivered intact.

Mass Median Aerodynamic Diameter (MMAD) is a well known means of characterizing particle size in an aerosol. The measurement, with the geometric standard deviation, used to describe the particle size distribution of any aerosol statistically, based on the weight and size of the particles. Means of calculating the MMAD of an aerosol are well known in the art. An example of how to calculate the MMAD of an aerosol of the medicament of the invention is provided in the accompanying examples.

Fine Particle Fraction is a measure of the proportion of particles having the desired size characteristic. For the present invention, this is defined as the proportion of aerosol contained in droplets of less than 5 μm in diameter. Again, means of calculating the Fine Particle Fraction of an aerosol are well known in the art. An example of how to calculate the Fine Particle Fraction of an aerosol of the medicament of the invention is provided in the accompanying examples.

Preferably the pharmaceutical composition is formulated as an aerosol, wherein the aerosol has a droplet size having a Mass Median Aerodynamic Diameter (MMAD) of less than 5 μm, and having a Fine Particle Fraction (FPF defined as the proportion of aerosol contained within droplets with MMADs less than 5 μm) greater than 50%; and having greater than 50% of the total aerosolised plasmid delivered intact. More preferably the aerosol has a Mass Median Aerodynamic Diameter (MMAD) in the range 2-5 μm and a Fine Particle Fraction (FPF) greater than 60%.

As discussed above, the present inventors have determined that the pharmaceutical composition of the first aspect of the invention has much utility when used in a method of treating cystic fibrosis.

Accordingly, a second aspect of the invention provides a medicament comprising as a unit dose the pharmaceutical composition of the first aspect of the invention for use in a method of treating cystic fibrosis.

As used herein “unit dose” refers to the amount of medicament administered to the patient in a single treatment stage as part of an overall treatment regimen.

By “treatment of cystic fibrosis” we include where the medicament alleviates or suppresses the symptoms of cystic fibrosis. Hence the aspect of the invention is not to be considered as restricted to curing cystic fibrosis.

Preferably the method comprises administering the medicament as an aerosol to a patient in need thereof.

As demonstrated in the accompanying examples, they have found that breath-actuated nebulisers are preferably used when administering an aerosolised medicament to the patient. This is because the breath enhancement mechanism increases the proportion of aerosol generated during patient inhalation.

The inventors proceeded to investigate which type of breath-actuated nebuliser can be best used to administer the medicament of this aspect of the invention.

They determined that preferably the aerosol is generated from a breath-actuated nebuliser device with a formulation capacity of between 2 ml and 10 ml.

Preferably the breath-actuated nebulizer is capable of generating stable formulation aerosols for the duration of aerosol delivery. By “stable aerosol generation” we include where the aerosols have the physical characteristics described above in relation to the first aspect of the invention, i.e. a droplet size having a Mass Median Aerodynamic Diameter (MMAD) of less than 5 μm, and having a Fine Particle Fraction (FPF defined as the proportion of aerosol contained within droplets with MMADs less than 5 μm) greater than 50%; and having greater than 50% of the total aerosolised plasmid delivered intact.

In addition, over 65% of the total aerosol output from the breath-actuated nebuliser must be generated during the inspiratory cycle (equivalent to patient inhalation) as assessed under standard simulated breathing conditions (sinusoidal breathing, tidal volume 500 ml and inspiratory:expiratory ratio of 1:1).

Also, it is preferred that the aerosol is delivered to a patient at an aerosol delivery rate of between 80 μl/min and 400 μl/min, assessed under standard simulated breathing conditions (sinusoidal breathing, tidal volume 500 ml and inspiratory:expiratory ratio of 1:1).

From the analysis provided herein, the inventors determined that it was advantageous to administering the medicament by the AeroEclipse II nebulizer, which can be obtained from Trudell Medical International 725 Third Street, London, Ontario, Canada N5V 5G4. http://www.trudellmed.com/. Details of the AeroEclipse II nebulizer may be obtained from http://www.trudellmed.com/consumer-health/aeroeclipse-ii-ban.

The inventors have identified that the AeroEclipse II nebulizer has the following characteristics: (i) capable of nebulising the medicament without excessive foaming of the formulation within the unit and concomitant interference with aerosol generation; (ii) the size characteristics of the medicament (MMAD 3.4±0.1 FPF 71.4±1.5%) were appropriate for aerosol delivery to the lungs of CF patients with optimal aerosol characteristics achieved at a nebuliser operating pressure of 50 psi; (iii) AeroEclipse II was CE marked for operation at 50 psi; (iv) breath actuation mechanism within the device permitted more efficient delivery of formulation to the lungs of patients (oral bioavailability of 83.0±2.3% at a rate of 0.17±0.01 ml/min), with a significant reduction in aerosol wastage and environmental contamination; (v) nebulisation of the medicament using the AeroEclipse II is not associated with excessive modification of the physical characteristics of the formulation (68.5±0.9% of pDNA delivered intact).

In addition to the breath-actuated nebulisers discussed above, the medicament may be administered to a subject using equipment that allows more controlled and potentially more efficient deposition of gene transfer agents.

For example, systems such as Akita which allow control of inspiration flow, as well as number and volume of breath, may be coupled to nebulisers to ensure more controlled deposition of the lipid/DNA complexes in the lungs. Further information regarding the Akita equipment may be found at, for example, www.activaero.de.

As discussed above, the present inventors have investigated the administration of aerosolised medicament to the patient using breath-actuated nebulisers. When devising an administration regimen to be used, they had reason to consider that the volumes of medicament to be administered may cause discomfort to the patient since this requires them to use a breath-actuated nebuliser for a relatively long time.

With this in mind, they have devised a preferred embodiment of the second aspect of the invention in which the method of treating cystic fibrosis comprises administering the medicament in a cyclic treatment regimen in which the patient inhales the aerosol for a period of time followed by a rest period.

As can be appreciated, the length of time in which the patient inhales the aerosol and the length of time for the rest period can vary from patient to patient and treatment cycle to cycle. However, a preferred treatment regimen cycle is wherein the patient inhales the medicament for 3 minutes followed by 2 minutes without inhaling the medicament.

As set out in the accompanying examples, the inventors have shown that the medicament used in the first aspect of the invention functions as a CFTR gene therapy agent and can achieve a long-lasting and cumulative CFTR expression that can build with successive doses. The unit doses used are well tolerated in the animal models studied. The data support progression of the administration regimen from single administration to a multidose regimen designed to generate clinical benefit in CF patients.

Accordingly a further embodiment of the second aspect of the invention is wherein the method of treating cystic fibrosis comprises repeatedly administering the unit dose of the pharmaceutical composition to a patient in need thereof; preferably according to a continuous schedule having a once-monthly dosing interval.

It can be appreciated by the skilled person that there can be some degree of flexibility within this regimen; for example, the gap between each single dose may vary from 3 to 5 weeks, as required by the clinical need of the patient. Administration regimens are tailored by the clinician, or others skilled in the pharmacological arts, based upon well known pharmacological and therapeutic considerations including, but not limited to, the desired level of therapeutic effect, and the practical level of therapeutic effect obtainable.

As discussed above, the accompanying examples provide data demonstrating the tolerance and clinical benefit of the medicament when used in the second aspect of the invention. On occasion, the inventors noted that some patients exhibited a mild systemic inflammatory response as measured by a reduction in FEV₁ value and rise in white blood cells. On investigation the inventors determined that the inflammatory response was alleviated when the patient was administered an anti-pyretic agent, such as paracetamol, following administration of the medicament.

Accordingly therefore a further embodiment of the invention is wherein the medicament further comprises an anti-pyretic agent, preferably paracetamol.

While the anti-pyretic agent may be administered within the same formulation as the non-viral CpG dinucleotide-free plasmid/GL67A lipid mixture complex, it is preferred that the agent is administered as a separate but concomitant component of the medicament.

The aerosolised medicament may also contain one or more excipients commonly used in aerosol medicaments.

For example, excipients which may be included in the medicament for use in the first aspect of the invention include but are not limited to dispersing agents, preservatives, flavorings, taste masking agents, buffers, antioxidants, anti-aggregating agents, and co-solvents. The particular excipient(s) used and the concentration of the excipient(s) are selected according to the particular medicament(s) used and the desired physical properties of the formulation. Surfactants are commonly added to suspension aerosol formulations, for example to lubricate the valve components in the inhaler device and/or improve the physical stability of the suspension. Suitable surfactants include both non-fluorinated surfactants and fluorinated surfactants known in the art.

A third aspect of the invention provides a method for preparing a medicament for the treatment of cystic fibrosis, the method comprising (i) placing a quantity of a liquid composition of a non-viral CpG dinucleotide-free plasmid comprising nucleic acid encoding a CFTR polypeptide operatively linked to hCEF1 promoter in a first compartment; (ii) placing a quantity of a liquid composition of GL67A lipid mixture (ii) in a second compartment; (iii) co-extrusion of the contents of the first and second compartments through a static mixer at a fluidic flow rate having a laminar flow defined by a Reynold number of less than or equal to 4000.

Preferably the non-viral CpG dinucleotide-free plasmid is at a concentration of 1.5 mg/ml to 3.5 mg/ml and the GL67A lipid mixture is at a concentration of 10 mg/ml to 20 mg/ml.

Preferably the compositions are mixed in a static mixer containing between 4 and 16 elements and having a diameter of between 1 mm and 5 mm. More preferably the static mixer contains 8 elements and has a diameter of 3 mm.

The present inventors have identified a gene therapy medicament which functions as a CFTR gene therapy agent, a unit dose for that medicament, and suitable administration routes and regimens. That medicament has been shown to be tolerated by subjects and importantly a unit dose of medicament showed statistically significant evidence of a positive treatment effect.

As can be appreciated, it is important to standardize the preparation of the gene therapy medicament when it is used for the treatment of the first aspect of the invention.

Non-viral gene therapy formulations, such as the medicament described herein, are typically prepared in the laboratory in relatively small volumes (<1 mL) by simply adding pDNA to a solution of lipid or polymer with subsequent mixing by repeated inversion or pipetting. Under such conditions, mixing parameters are virtually impossible to standardize leading to variability in the physical characteristics of complexes prepared in different laboratories, even when using identical starting material. Such variability is further exacerbated by attempts to scale-up mixing volumes for pre-clinical and clinical studies where differences in physical characteristics may crucially impact key in vivo outcomes including gene expression, toxicity, and biodistribution.

Against this background, the present inventors have devised a method for the preparation of a medicament to be used in the first aspect of the invention. That method is characterized in that the two components are simultaneously expelled from their respective compartments at a known flow rate.

The fluidic flow rate is defined as having a laminar flow defined by a Reynold number of less than or equal to 2300, based on the circular pipe model of flow characteristics. It has been found that such a flow rate provides a non-viral CpG dinucleotide-free plasmid/GL67A complex having physical characteristics desirable for gene therapy medicaments (as measured by hydrodynamic diameter and zeta potential) while no evidence was found for shear-related degradation of the plasmid DNA.

“Reynolds number” is a well known measure of laminar flow. Methods of determining the Reynolds number of a flow are well known in the art and can be readily used by the skilled person when performing the method of the third aspect of the invention. Laminar flow occurs when ReD<2000 and turbulent flow occurs when ReD>4000. In the interval between 2300 and 4000, laminar and turbulent flows are possible (‘transition’ flows), depending on other factors. Preferably the Reynolds number is less than or equal to 2300, in which case the fluidic flow will be a laminar flow.

The aspects of the invention provided above relate to liquid pharmaceutical compositions, medicaments comprising said compositions, and a method of preparing the liquid pharmaceutical compositions.

However it can be readily appreciated by the skilled person that lyophilized formulations of the same pharmaceutical compositions and associated medicament can also be prepared and is also envisaged by the present inventors.

Accordingly, a fourth aspect of the invention include a lyophilized pharmaceutical composition comprising a complex of (i) a non-viral CpG dinucleotide-free plasmid comprising nucleic acid encoding a CFTR polypeptide operatively linked to hCEF1 promoter and (ii) GL67A lipid mixture.

Methods of preparing lyophilized formulations are well known in the art. For example U.S. Pat. No. 5,811,496 (hereby incorporated by reference) provides a detailed protocol for the preparation of a lyophilized formulation which includes polynucleotide.

Generally, liquid pharmaceutical compositions of the invention are prepared (optionally according to the method of the third aspect of the invention) and subsequently lyophilized using, for example, a programmable tray dryer at a product eutectic temperature of −30° C.

The lyophilized pharmaceutical compositions of the invention can be stored then rehydrated before administration to a patient. When reconstituted for use, the quantity of liquid to be used is such that the pharmaceutical composition is according to the first aspect of the invention, i.e. the wherein the non-viral CpG dinucleotide-free plasmid is at a concentration of 2 mg/ml to 3 mg/ml, and the GL67A lipid mixture is at a concentration of 10 mg/ml to 20 mg/ml.

Hence preferably the composition contains between 2 mg to 30 mg of the non-viral CpG dinucleotide-free plasmid and 10 mg to 200 mg of GL67A lipid mixture. More preferably pharmaceutical composition comprises between 2.65 mg to 26.5 mg of the non-viral CpG dinucleotide-free plasmid and 14.31 mg to 143.1 mg of the GL67A lipid mixture.

A preferred embodiment of the invention is wherein a cryoprotectant is included in the pharmaceutical composition. The cryoprotectant stabilizes the polynucleotide plasmid component of the composition allowing the lyophilized pharmaceutical composition to be stored for extended periods of time and then rehydrated prior to use.

Cryoprotectant compounds comprise carbohydrates, preferably lactose and sucrose, but also glucose, maltodextrins, mannitol, sorbitol, trehalose, and others. Betaines prolines, and other amino acids may also be useful. Preferably, the invention comprises plasmid complexes cryoprotected with lactose at concentrations of about 1.25% to about 10% (w/vol).

In a further embodiment, the lyophilized formulations may be milled or sieved into a dry powder formulation which may be used to deliver the polynucleotide complex. Once the powder contacts the desired tissue, it rehydrates, allowing delivery of the polynucleotide complex. In a preferred embodiment, a dry powder formulation is used to induce genetic modification of a patient's lung tissue

Further details of a suitable experimental protocol for use in this aspect of the invention can be found in U.S. Pat. No. 5,811,496.

The lyophilized formulations of the fifth aspect of the invention can also be used in the medicament according to the second aspect of the invention for use in the treatment of cystic fibrosis.

A fifth aspect of the invention provides a kit of parts comprising: (i) a pharmaceutical composition according to the first aspect of the invention; (ii) a breath-actuated nebulizer as discussed in relation to the second aspect of the invention; and optionally (ii) a protocol for the administration of the medicament to a patient using the breath-actuated nebulizer.

Preferably the breath-actuated nebulizer is an AeroEclipse II nebulizer.

The present inventors have also investigated methods for monitoring the success of treatments of cystic fibrosis. They sought to determine an optimal set of clinical assays that could be used to gauge the effectiveness of treatment. Accordingly, they conducted a longitudinal study of patients with cystic fibrosis who were to be treated with intravenous antibiotics. A series of more than 15 biomarkers were measured for each patient before, during and after treatment. From the data prepared, they identified a set of biomarkers that could distinguish CF from non-CF patients, and have low enough variability that they can be used to monitoring the success of treatments of cystic fibrosis.

Hence a sixth aspect of the invention provides a method of monitoring the effectiveness of a treatment for cystic fibrosis, comprising (i) assessing prior to treatment one or more biomarkers selected from a group comprising FEV₁, Lung Clearance Index, Computed Tomography scan and Quality of Life questionnaire; and (ii) assessing the same biomarker(s) in the patient during and/or after treatment.

Preferably the method comprises monitoring two or more, three or more, or all four of the FEV₁, Lung Clearance Index, Computed Tomography scan and Quality of Life questionnaire biomarkers. Any combination of biomarkers can be used. However, preferably at least one of the biomakers is FEV₁.

Preferably the treatment for cystic fibrosis is the medicament of the second aspect of the invention.

Until the present invention it was not known or appreciated that this combination of biomarkers could be used to distinguish CF from non-CF patients, and have low enough variability that they can be used to monitoring the success of treatments of cystic fibrosis.

Methods of measuring each of the biomarkers are known. As way of example, specific examples of methods of measuring each of the biomarkers are provided herein and in the accompanying example section.

In particular, FEV₁ can be measured by spirometry using for example a pocket electronic spirometer (e.g. Piko-1, Ferraris Respiratory, Hertford, UK). FEV₁ provides an absolute value that can be used to assess the effectiveness of the treatment.

The present inventors have investigated how best to measure the spirometry value of a patient. As presented in Example 11 below, they compared several methods of calculating spirometry values, and noted that it is preferable to use reference equations which bridge the transition from child to adulthood. In particular, they noted that patients transitioned between paediatric and adult reference ranges show a greater slope of change in their FEV₁ score with Rosenthal/Quanjer reference equations, than the Stanojevic reference equation. Accordingly, it is preferred that the FEV₁ biomarker is measured using the Stanojevic reference equation when used in the methods of the present invention. The Stanojevic reference equation is well known in the art.

Lung Clearance Index is a measure of lung physiology. The lung clearance test involves following the washout of an inert tracer gas from the lungs. It measures how efficiently gases are mixing in the lungs which in turn can be used to monitor changes in lung structure. LCI can be measured in method in which multiple breath washout is performed using a modified Innocor gas analyser and 0.2% sulphur hexafluoride (SF6) as the tracer gas. LCI is quoted as the mean of at least two reproducible repeats from washouts of satisfactory quality. LCI provides an absolute value that can be used to assess the effectiveness of the treatment.

For Computed Tomography (CT) scan CT images were scored for eight independent features using a novel scoring system. The scoring system provides independent scores for each of the eight parameters and no global score is calculated. Bronchiectasis, bronchial wall thickness, small and large airway mucus plugging were assessed for extent and severity using a semi-quantitative graded scoring system. Air trapping, consolidation and ground glass opacification were scored as a percentage of lung affected (to the nearest 5%), in each of the six. The final score represents the sum of the individual lobe scores for that feature from both radiologists, i.e. 12× the maximum score (2 radiologists×6 lobes) possible for an individual lobe. That score is then expressed in a scale of 0 to 6, in which 0 indicates normal lungs, while 6 indicates the most severely damaged lung structure. Hence the CT biomarker provides a semi-quantiative measure of lung structure.

The Quality of Life Questionnaire asks the patients, before and at intervals after a treatment, questions such as the amount of sputum they produce, their level of breathlessness, as well as more general questions about how they are feeling. It is a self-graded symptom score. Patients scored each of seven symptom related questions on a five point scale from −2 (much worse than normal) to +2 (much better than normal), with zero representing no change from normal for them. The final symptom score, obtained by summing the individual question scores, thus ranges from −14 to +14. Likewise, a negative change in symptom score after treatment represents deterioration in symptoms, a positive score represents improvement and a zero score would be consistent with no overall change in patient-reported symptoms. Hence the Quality of Life Questionnaire provides a semi-quantiative measure of the effectiveness of the treatment.

In use, the biomarkers listed above will be assessed prior to treatment, then assessing the same set of biomakers in a patient during and/or after treatment. The scores provided by each biomarker will be assessed and this will give a measure of the effectiveness of the treatment. For example, the treatment may elevate FEV₁ from 58% to 65%, or a CT biomarker reduction from 4 to 2, indicates that the treatment is effective at treating cystic fibrosis.

The sixth aspect of the invention provides a method of monitoring the effectiveness of a treatment for cystic fibrosis. The same set of biomarkers has also been used to determine whether a patient is suitable for treatment with the medicament of the invention.

From this study the inventors have identified that FEV₁ is the most important biomarker when assessing whether a patient is suitable for treatment the medicament of the invention. Specifically, the inventors consider that a patient having an FEV₁ valve of 65%, preferably 70%, most preferably 76% or more, can be administered the medicament of the invention.

Hence a seventh aspect of the invention provides a method of selecting a patent for treatment with an aerosolized formulation of the medicament of the invention, comprising determining a FEV₁ value of the patient, wherein a FEV₁ value of at least 65% indicates that the patent is suitable for the treatment.

Methods of measuring the FEV₁ value of human subject are well known in the art and are discussed further above.

All of the features described herein (including any accompanying claims, abstract and drawings), and/or all of the steps of any method or process so disclosed, may be combined with any of the above aspects in any combination, except combinations where at least some of such features and/or steps are mutually exclusive.

The invention will now be further described with reference to the following Example and figures in which.

FIG. 1: Diagrammatic representation of pGM169.

FIG. 2: Structure of GL67A.

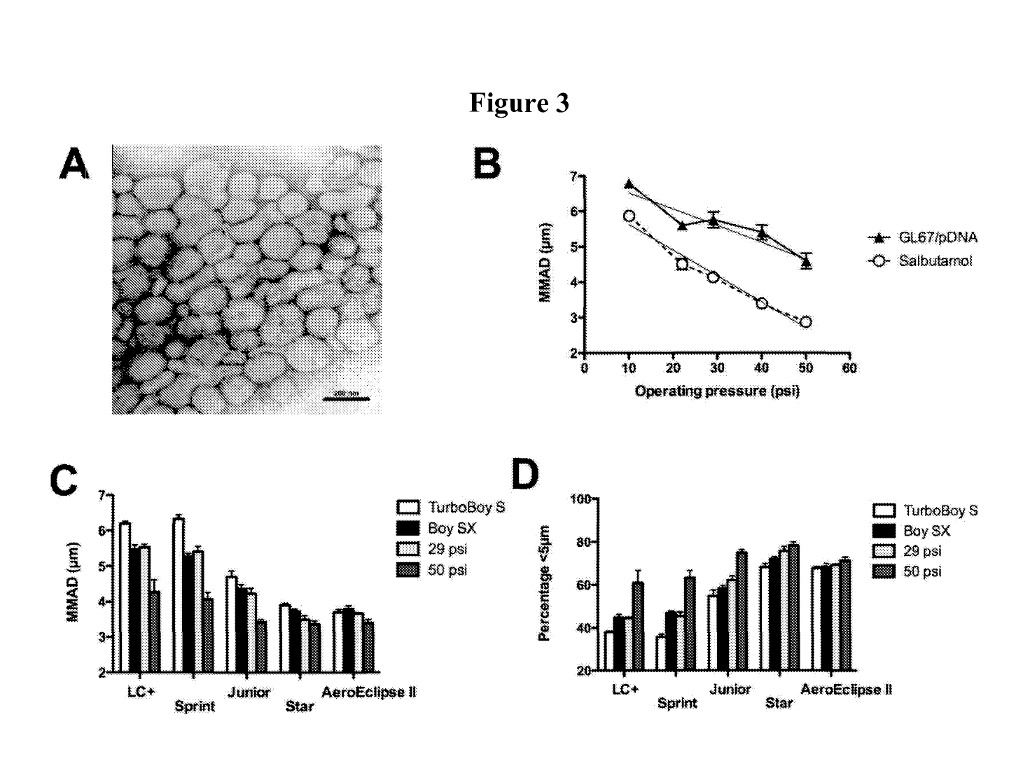

FIG. 3: Aerosol characteristics of GL67A/pDNA formulations.

(A) Electron micrograph of GL67A/pDNA aerosol formulation. (B) Comparison of aerosol droplet size between GL67A/pDNA formulations and a 1% salbutamol solution nebulised over a range of operating pressures using a Pari LC+ nebuliser. (C) Aerosol MMAD and (D) Fine Particle Fraction of GL67A/pDNA formulations nebulised using a variety of clinical jet nebulisers operated using compressed air or clinical compressor devices.

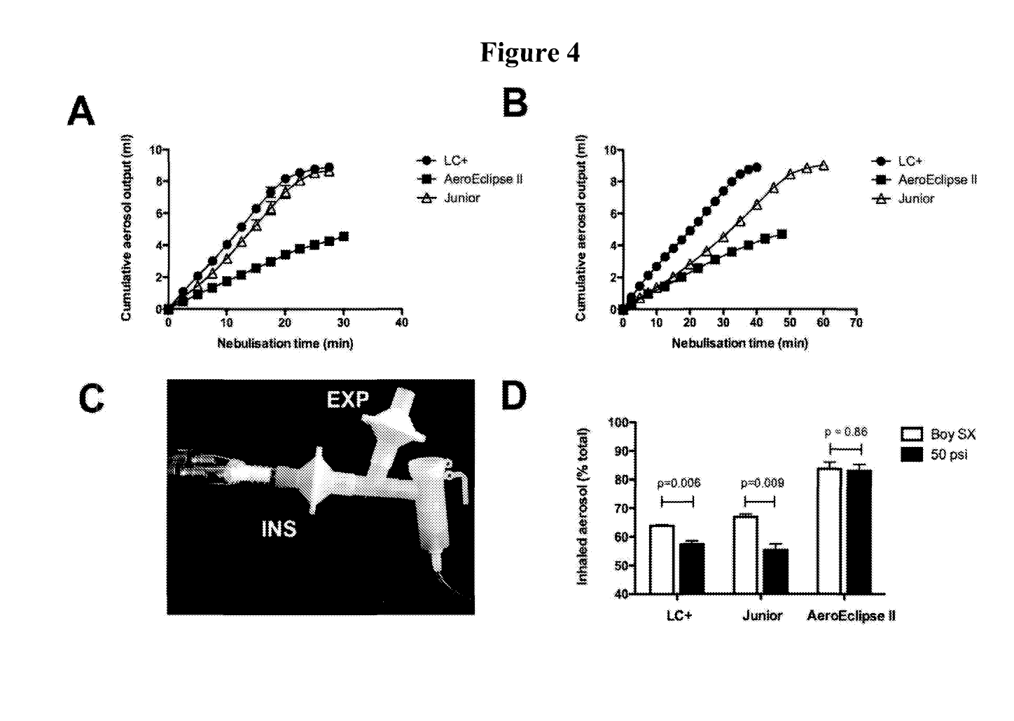

FIG. 4: Nebuliser output characteristics utilising GL67A/pDNA formulations

GL67A/pDNA aerosol output rates for the LC+, AeroEclipse II and Junior nebulisers were determined under simulated breathing conditions using (A) compressed air at 50 psi or (B) using the Pari Boy SX medical compressor. (C) Aerosol collection filters incorporated into the inspiratory (INS) or expiratory (EXP) arms of the nebuliser breathing circuit were utilised to determine GL67A/pDNA aerosol delivery efficiency under simulated breathing conditions (D).

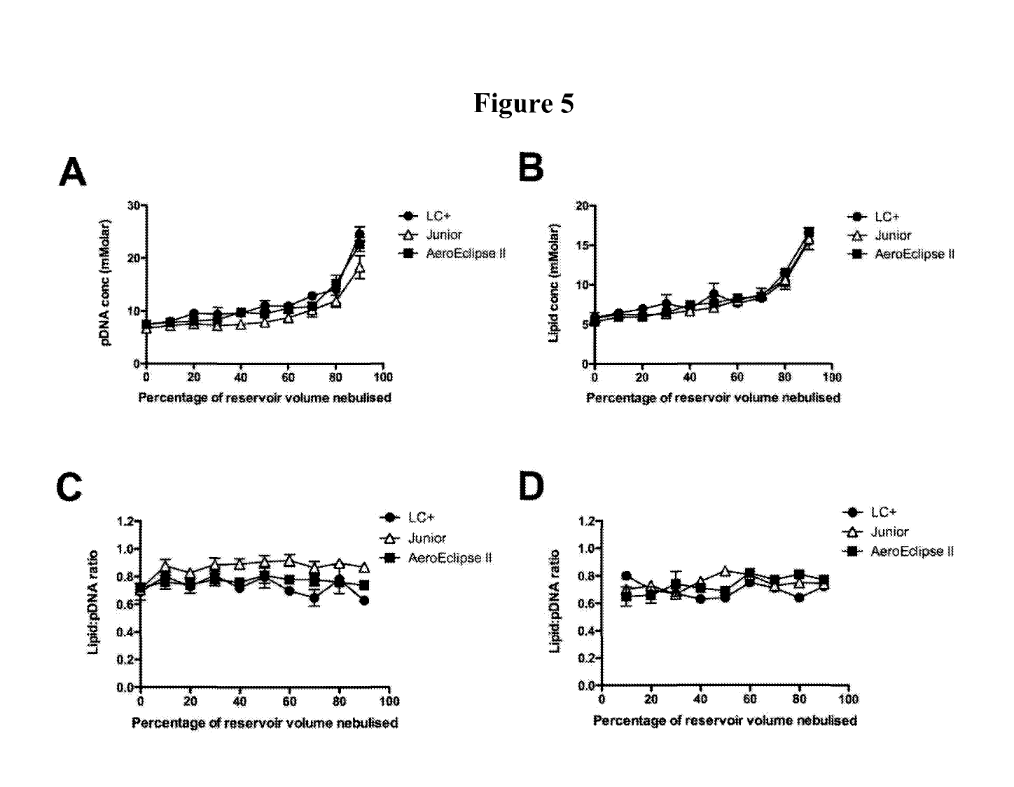

FIG. 5: Concentration of GL67A/pDNA formulations during nebulisation

The concentration of pDNA (A) and GL67A (B) were independently determined in the nebuliser reservoir at intervals during the nebulisation process using the LC+, AeroEclipse II and Junior nebulisers. The molar ratio of GL67A:pDNA was determined at intervals during nebulisation for material collected in the nebuliser reservoir (C) or collected from the generated aerosol (D).

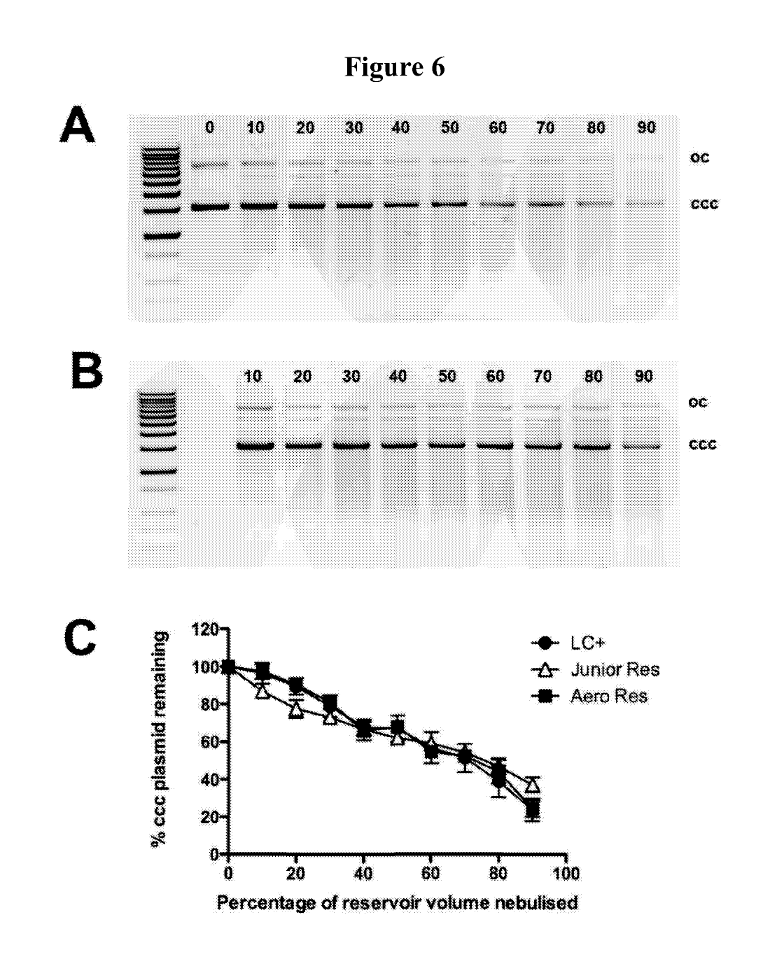

FIG. 6: Shear degradation of pDNA during nebulisation.

Electrophoretic profiles of pDNA samples removed at intervals from the AeroEclipse II reservoir (A) or collected from the generated GL67A/pDNA aerosol (B) during nebulisation. Open circular (oc) and covalently closed circular (ccc) plasmid forms are indicated. (C) Densitometric analysis of pDNA profiles from LC+, AeroEclipse II and Junior nebulisers indicating the proportion of ccc plasmid form remaining during the course of nebulisation.

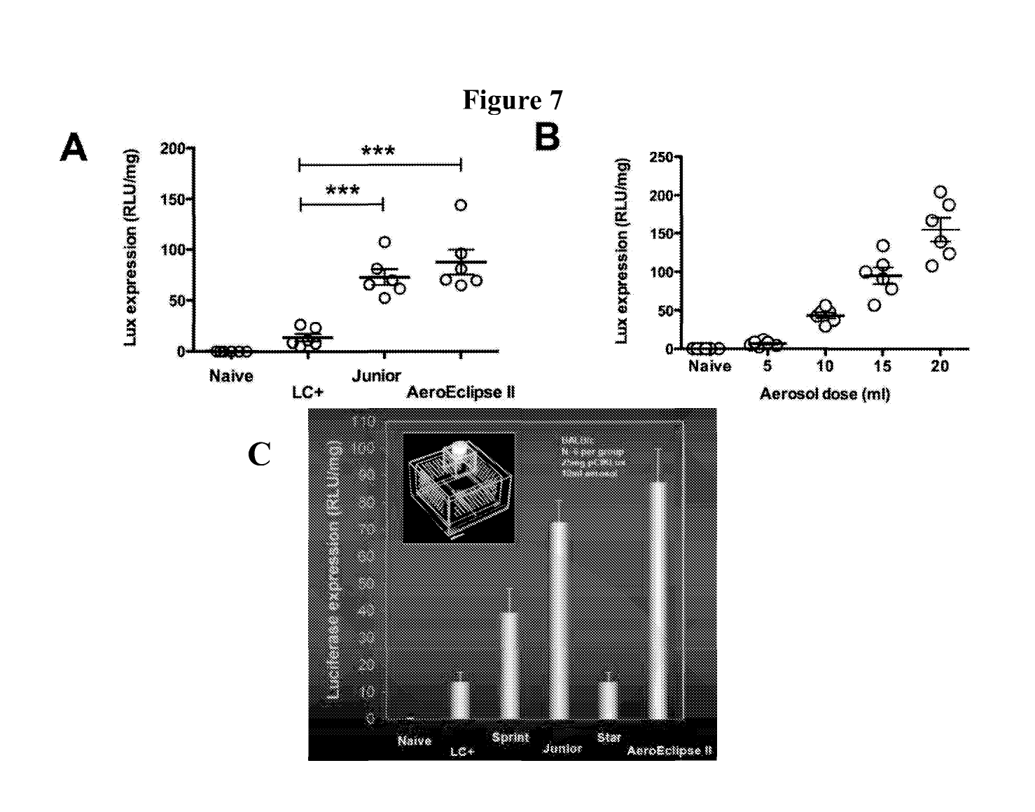

FIG. 7: Aerosol delivery of GL67A/pDNA in vivo

(A) Lung gene expression in BALB/c mice 24 hrs after aerosol delivery of 10 ml GL67A/pCIKLux complexes using the LC+, AeroEclipse II or Junior nebulisers operating at 50 psi. (B) Lung gene expression in mice exposed to increasing volumes of GL67A/pCIKLux delivered using the AeroEclipse II nebuliser operating at 50 psi; (C) Luciferase expression levels using several different nebulisers.

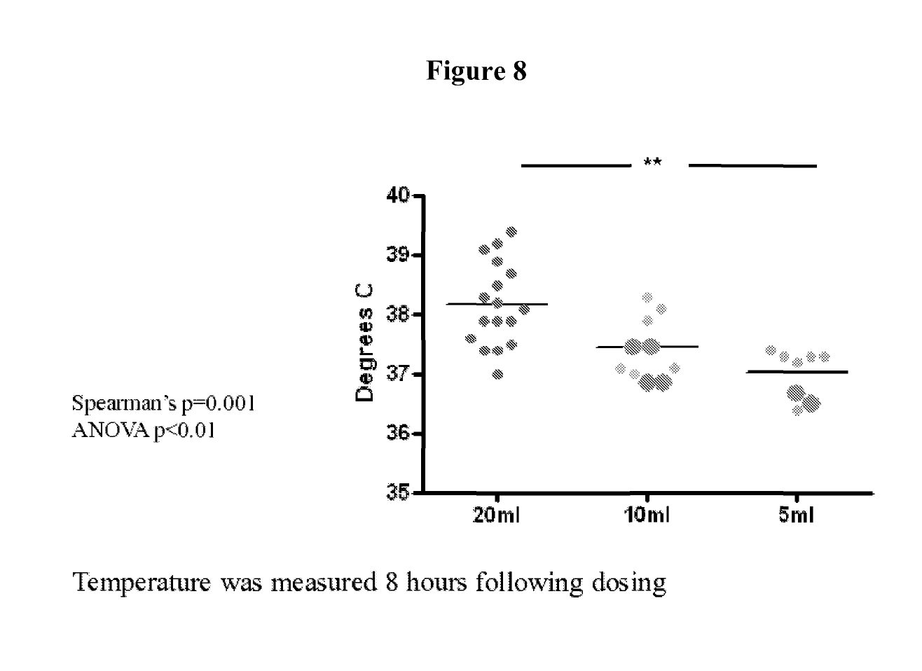

FIG. 8: Dose-related inflammatory response: temperature

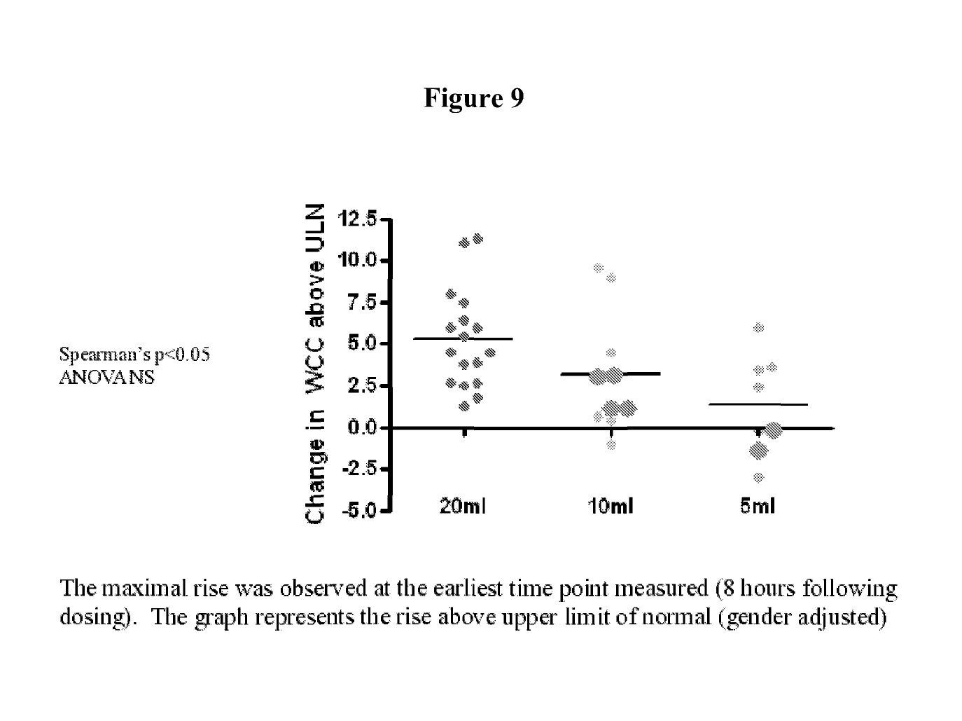

FIG. 9: Dose-related inflammatory response: peripheral white blood cell count

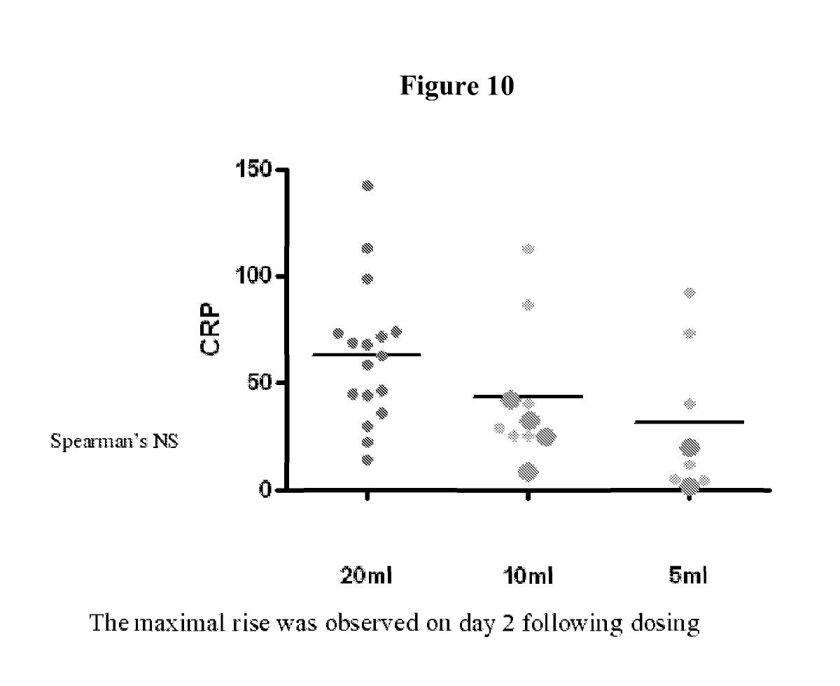

FIG. 10: Dose-related inflammatory response: C-reactive protein

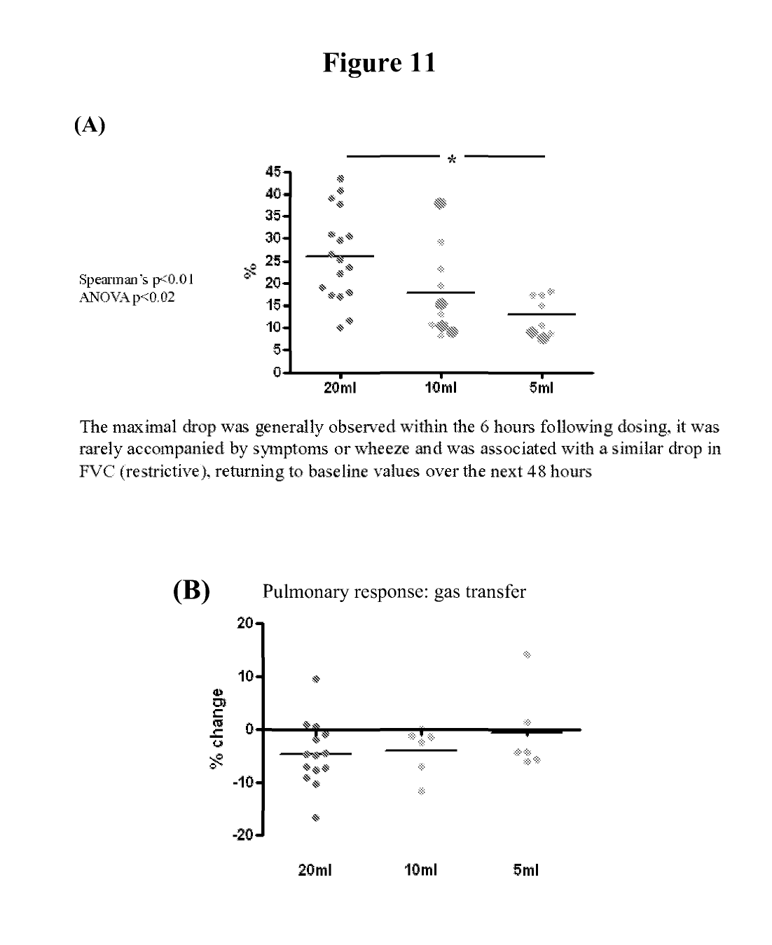

FIG. 11: Dose-related inflammatory response: (A) % age change from baseline FEV₁ and (B) change in gas transfer.

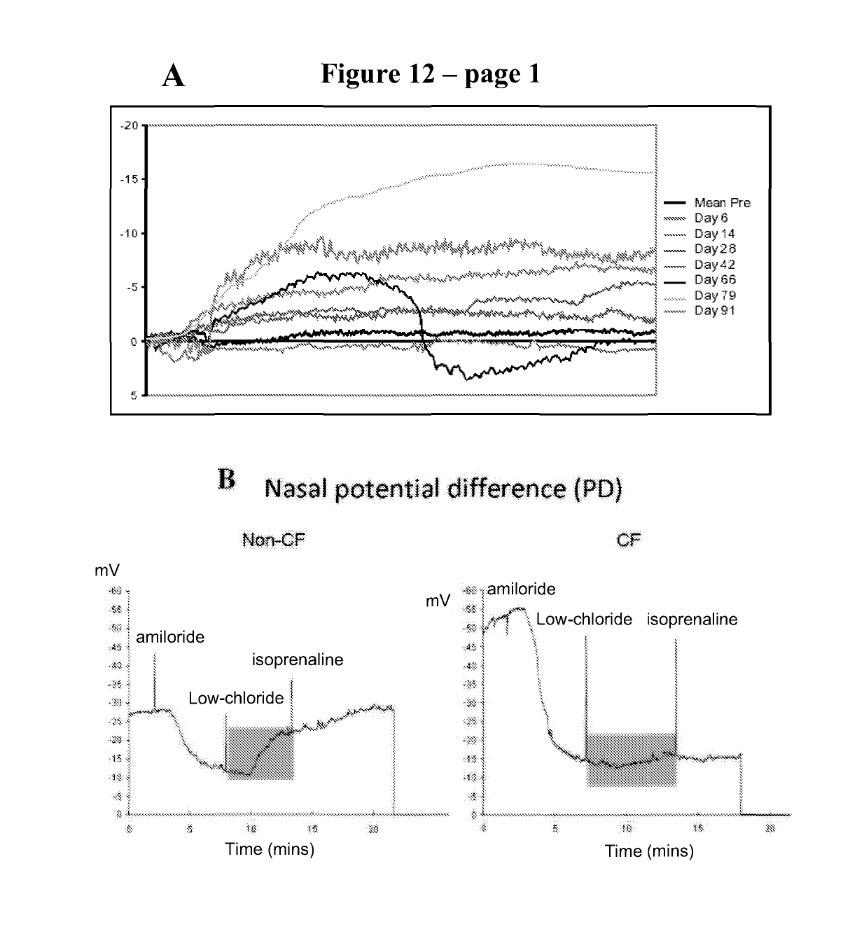

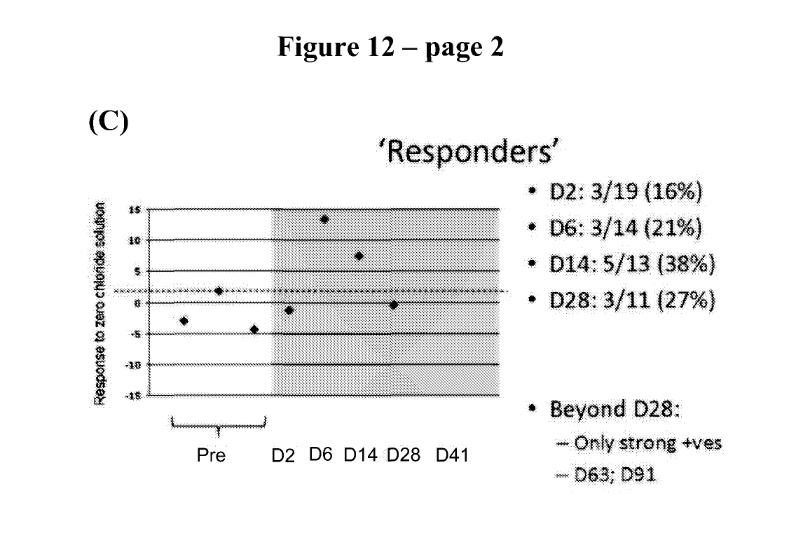

FIG. 12: A—Long term nPD correction in a treated patient; B—nasal potential difference; C—responders.

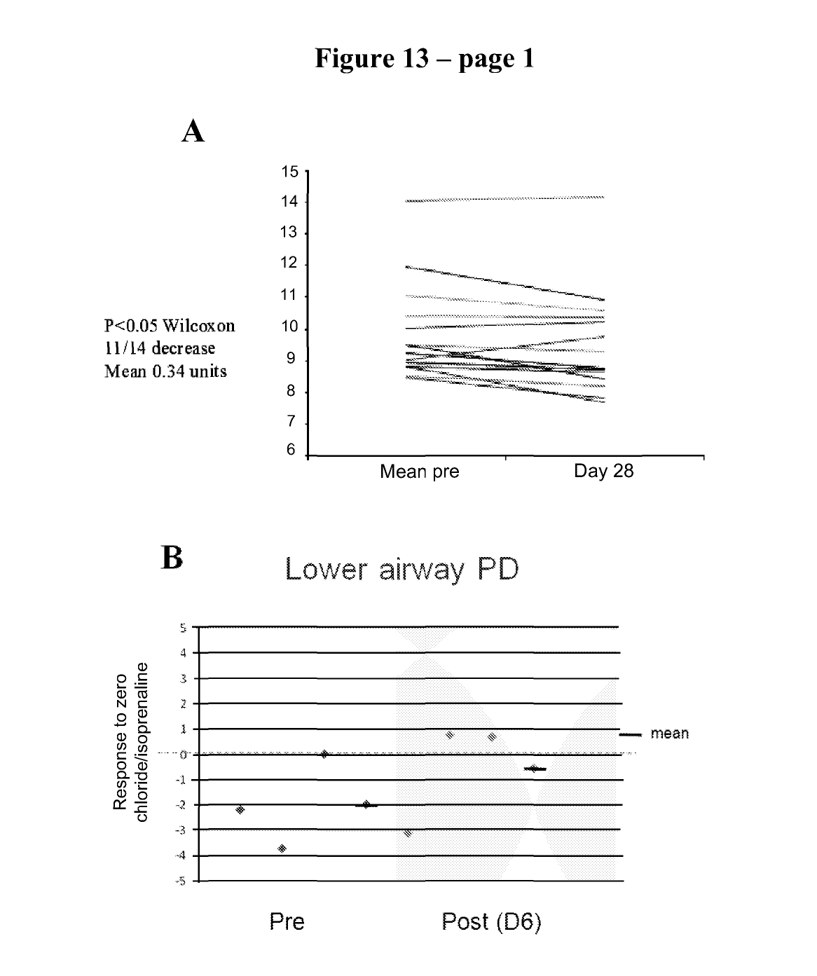

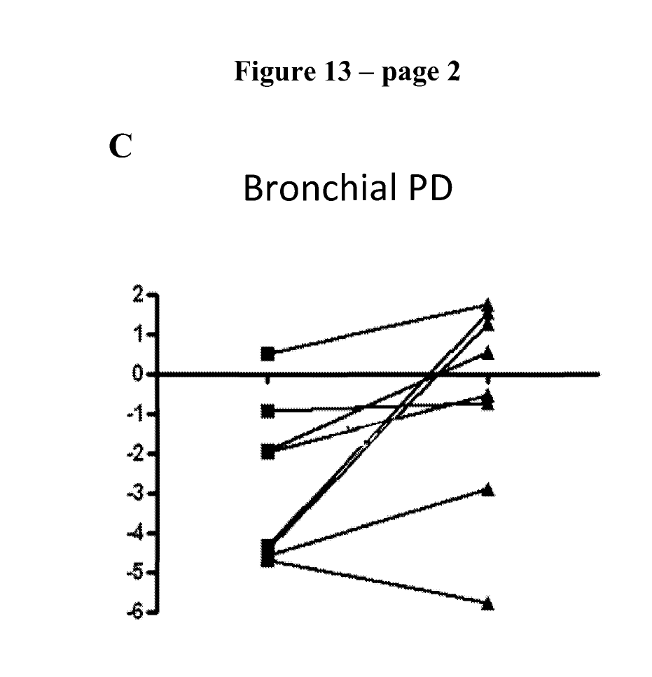

FIG. 13: A—Lung clearance index: 10 and 5 ml groups; B—Lower airway PD; C—Bronchial PD.

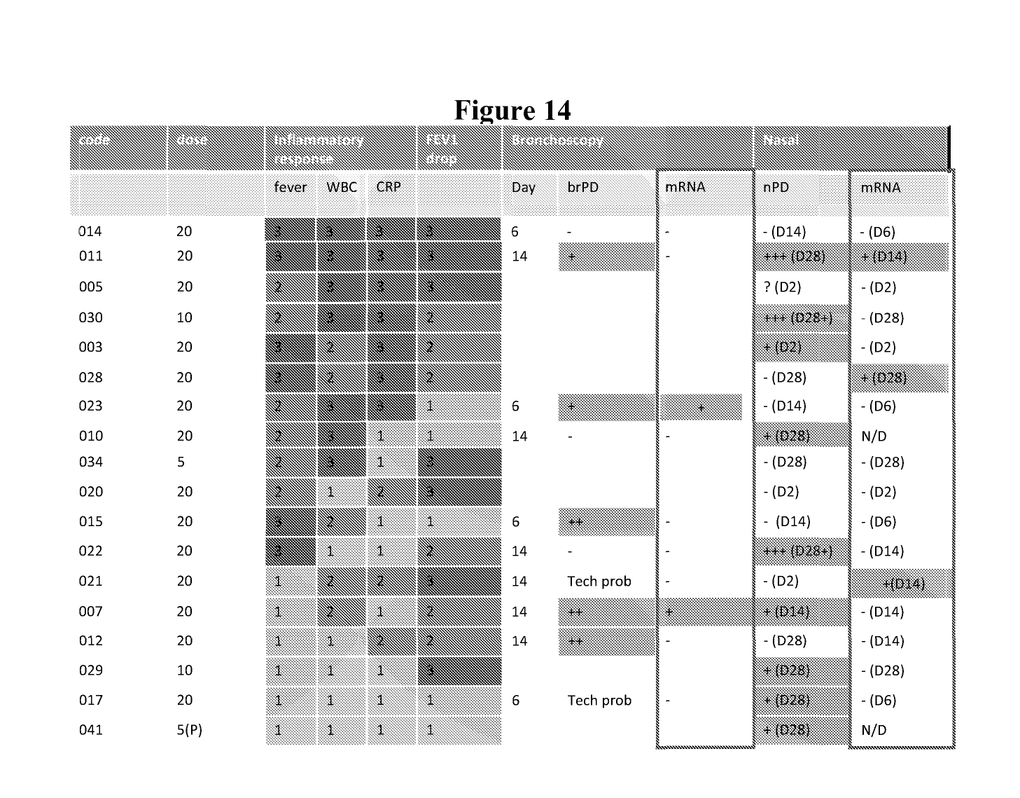

FIG. 14: Tabulation of inflammatory responses and efficacy data

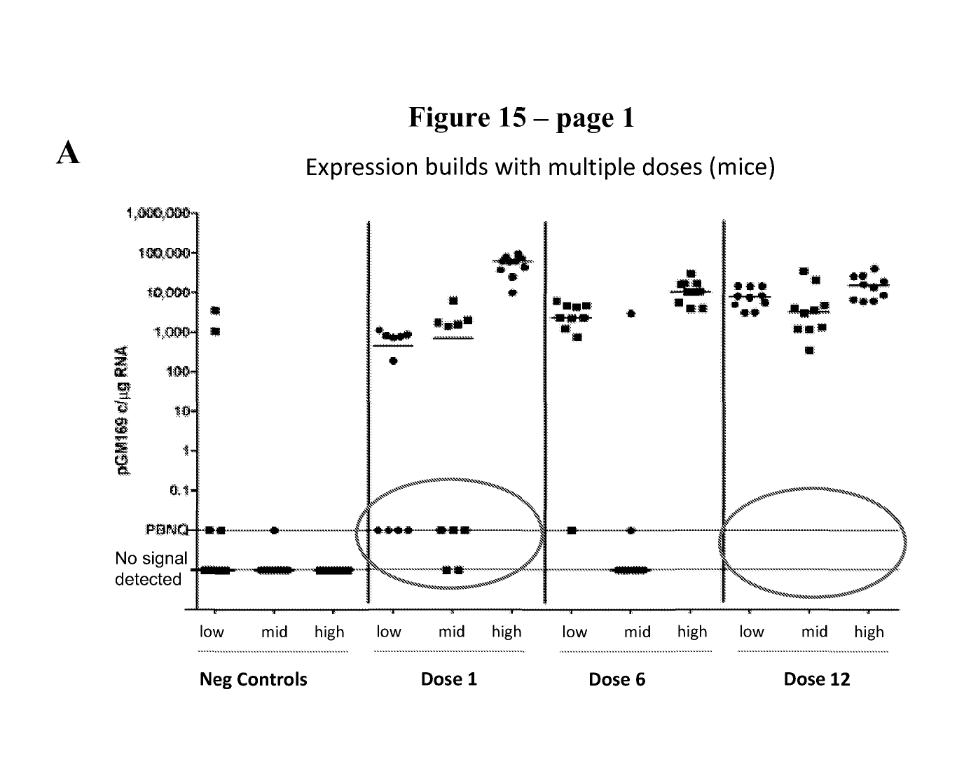

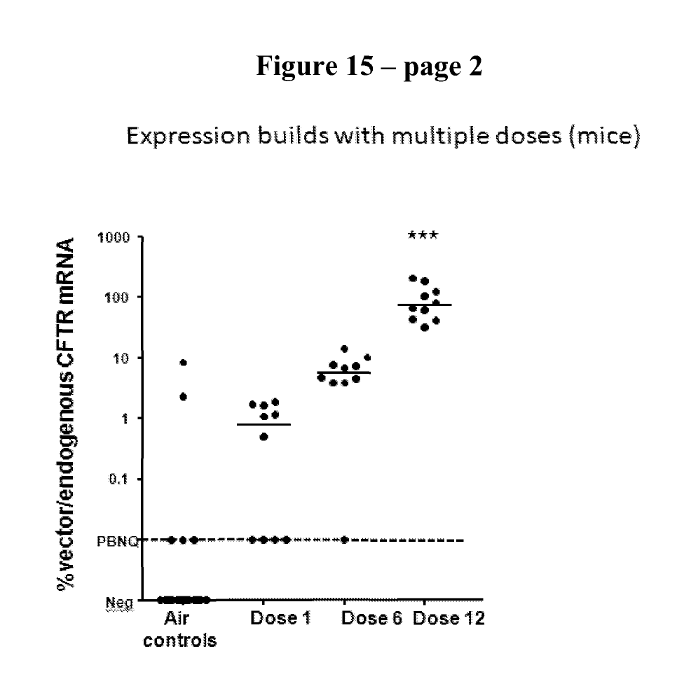

FIG. 15: Expression consistence of pGM169 builds with multiple doses

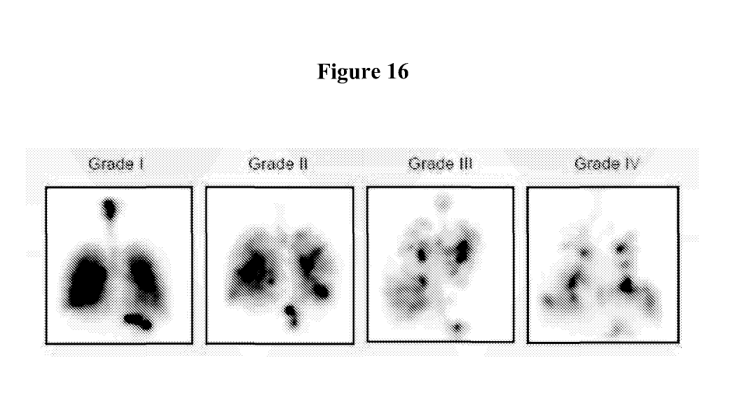

FIG. 16: ^{99m}Tc-labelled human serum albumin was inhaled via a Pari LC nebuliser & Akita system to enhance conducting airway deposition. Anterior and posterior planar gamma camera images and SPECT were used to assess 3-D deposition. Images were scored digitally and, after coding, they were visually graded I-IV by a group of respiratory physicians as follows: I—no defects; II—patchy deposition; III—patchy deposition with large defects; IV—grossly abnormal.

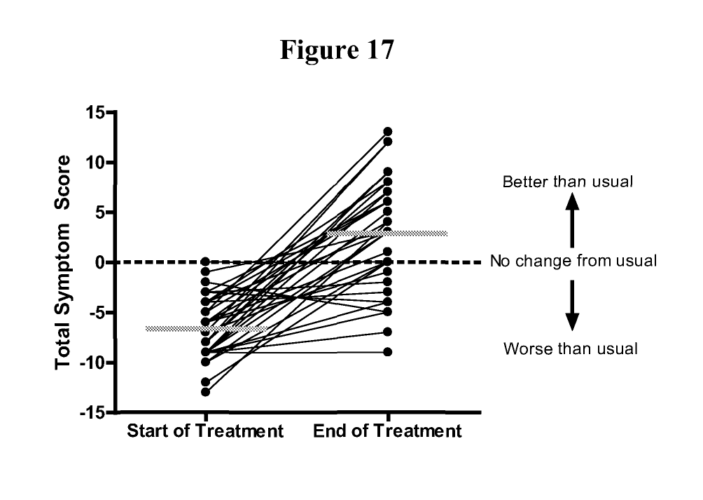

FIG. 17: Effect of antibiotics on total symptom score. Each pair of points represents a single subject. Horizontal grey lines represent group means.

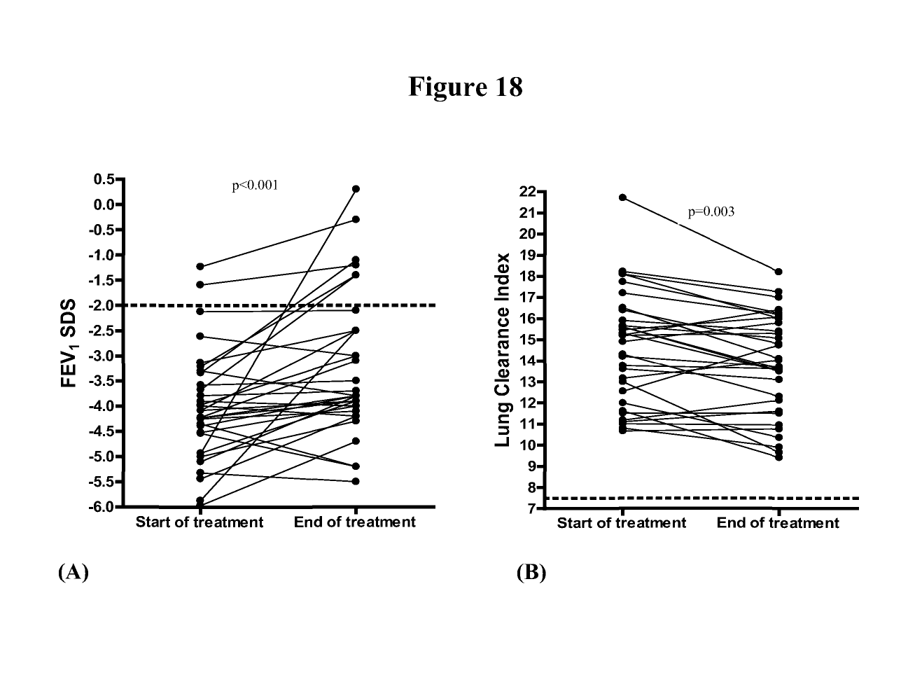

FIG. 18: (a) Change in FEV₁ with treatment of a CF exacerbation. FEV₁ is expressed as standard deviation scores (SDS); anything greater than −2 (horizontal dotted line) is considered to be within the normal range. (b) Change in LCI with treatment of a CF exacerbation. The horizontal dotted line represents the upper limit of normal LCI in a healthy control population (Horsley, A. R., et al., Lung clearance index is a sensitive, repeatable and practical measure of airways disease in adults with cystic fibrosis. Thorax, 2008. 63(2): p. 135-4)

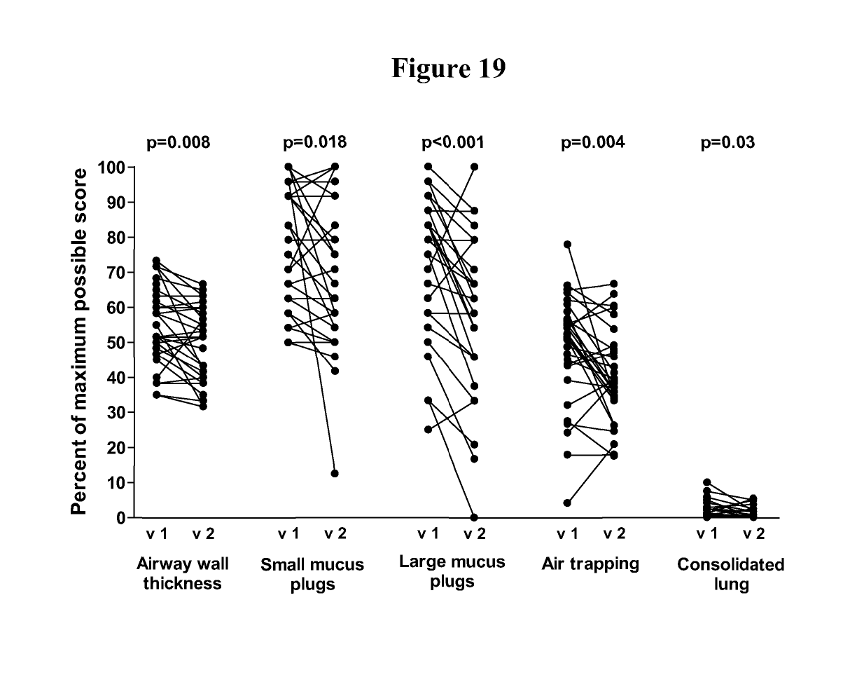

FIG. 19: Change in features of CF lung disease at CT with treatment of a CF exacerbation.

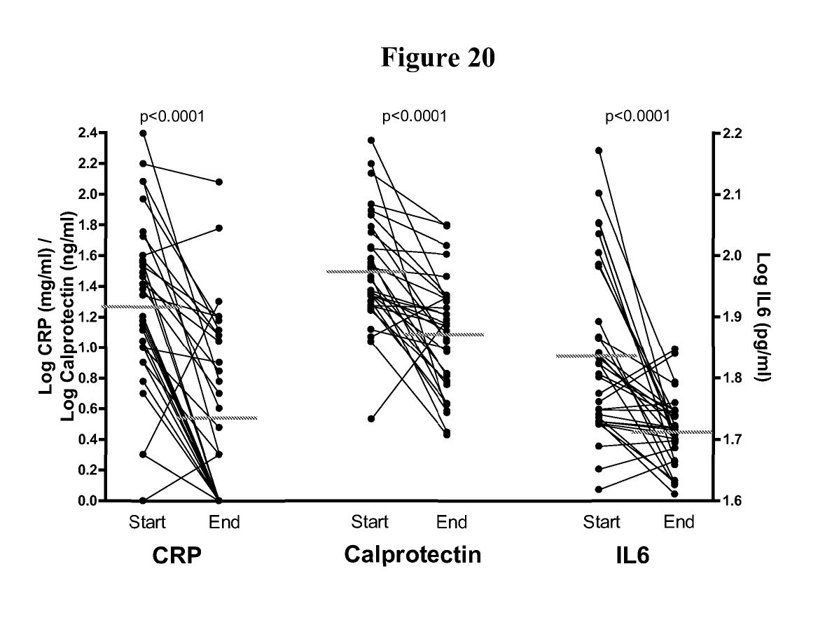

FIG. 20: Change in serum inflammatory markers in CF patients treated for an exacerbation. Each pair of points represents a single patient before and after treatment with intravenous antibiotics. Group means are shown as horizontal grey bars.

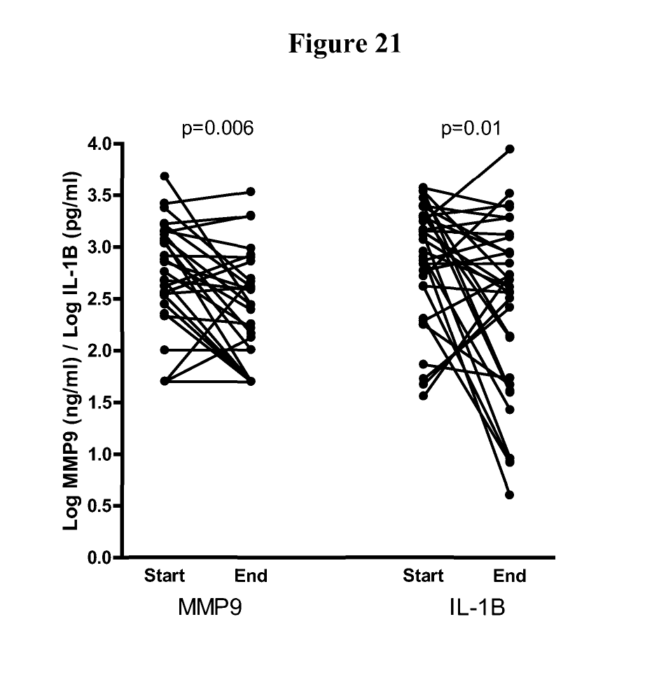

FIG. 21: Change in sputum MMP9 and IL-1β in CF patients treated for an exacerbation. Each pair of points represents a single patient before and after treatment with intravenous antibiotics.

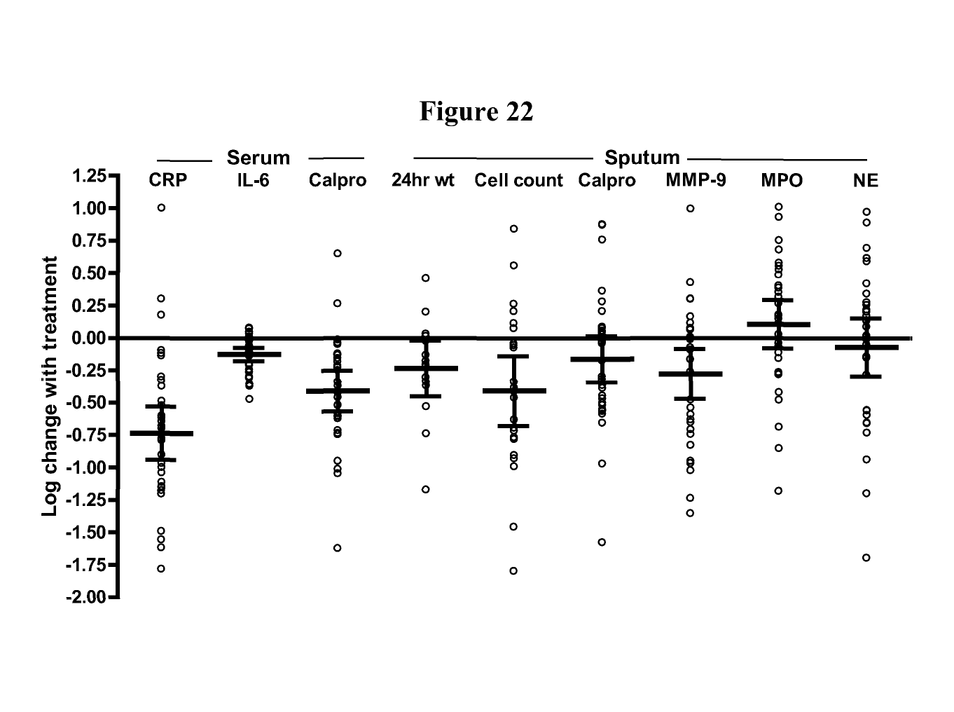

FIG. 22: Log change in serum and sputum inflammatory markers with treatment of an exacerbation. Each point represents change in a single patient, error bars illustrate group mean and 95% confidence interval. Individual and mean values less than zero represent a reduction in the marker with treatment. NE: neutrophil elastase. Not sure we need this as well as other graphs.

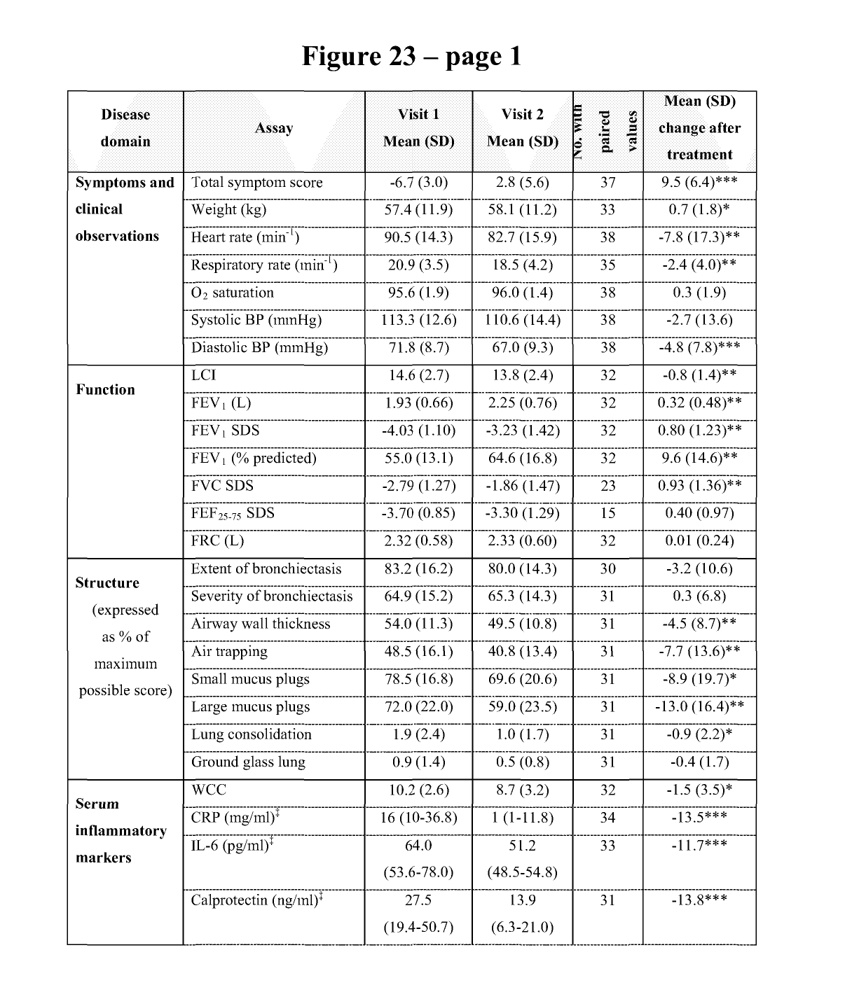

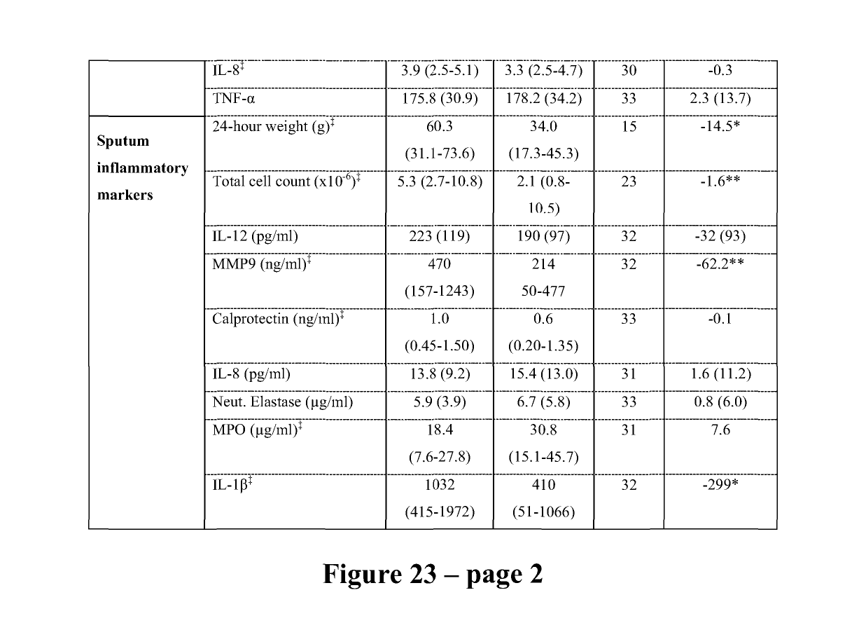

FIG. 23: Summary of changes after antibiotic treatment ‡ Statistics performed on log transformed data, these data quoted as median (IQ range), and median change

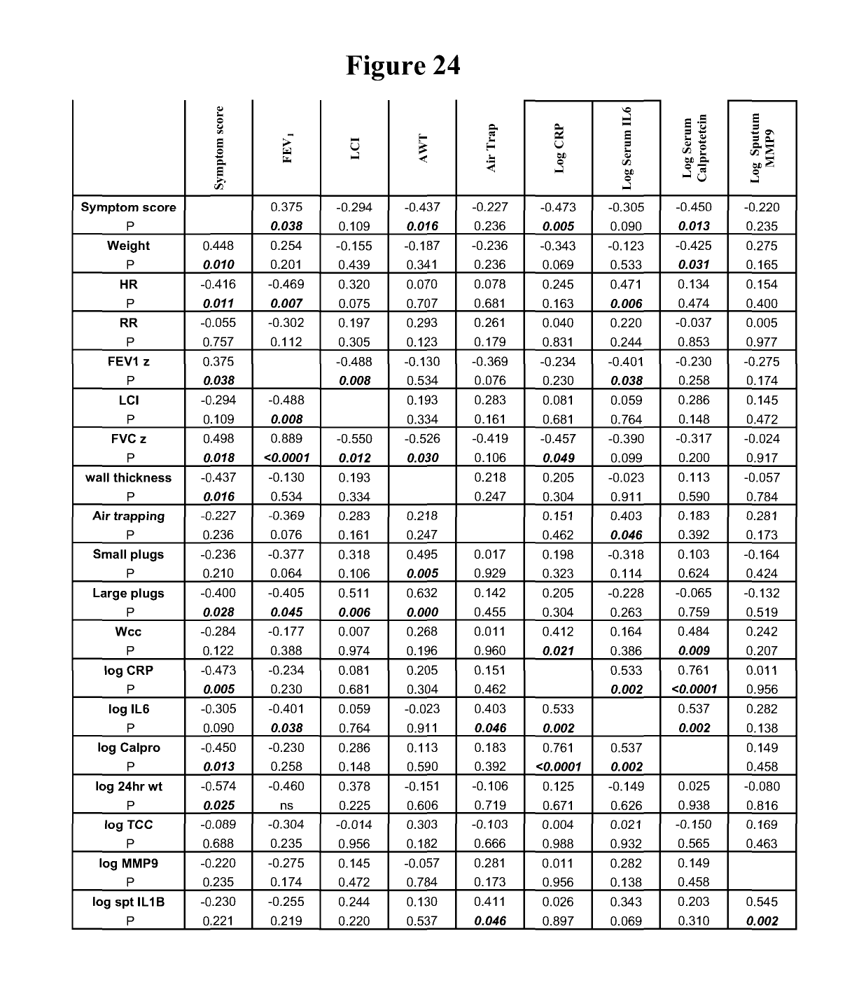

FIG. 24: Correlation mileage chart of changes in markers of disease activity over the course of treatment for an exacerbation of CF lung disease. Correlations are shown between changes in markers with significant change, against selected markers with the most significant change and clinical relevance.

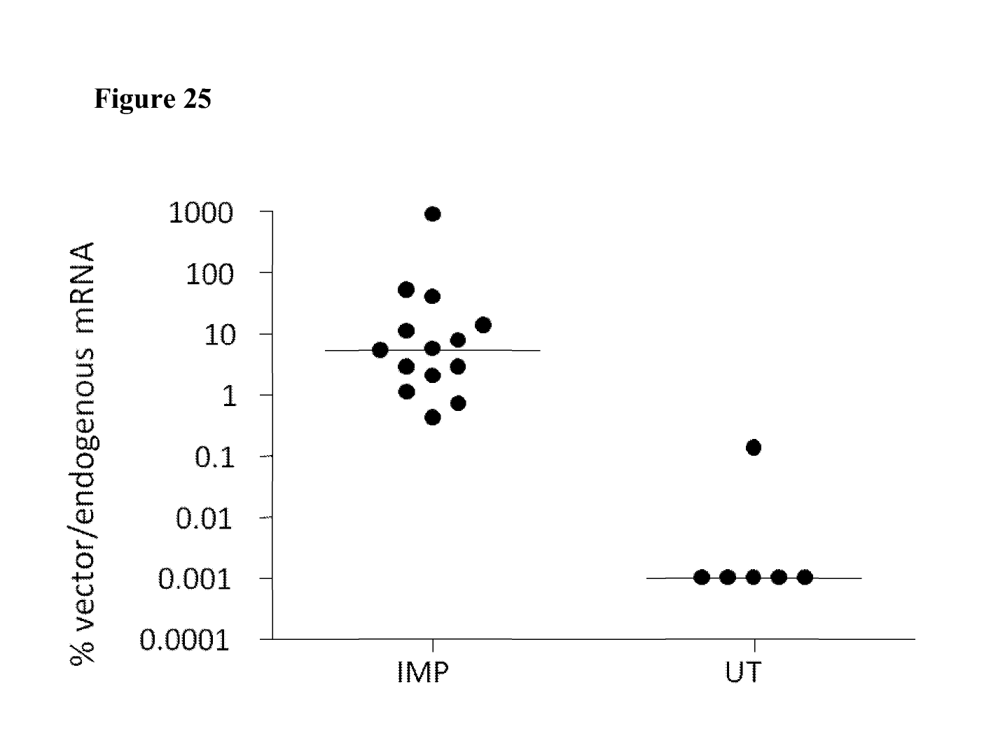

FIG. 25: Vector-specific CFTR expression after GL67A/pGM169 transfection ratioed to endogenous CFTR expression in fully differentiated human air liquid interface cultures (ALIs). ALIs were transfected with GL67A/pGM169, the investigational medicinal production (IMP) or remained untransfected (UT). 24 hrs post-transfection vector-specific and endogenous CFTR mRNA were quantified using quantitative RT-PCR.

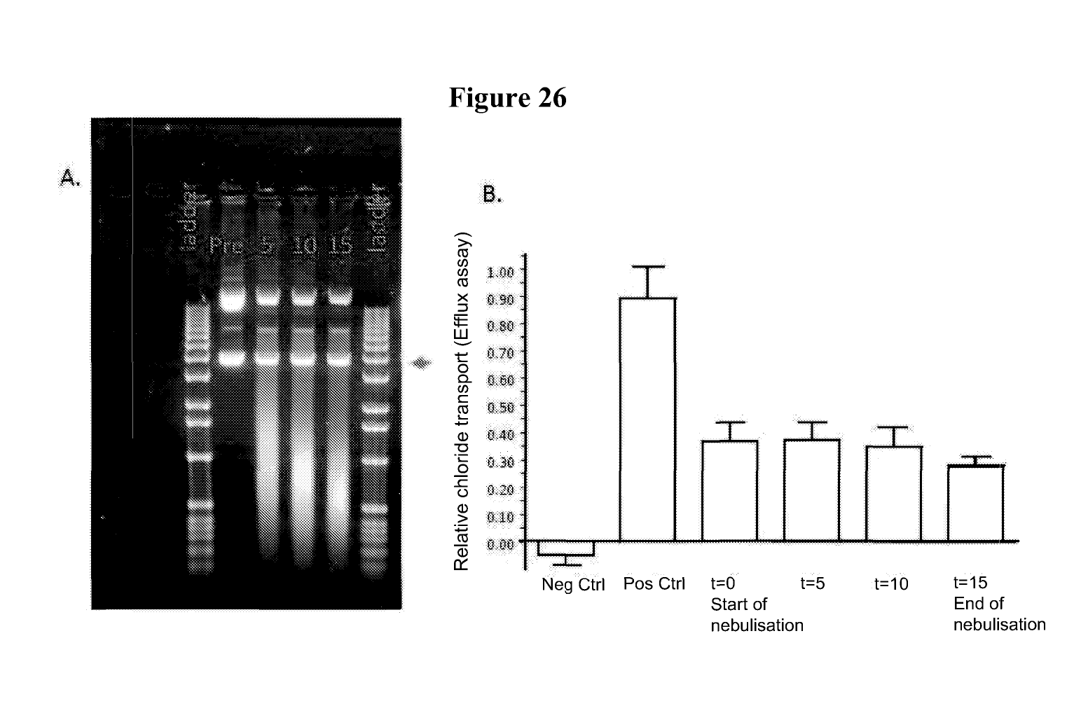

FIG. 26: Integrity of GL67A complexed pGM169 in the AeroEclipse II nebuliser. (A) pGM169 plasmid integrity was assessed by gel electrophoresis pre nebulisation and after 5, 10 and 15 min (end of nebulisation) of nebulisation. Red arrow indicates supercoiled Plasmid DNA. (B) At the same time-point after nebulisation chloride transport mediated by the vector-specific CFTR protein was assessed after in vitro transfection of cells with GL67A/pGM169 complexes collected from AeroEclipse II nebulisers.

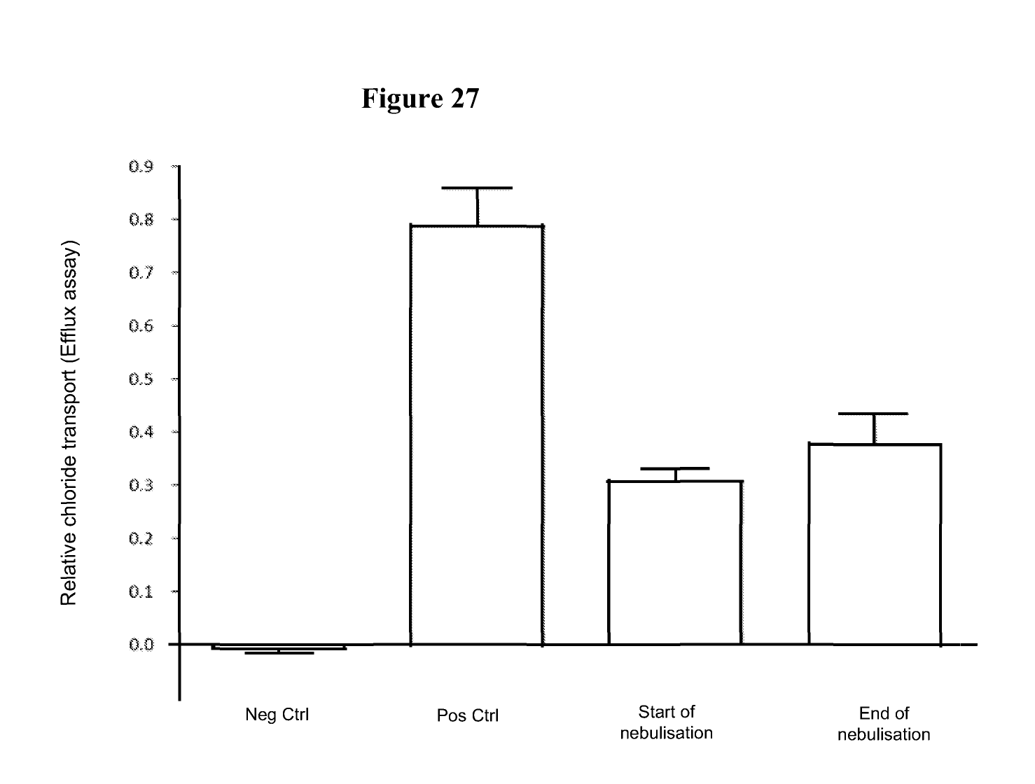

FIG. 27: Assessment of chloride transport mediated by vector-specific CFTR protein after in vitro transfection of cells with GL67A/pGM169 complexes collected from AeroEclipse II nebulisers prior to nebulisation to patients and after transfection with residual complexes left over in the nebulisers at the end of nebulisation.

### EXAMPLE 1

### pGM169 and GL67A

1. pGM169

### A—Overview

A full description of the pGM169 plasmid is provided above in the description. Briefly, it is a covalently closed circular double-stranded plasmid DNA molecule of 6549 base pairs purified from bacteria. It is based on a CpG-free plasmid backbone. The basic features of pGM169 (proceeding clockwise from Obp) are: the CpG-free hCEFI enhancer/promoter; a CpG-free synthetic intron sequence to enhance mRNA splicing; a CpG-free version of the CFTR coding sequence termed soCFTR2; a CpG-free version of the bovine growth hormone polyadenylation sequence; a CpG-free version of the R6K bacterial plasmid origin of replication; a CpG-free version of the kanamycin resistance gene, and a CpG-free synthetic bacterial promoter sequence termed EM7.

The nucleic acid sequence of pGM169 is provided in SEQ ID NO:1 below. A schematic map of the plasmid is provided in FIG. 1.

B—Verification of pGM169 Construction

The completed pGM169 was digested with a variety of restriction enzymes and the resulting DNA fragments were analysed by gel electrophoresis. The resulting fragment sizes matched the sizes calculated from the predicted sequence. The seed stock of pGM169 used to create the GMP master cell bank was completely sequenced with ≧four-fold base redundancy and was found to completely match the predicted sequence. pGM169 isolated from the master cell bank was completely sequenced under GLP conditions and was also found to completely match the predicted sequence.

### C—Transgene Product Analysis

Western blot analysis using anti-CFTR antibody M3A7 (Upstate/Millipore Ltd, Chandlers Ford, UK) of protein extracts from 293T cells transfected with pGM169 demonstrated a protein with the expected molecular weight for CFTR, thus demonstrating correct transcription and translation of the CFTR coding sequence in pGM169. Iodide efflux analysis of 293T cells transfected with pGM169 demonstrated a cAMP-dependent halide efflux with the expected pharmacological activation and kinetic profile for CFTR thus indicating the presence of functional CFTR protein.

D—GMP Manufacture of pGM169

GMP manufacture of pGM169 is conducted by VGXi Inc. (The Woodlands, Texas, USA). Briefly, fermentation and purification of the plasmid are performed in batch mode. Bacteria containing the plasmid are grown from a Master or Working Seed, fermented to high density, and harvested. The bacteria are then lysed to release their contents, including the plasmid, into solution. The lysate is subjected to three significant purification steps: solid/liquid separation, ion exchange chromatography and hydrophobic interaction chromatography. Subsequently, the purified plasmid is concentrated and desalted by ultrafiltration/diafiltration into water for injection and finally subjected to aseptic filtration to provide the bulk drug substance. This bulk is aseptically filled into single unit vials and stored at −80° C. To prepare the final drug substance, single or multiple pooled lots of bulk drug substance are, if necessary, diluted to the desired final desired concentration with sterile water for injection and then filled into 10 mL clear glass vials at a fill level of 5.2±0.2 mL.

### 2. GL67A

A full description of the pGM169 plasmid is provided above in the description. Briefly, the cationic lipid mixture GL67A consists of a mixture of three components—GL67 (structure shown at FIG. 2A), DOPE (structure shown at FIG. 2B) and DMPE-PEG5000 (structure shown at Figure C), formulated at 1:2:0.05 molar ratio.

GMP formulation of GL67A

GL67A is formulated from GMP GL67, DOPE and DMPE-PEG5000 by OctoPlus N.V. (Leiden, Netherlands). Briefly, individual constituents are first dissolved in t-butanol:water (90:10%) and then mixed in appropriate quantities to obtain a GL67 to DOPE to DMPE-PEG5000 molar ratio of 1:2:0.05. After sterile filtration the lipid is dispensed into individual 10 mL glass lyophilisation vials such that each vial contains 39±10 mg GL67, 94±23 mg DOPE, and 18±5 mg DMPE-PEG. The total amount of lipid in a vial is 128 to 172 mg. The vials are freeze-dried under nitrogen gas for approximately 94 hours at temperatures ranging from −50° C. to +10° C. The vials are capped with aluminium crimp caps, coded and stored at −80° C.

Total amount of lipid/ml when reconstituted in water: 24.4-32.8 mg/ml. Total amount of lipid/ml when mixed with plasmid DNA: 12.2-16.4 mg/ml. Total amount of lipid/5 ml of composition: 61-82 mg dose.

### EXAMPLE 2

### Preparation of the pGM169/GL67A Formulation

The following protocol may be used to prepare a pGM169/GL67A gene therapy product which can be used in the aspects of the present invention.

### Hydration of GL67A

The GMP protocol described above in Example 1 provides a vial containing lyophilized 38.75 mg GL67, 93.71 mg DOPE and 17.81 mg DMPE-PEG5000. The following protocol provides a rehydrated vial of GL67A at a concentration of 28.82 mg/ml that can be used in the preparation of the pGM169/GL67A gene therapy product.
1. Place the vial containing GL67A and water (red crimp vial) into a foam rack.
2. Place the foam rack into the VWR-DVX2500 vortexer.
3. Lower the top plate onto the foam rack and tighten screws on both sides.
4. Set to maximum speed (2500 rpm) for 40 min and press run button.
5. After required time unscrew top plate and lift up, remove vial and check that lipid has completely resolved (no white residues on the bottom of the vial, no visible lumps of material in suspension).
6. If the lipid is not dissolved repeat steps 1-3. Set to maximum speed (2500 rpm) for 10 min and press run button.
7. Repeat step 5 and check that lipid has completely resolved (no white residues on the bottom of the vial, no visible lumps of material in suspension).
8. If the lipid is not dissolved repeat steps 1-3. Set to maximum speed (2500 rpm) for 10 min and press run button.
9. Repeat step 5 and check that lipid has completely resolved (no white residues on the bottom of the vial, no visible lumps of material in suspension).
10. If the lipid is not dissolved repeat steps 1-3. Set to maximum speed (2500 rpm) for 10 min and press run button.
11. Repeat step 5 and check that lipid has completely resolved (no white residues on the bottom of the vial, no visible lumps of material in suspension).
12. If the lipid is not dissolved repeat steps 1-3. Set to maximum speed (2500 rpm) for 10 min and press run button.
13. Repeat step 5 and check that lipid has completely resolved (no white residues on the bottom of the vial, no visible lumps of material in suspension).
14. If the lipid is not dissolved repeat steps 1-3. Set to maximum speed (2500 rpm) for 10 min and press run button.
15. Repeat step 5 and check that lipid has completely resolved (no white residues on the bottom of the vial, no visible lumps of material in suspension).
16. If the lipid is not dissolved repeat steps 1-3. Set to maximum speed (2500 rpm) for 10 min and press run button.
17. Repeat step 5 and check that lipid has completely resolved (no white residues on the bottom of the vial, no visible lumps of material in suspension).
18. Discard GL67A, if the total vortexing time exceeds 100 min.
   Equipment and Materials for pGM169/GL67A Formulation
1. A LMD-2 pneumatic mixing device setup and calibrated. This apparatus is described in Davies et al (2010) Biotechniques vol. 49 p 666-668. It is a pneumatic mixing device that can be used for rapid and reproducible production of gene therapy formulations.
2. Dual lumen syringe barrels (Plas-Pak Industries Cat 14B35(S505A)
3. Dual lumen syringe plungers (Plas-Pak Industries Cat 14C35(S446)
4. Eight element static mixers (Plas-Pak Industries Cat 003M08B005-3(S732)
5. 16 G disposable needles with Luer fitting hub (Sigma Cat Z118036)
6. 5 ml sterile disposable syringes with Luer fitting hub
7. Thawed vial of pGM169 plasmid, at a concentration of 5.3±0.3 mg/ml. The GMP protocol described above in Example 1 provides a 10 ml vial of pGM169 plasmid that can be used in this protocol.
8. Thawed and re-suspended vial of GL67A, at a concentration of 28.62 mg/ml.

### Method

The following method can be used to prepare a 10 ml aliquot of pGM169/GL67A formulation.
1. Fully insert a single dual lumen syringe plunger into a single dual lumen syringe barrel.
2. Withdraw plunger until end of plunger approaches the end of the syringe barrels. At this point there will be resistance to any further withdrawal of the plungers and the syringe barrels will be filled with air.
3. Hold dual lumen syringe assembly with the syringe barrels uppermost and the plunger pointing downwards. Support in this inverted position.
4. Attach a 16 G needle to a disposable 5 ml syringe and take up 5 ml of room air into the syringe barrel.
5. Holding a vial of pGM169 upright, insert the needle through the top of the rubber stopper on the vial and inject the 5 ml of air into the vial. Hold the needle firmly in place to resist the increased pressure in the vial trying to push it out.
6. With the syringe now emptied, carefully invert the vial with the needle and syringe still in situ. Keeping the tip of the needle below the surface of the DNA solution, withdraw 5 ml of pGM169 into the syringe.
7. Remove the needle and syringe from the DNA vial and insert into one barrel of the dual lumen syringe assembly via the small hole at the tip of the barrel (see below).
8. Carefully inject 5 ml of pGM169 into the syringe barrel.
9. Remove and discard 5 ml syringe and 16 G needle.
10. Repeat the procedure using a fresh 16 G needle and 5 ml syringe to transfer 5 ml of re-suspended GL67A from the vial to the other barrel on the dual lumen syringe.
11. Whilst keeping the dual lumen syringe inverted, attach the 8 element static mixer to the end of the dual lumen syringe by pushing into place and then rotating the mixer 90° clockwise to lock into position beneath plastic retainers.
12. It is now safe to hold the dual lumen syringe assembly containing 5 ml of pGM169 and 5 ml of GL67A vertically with the static mixer pointing downwards. Some air (1-2 ml) will remain within the syringe barrels. This is normal and important to allow complete emptying of the syringes upon activation of the mixer.
13. Check calibration of LMD-2 is within acceptable limits (see instruction guide)
14. Open the syringe chamber door of the pressurised and calibrated LMD-2 by pulling the top of the anterior clear perspex panel gently forwards—you will hear a small escape of gas as you open the door. This is normal and prevents activation of the device whilst the door is open.
15. Insert the dual syringe assembly into the syringe chamber such that the tip of the static mixer protrudes through the hole at the bottom of the chamber.
16. Close the syringe door by pushing it gently backwards until it clicks into place. The device is now primed and ready for operation.
17. Place a sterile 20 ml Sterilin tube immediately below the protruding static mixer element to collect mixed formulation.
18. Activate pneumatic mixer by turning the switch on the right hand side of the device to the ON position (down). The pneumatic plunger will descend and push the dual plunger of the dual lumen syringe assembly. Mixed pGM169/GL67A complexes will be ejected from the static mixer into the 20 ml Sterilin collection tube.
19. When the dual syringe has been fully depressed, quickly return the switch to the OFF position (up) to return the pneumatic plunger to the “REST” position.
20. Screw cap into place on 20 ml Sterilin tube and store at room temperature for 20 min before use.
21. Open syringe chamber door and remove and discard empty dual lumen syringe. Removal is aided by pushing the static mixer vertically upwards from beneath.
22. Mixer is now ready for next sample.

### EXAMPLE 3

### Optimisation of Nebuliser Parameters for the Clinical Delivery of Liposomal Gene Therapy Formulations for Cystic Fibrosis Gene Therapy

### Summary

The AeroEclipse II nebuliser was selected above other available nebuliser devices for a number of significant reasons. (i) it was capable of nebulising GL67A/pDNA formulations without excessive foaming of the formulation within the unit and concomitant interference with aerosol generation. (ii) The size characteristics of GL67A/pDNA aerosols (MMAD 3.4±0.1 μm, FPF 71.4±1.5%) were appropriate for aerosol delivery to the lungs of CF patients with optimal aerosol characteristics achieved at a nebuliser operating pressure of 50 psi. (iii) Unlike the majority of nebulisers investigated, the AeroEclipse II was CE marked for operation at this relatively high operating pressure. (iv) Although the AeroEclipse II delivered aerosol more slowly (0.17±0.01 ml/min) than alternative devices, the breath actuation mechanism within the device permitted more efficient delivery of formulation to the lungs of patients (oral bioavailability of 83.0±2.3%), with a significant reduction in aerosol wastage and environmental contamination. (v) Importantly, nebulisation of GL67A/pDNA formulations using the AeroEclipse II was not associated with excessive modification of the physical characteristics of the formulation during the nebulisation period (68.5±0.9% of pDNA delivered intact) with aerosol delivery to the lungs of mice resulting in robust gene expression.

### Introduction:

A key challenge to the successful translation of gene therapy to the clinic is the need to tackle the issue of efficient delivery at an early stage in development. For treatment of a range of lung diseases including cystic fibrosis, emphysema and lung cancer, the situation is further complicated by the use of aerosol technology. The relative accessibility of the pulmonary epithelium makes aerosol delivery of gene transfer formulations an attractive possibility. However, development of such aerosol gene therapies has been hampered by difficulties in achieving compatibility between many gene therapy formulations and standard nebuliser devices. Consequently, only eight clinical trials (all in patients with CF) have so far incorporated aerosol delivery of gene transfer agents (GTAs) to the lungs as a key component of the study.

Recent advances in nebuliser design have resulted in a wide variety of clinically approved devices that utilise compressed air, ultrasonic waves or a vibrating mesh to generate aerosol. However, whilst these devices have proven highly successful for the aerosol delivery of small-molecule aqueous pharmaceuticals, the physical characteristics of some gene transfer formulations may impair or prevent aerosol generation altogether. In addition, the susceptibility of many GTAs to the substantial hydrodynamic shear forces generated during aerosol production has limited the development of viable therapies. With the exception of adeno-associated virus (AAV), the majority of viral vectors investigated have demonstrated significant loss of viability following aerosolisation. Recent investigations into the aerosol delivery of the highly efficient lung gene transfer agent Sendai virus (SeV) demonstrated that less than 1% of initial viral activity could be detected following jet nebulisation.

Considerably more success has been achieved with aerosol delivery of nonviral formulations consisting of pDNA complexed to a variety of cationic lipids or polymers. Whilst pDNA itself is extremely shear-sensitive and prone to rapid degradation during nebulisation condensation of the pDNA molecule following complexation with a non-viral gene transfer agent can infer a degree of protection during aerosol generation. Importantly, the level of protection and hence the efficacy of aerolised formulations varies significantly between GTAs and between nebulisers. As a result, development of any viable aerosol gene therapy system is crucially dependent upon the identification of not only a suitable gene transfer agent, but also a compatible nebuliser device for the chosen formulation. Where such compatibility has been achieved, the results have been highly promising and robust gene expression has been observed in vivo following pre-clinical aerosol studies using a number of formulations including polyethylenimine (PEI), nanoparticles and lipid based formulations.

One particularly successful non-viral vector that has been aerosolised to the lungs of patients is the cationic lipid mixture formulation GL67A. Encouraging results from two clinical trials in patients with Cystic Fibrosis were published demonstrating successful aerosol delivery of a single dose of GL67A complexed with pDNA encoding the cystic fibrosis transmembrane conductance regulator (CFTR) gene. In one case the lung delivery of the CFTR showed approximately 25% correction to normal levels. However positive effects were short-lived, probably due to the use of a viral promoter to express the transgene, resulting in promoter attenuation and loss of ongoing transgene expression, compared with mammalian promoters capable of persistent expression such as UbC. Subsequent improvement of the GL67A formulation has been achieved utilising a novel, hybrid CpG-free promoter (hCEFI) in the pDNA, such that when aerosolised to the mouse lung the new GL67A/pGM169 formulation was capable of directing persistent CFTR transgene expression for >x months with minimal inflammation. In order to test the efficacy and safety of GL67A/pGM169 formulations in the clinic, a suitable aerosol delivery methodology is required.

Unfortunately, the equipment used for aerosol delivery of GL67A in earlier clinical trials is no longer available and although considerable pre-clinical development of GL67A aerosol formulations has been achieved using the Pari LC+ jet nebulizer, a practical aerosol delivery protocol for large-scale clinical studies will require a device that fulfils a number of essential criteria. The selected device must not only be capable of retaining the efficacy of the formulation after aerosolisation, but it must also generate aerosol droplets with physical characteristics suitable for deposition to the target areas of the lung. The device must be clinically approved, mechanically robust, easy to use and capable of delivering suitable volumes of formulation to the lungs of patients in a clinically acceptable timeframe. Finally, to maximise the potential clinical benefits of aerosol delivery and simultaneously minimise wastage of expensive gene therapy formulations it is vital that the aerosol delivery device is also as efficient as possible.

Here we combine the methodologies of gene therapy and aerosol science to practically assess the suitability of a wide variety of commercial nebulisers for the clinical delivery of GL67A/pDNA aerosols. Based on the selection criteria outlined above, one nebuliser was subsequently chosen for incorporation into a Phase I clinical study for the treatment of patients with CF. Importantly, the methods and techniques described are directly transferable to alternative gene therapy formulations and lung disease applications.

### Materials and Methods

### Nebuliser Operation

The following nebulisers were investigated for aerosolisation of GL67A/pDNA complexes: Euroneb® (Medikare, Mainz, Germany), eFlow® (Pari GmbH, Starnberg, Germany), I-neb (Philips Respironics UK, Chichester, UK), Sidestream (Philips Respironics), Aerotech II (Biodex Medical Systems Inc, NY, USA), LC+(Pari), Sprint (Pari), Junior (Pari), Star (Pari) and AeroEclipse II (Trudell Medical International, London, ON, Canada). Ultrasonic and vibrating mesh nebulisers were operated in continuous output mode and pneumatic nebulisers were operated using medical air from a compressed air cylinder or using air from two commercially available nebuliser compressors, the TurboBoy S (Pari) or the BoySX (Pari). Operating pressure was measured via an in-line Digitron 2023P digital pressure Meter (Sifam Instruments Ltd, Torquay, UK) connected between gas source and nebuliser.

### Preparation of GL67A/pDNA Formulations

The plasmid pCIKLux (5.6 kb) was used in all studies and contains the firefly luciferase gene under the control of the human cytomegalovirus virus immediate/early promoter/enhancer. Endotoxin-free pDNA was prepared by Bayou Biolabs (Harahan, La., USA). GL67A was formulated by OctoPlus (Leiden, Netherlands) by combining GL67 (Genzyme Corporation, Cambridge, Mass., USA), DOPE (Avanti Polar Lipids, Alabaster, Ala., USA) and DMPE-PEG5000 (Avanti Polar Lipids) at a molar ratio of 1:2:0.05 (GL67:DOPE:DMPE-PEG5000). GL67A/pDNA complexes were prepared at a molar ratio of 6 mM:8 mM as described herein. PEI/pDNA complexes were prepared at a final pDNA concentration of 0.2 mg/ml and a PEI nitrogen (N) to pDNA phosphate (P) ratio of 10:1 as described previously.

### Physical Characterisation of GL67A/pDNA Formulations

Samples were prepared for electron microscopy using a routine negative staining technique. Carbon/formvar coated copper grids were floated on drops of the various suspensions for 30 seconds. The excess fluid was removed and the grids floated on drops of 1% methyl tungstenate, dried and examined in a Jeol 1200EX transmission electron microscope. Particle size and zeta potential of GL67A/pDNA complexes was measured by laser light scattering using a Malvern Zetasizer Nano (Malvern Instruments, Malvern, UK). Five measurements per sample were performed at a fixed temperature of 25° C. and using a viscosity of water value of 0.89 cP. Viscosity measurements were performed using a falling ball viscometer (Gilmont Instruments, Barrington, Ill., USA) according to manufacturers instructions. A minimum of 5 measurements was performed for each sample at a temperature of 21° C. Surface tension measurements were performed at 20° C. using a Du Nouy ring balance and 10 ml of starting sample. All measurements were performed in triplicate.

### Aerosol Size Characterisation

Aerosol size characteristics were determined using a chilled (4-7° C.) Next Generation Pharmaceutical Impactor (NGI) (Copley Scientific Ltd, Nottingham, UK) operating at 15 L/min (±5%) (Berg E, et al. J Aerosol Med. 2007 Summer;20(2):97-104; PMID 17536948) and in accordance with the European Pharmaceutical Aerosol Group (EPAG) proposed monograph (Pharmeuropa Vol. 18, No. 2, April 2006; p281-283) for particle size testing for nebuliser devices under CEN (European Committee for Standardisation) standard EN 13544-1. Briefly, aerosol was collected over a period of 4 minutes with deposited material subsequently eluted from each impactor stage using 10 ml water. For GL67A/pDNA studies the mass of pDNA deposited upon each stage was quantified using the Quant-it Picogreen dsDNA assay kit (Invitrogen, Eugene, Oreg., USA). Salbutamol was quantified by UV spectrophotometric analysis at 224 nm (Lange 2000 AmJResCritCareMed). Aerosol size characteristics including the mass median aerodynamic diameter (MMAD) and fine particle fraction (FPF) were determined from log-probability graphs of cumulative mass fraction versus stage cut-off diameter using CITDAS particle analysis software (Copley Scientific Ltd). Cut-off diameters of 14.1, 8.61, 5.39, 3.29. 2.07, 1.35 and 0.97 μm were utilised for NGI stages 1 to 7 respectively (Marple V A, et al. J Aerosol Med. 2004 Winter;17(4):335-43; PMID 15699634).

### Aerosol Delivery Rates

Assessment of aerosol delivery rate was performed under simulated breathing conditions as recommended in the European Pharmaceutical Aerosol Group (EPAG) proposed monograph (Pharmeuropa Vol. 18, No. 2, April 2006; p280-281) for particle size testing of nebuliser devices under CEN (European Committee for Standardisation) standard EN 13544-1. Nebulisers were attached directly to a Large Animal Volume controlled Ventilator (Harvard Apparatus, Holliston, Mass., USA) with inhaled air entrained directly through the nebuliser device. Aerosol was generated under simulated breathing conditions with a tidal volume of 500 ml, a breathing frequency of 15 breaths per minute and an inhalation/exhalation ratio of 1:1. Rate of aerosol generation was calculated by weighing the nebuliser reservoir at intervals during aerosol delivery. Nebulisers were run continuously until no further aerosol production was observed and the dead volume of the device was determined based on the mass of formulation remaining in the reservoir. A minimum of 3 separate assessments were performed for each nebulizer operating condition. Nebulisers were single use with a new nebuliser used for each measurement.

### Aerosol Delivery Efficiency

Assessment of oral delivery efficiency was performed by inclusion of aerosol collection filters (Pari) on the inspiratory and expiratory arms of the breathing circuit (FIG. 4c). Nebulisers were run for 1-3 mins with collection filters in situ before the amount of lipid deposited on each filter was quantified using a modified fluorescamine assay (Ferrari 98). Briefly, lipid was extracted by vortexing collection filters for 30 s in 20 ml of RLT buffer (Qiagen Ltd, Crawley, UK) before appropriate dilutions were prepared in sterile water. A volume of 100 μl of lipid sample was mixed with 900 μl of a 150 μM fluorescamine solution and fluorescence at 480 nm measured following excitation at 392 nm using a Gemini XPS fluorescent plate reader (Molecular Devices, Sunnyvale, Calif., USA). Collected samples were quantified relative to standard curves prepared from the initial aerosol formulation Inhalation efficiency was defined as the proportion of total collected aerosol (inhalation+expiration filters) isolated from the inhalation filter.

### Quantification of GL67A/pDNA Components During Nebulisation

To facilitate measurement of lipid and pDNA concentration in nebulizer reservoirs and aerosol, each component was fluorescently labelled prior to complexation. GL67A was supplemented with carboxyfluorescein-labelled DOPE (Ext 488 nm, Ems 515 nm) (Avanti) at 0.1% of standard DOPE levels and pDNA was labelled by addition of trace amounts of the fluorescent DNA probe POPO-1 (Ext 434 nm, Ems 456 nm) (Molecular Probes, Eugene, Oreg., USA) sufficient to label 1 in 1600 nucleotides. Dual labelled complexes were prepared at a molar ratio of 6 mM:8 mM and loaded into the nebulizer reservoir. Aerosol was generated under simulated breathing conditions as described above and generated aerosol was captured via inertial impaction upon the walls of a Dreschel bottle connected in-line between the nebulizer and the ventilator. At intervals during the nebulisation procedure samples of material remaining in the nebuliser reservoir or collected from the aerosol were removed and the concentration of pDNA and lipid assayed using a Gemini XPS fluorescent plate reader (Molecular Devices). Collected samples were quantified relative to standard curves prepared from the initial fluorescently labelled aerosol formulation.

### Gel Analysis and Densitometry

The pDNA component of collected GL67A/pDNA samples was isolated using phenol/chloroform extraction to remove lipids. Briefly, 75 μl of collected sample was vortexed for 10 s with 500 μl of phenol/chloroform (Ambion, Austin, Tex., USA) before centrifugation at 16,000 g for 5 min. The aqueous phase incorporating isolated pDNA was removed and equivalent ng loads of each sample were size-fractionated by electrophresis at 2-10 V/cm through a 0.7% agarose gel containing 0.5 μg/ml ethidium bromide. For quantification of plasmid degradation in the nebuliser reservoir, the intensity of bands corresponding to covalently closed circular pDNA was measured using the software program NIH Image 1.62 (http://rsb:info.nih.gov/nih-image/) as described previously (Davies L A, et al. Pharm Res. 2005 August; 22(8):1294-304. Epub 2005 Aug. 3; PMID 16078139). The amount of supercoiled plasmid present in samples taken prior to nebulisation was arbitrarily designated as 100% and the amount remaining at time-points during nebulisation was expressed as a percentage of this value. Estimation of total plasmid dose delivered without degradation was performed using Area Under the Curve (AUC) analysis of individual plasmid degradation curves using GraphPad Prism software v5 (GraphPad, San Diego, Calif.).

### In Vivo Aerosol Delivery

All research involving the use of animals adhered to the United Kingdom Home Office Animals (Scientific Procedures) Act 1986. Aerosol delivery of GL67A/pDNA formulations was performed using a continuous, unrestrained whole body exposure protocol. Mice (n=6-24) were placed into an 8 L Perspex exposure chamber and exposed to aerosol generated using nebulisers operating at 50 psi and with compressed air as the driving gas (BOC, Bristol, UK). Total lung luciferase was assayed in all animals 24 hrs following aerosol administration as described previously (Davies L A, et al. J. Gene Medicine 2007 March; 9:184-96; PMID 17351986).

### Statistical Analysis

Error bars on graphed data represent mean±standard error of the mean (SEM) for all data sets. Multiple groups were analysed using one-way ANOVA followed by Dunnett's multiple comparison test. Data were considered to be significantly different when p<0.05 (*p<0.05, **p<0.01 and ***p<0.001). GraphPad Prism software v5 (GraphPad, San Diego, Calif.) was used for the analyses.

### Results

### Physical Characteristics of GL67A/pDNA Complexes

To investigate the physical characteristics of GL67A/pDNA formulations, complexes were prepared at a previously optimised molar ratio of 6 mM:8 mM and the viscosity, surface tension and particle size were measured (Table 2). For comparative purposes, the physical characteristics of an alternative aerosol gene therapy formulation (pDNA complexed to 25 kDa PEI) and a more “standard” pharmaceutical aerosol consisting of a 1% (w/v) salbutamol in 0.9% NaCl solution were also analysed. Whilst PEI/pDNA and salbutamol formulations demonstrated similar physical properties to water, GL67A/pDNA formulations were significantly (p<0.001 ANOVA+Dunnett's multiple comparison test) more viscous (6.32±0.09 cP) and demonstrated a significantly lower (p<0.001) surface tension (42.2±0.3 mN/m) than the other formulations (Table 2). In addition, particle size analysis revealed GL67A/pDNA formulations contained relatively large particles (930±130 nm) compared to PEI/pDNA complexes (75±4 nm). Further analysis by electron microscopy suggested that the observed GL67A/pDNA particulates resulted from the aggregation of smaller (approx 200 nm) multi-lamellar lipoplexes formed during the complexation process (FIG. 3a).

### Performance of GL67A/pDNA Complexes in Nebulisers

To determine if stable GL67A/pDNA aerosols could be generated using commercially available nebuliser devices, complexes were loaded into the reservoirs of one ultrasonic, 2 vibrating mesh and 7 pneumatic nebulisers using half the maximum fill volume recommended by the manufacturer (Table 3). Nebulisers were operated according to manufacturers' instructions and the ability to generate stable aerosols was recorded. All nebulisers generated stable aerosols using PEI/pDNA or a standard 1% salbutamol solution. With GL67A/pDNA formulations, no aerosol was generated using the ultrasonic nebuliser and with the vibrating mesh nebulisers, a small amount of aerosol was observed initially but output rapidly decreased and stopped after a few seconds of operation. In contrast, considerable aerosol output was observed with all pneumatic nebulisers tested, although in both the Aerotech II and Up-Mist nebulisers, aerosol production was repeatedly interrupted by the tendency of GL67A/pDNA formulations to “foam” and splutter out of the nebuliser resulting in considerable loss of material. Although moderate foaming was also observed in the remaining pneumatic nebulisers, the associated reservoir geometries prevented loss of material and stable aerosol production was achieved.

### Aerosol Size Characterisation

To assess the potential size characteristics of GL67A/pDNA aerosols, complexes were nebulised using the Pari LC+ nebuliser and the resultant aerosol analysed by cascade impaction using a chilled Next Generation pharmaceutical Impactor (NGI). Aerosols were generated over a range of operating pressures and the Mass Median Aerosol Diameters (MMADs) of resultant aerosols compared to those generated using a standard 1% salbutamol solution. MMAD for both formulations was inversely proportional to the nebuliser operating pressure (Linear regression analysis with R2 values of 0.94 and 0.71 for GL67A/pDNA and salbutamol respectively) with smaller aerosol droplets produced at higher operating pressures (FIG. 3b). Using the manufacturers recommended operating pressure of 29 psi, the measured MMAD of salbutamol formulations (4.0±0.2 μm) was consistent with published literature (Pari instruction leaflet). However, under identical operating conditions, GL67A/pDNA formulations were associated with the generation of much larger droplets (5.5±0.2 μm) and after controlling for the effect of nebuliser operating pressure a significant difference was observed in the MMADs achieved with salbutamol and GL67A/pDNA formulations (p<0.001, ANCOVA). Droplets larger than 5 μm are generally considered too large for respiratory delivery under normal breathing conditions (ref) and so to optimise nebuliser parameters for the generation of suitable GL67A/pDNA aerosols for delivery to patients, the MMAD and fine particle fraction (FPF) (defined as the proportion of aerosol contained in droplets <5 μm in diameter) of GL67A/pDNA aerosols generated by a variety of nebulisers were determined. Five jet nebulisers were assessed at operating pressures of 29 psi and 50 psi using compressed air from a gas cylinder for aerosol generation. In addition, all nebulisers were also assessed using 2 portable medical compressors (TurboBoy S and Boy SX, Pari). For each nebulizer examined increasing the operating pressure was associated with a reduction in MMAD and an increase in the proportion of respirable GL67A/pDNA aerosol generated (FIG. 3c,d). The largest droplets were observed for each nebuliser when using the portable TurboBoy S and Box SX compressor systems (nominal operating pressures of 17 psi and 22 psi respectively). Conversely, the smallest droplets and hence the greatest proportion of respirable aerosol was generated for each nebuliser when operating at 50 psi. Although 50 psi is in excess of the maximum recommended operating pressure for all but the AeroEclipse II nebuliser (Table 3), under these conditions the Junior (MMAD 3.4±0.1 μm, FPF 74.9±1.4%), Star (MMAD 3.4±0.1 μm, FPF 78.3±1.6%) and AeroEclipse II (MMAD 3.4±0.1 μm, FPF 71.4±1.5%) nebulisers all generated GL67A/pDNA aerosols significantly smaller (p<0.01 ANOVA and Dunnett's post-hoc analysis) and more appropriate for clinical delivery than the LC+(MMAD 4.3±0.4 μm, FPF 60.9±5.8%) used in previous clinical studies (refs). During the course of aerosol size characterisation studies it was noted that the Star nebuliser was susceptible to intermittent spluttering when formulations were aerosolized over extended periods. As a result of encouraging aerosol size data and stability of GL67A/pDNA aerosols over a range of operating conditions, the Pari Junior and AeroEclipse II nebulisers were selected for further investigation. The Pari LC+ was also included for comparison to previous nebulisation studies.

### Nebuliser Output Rate

Protocols for aerosol gene therapy will require delivery of a potentially therapeutic dose to the lung over a clinically acceptable time frame and delivery rate of any device is a key determinant in suitability for clinical studies. However, as aerosol output from many nebuliser designs varies throughout the respiratory cycle it is essential that aerosol output rate is determined under simulated breathing conditions. Breath-enhanced nebulisers minimise aerosol losses by reducing aerosol output during patient exhalation whilst breath-actuated nebulisers such as the AeroEclipse II produce aerosol only during the inspiratory phase. To accurately determine aerosol output rates for GL67A/pDNA formulations under simulated breathing conditions the Junior, AeroEclipse II and LC+ nebulisers were connected to a ventilator set to mimic sinusoidal human breathing at 15 breaths/minute, 1:1 inspiration:expiration ratio and a tidal volume of 500 ml. Nebulisers were operated continuously until no more aerosol was generated and the total aerosol output was measured throughout by determining the weight of material remaining in the nebuliser reservoir. At 50 psi, aerosol output rates for the LC+(0.38±0.01 ml/min) and Junior nebulisers (0.34±0.01 ml/min) were similar but the AeroEclipse II was considerably slower (0.17±0.01 ml/min) (FIG. 4a). In the context of an uninterrupted clinical aerosol delivery study these rates would equate to minimum delivery times of 26 min (LC+), 29 min (Junior) and 59 min (AeroEclipse II) for a nominal 10 ml aerosol dose (Table 4). In contrast, aerosol delivery using the Pari BoySX compressor was significantly slower for each nebuliser at 0.25±0.01 ml/min (LC+), 0.15±0.01 ml/min (Junior) and 0.11±0.01 ml/min (AeroEclipse II) (FIG. 4b) requiring 40, 67, and 90 mins for delivery of 10 ml. Although nebulisers were initially filled to capacity with GL67A/pDNA, a quantity of GL67A/pDNA remained in the reservoir of each device after aerosol generation had ceased. This “dead” volume varied between nebulisers (Table 3) but amounted to approximately 5-10% of the initial reservoir load.

### Delivery Efficiency

Compared to alternative modes of vector administration, aerosol delivery is commonly regarded as a highly inefficient process with significant losses of formulation within the delivery apparatus or lost to the atmosphere. Given the relative inefficiency of non-viral GTAs and the high cost of clinical formulations it is essential that wherever possible, aerosol wastage is minimised. To assess the aerosol delivery efficiency of the LC+, Junior and AeroEclipse II nebulisers, the oral bioavailability (percentage of generated aerosol delivered to the mouthpiece) of GL67A/pDNA aerosols was measured. Aerosol collection filters were introduced on the inspiratory and expiratory arms of the breathing circuit (FIG. 4c) to capture inhaled aerosol and aerosol that would normally bypass the patient and be lost to the environment. Comparative analysis of aerosol deposition on the 2 filters allowed the delivery efficiency of a nebuliser to be accurately assessed under simulated breathing conditions. As would be expected for breath-enhanced nebulisers that increase the proportion of aerosol generated during patient inhalation, the LC+ and Junior nebulisers demonstrated relatively high oral bioavailability of around 65-70% when used in conjunction with the Boy SX compressor (FIG. 4d). However, when operated at 50 psi there was a significant (p<0.01 t-test) fall in delivery efficiency to around 55% in both devices. The observed fall in delivery efficiency may to some degree be explained by a failure of the breath enhancement mechanism in these devices when operated at pressures in excess of the manufacturers' guidelines (Table 3). DNA delivery rates were also different: Junior 882±15 μg/min; AeroEclipse II 438±16 μg/min. The breath-actuated AeroEclipse II nebuliser generates no aerosol during patient exhalation and demonstrated the highest delivery efficiencies with around 83% of aerosolized material collected on the inspiratory filter using the Boy SX compressor. In contrast to the other nebulisers tested, no loss of delivery efficiency was observed at the higher 50 psi delivery pressure (p=0.86 t-test).

### Stability of GL67A/pDNA Formulations During Nebulisation

Jet nebulisers have been shown to concentrate drugs in the nebulizer reservoir over time due to the preferential aerosolisation of the formulation solvent. Such increase in formulation concentration over time impacts the rate of drug delivery to patients but may also result in precipitation of colloidal suspensions such as GL67A/pDNA if excessive concentrations are achieved. In addition, the ratio of lipid and pDNA components in GL67A/pDNA formulations is a key determinant of formulation efficacy and any propensity for differential concentration of components during nebulisation could dramatically impact formulation viability. To investigate the effects of nebulisation upon the GL67A/pDNA formulations, the concentration of the individual components were measured in the reservoir and in the generated aerosol of the LC+, Junior and AeroEclipse II nebulisers over time. Aerosolisation of GL67A/pDNA complexes with all 3 nebulisers was associated with a steady increase in the concentration of both pDNA (FIG. 5a) and lipid (FIG. 5b) in the reservoir over time. However, the rate of concentration increased significantly after 80% of the reservoir volume had been aerosolised, most likely due to the increased rate of recycling of material as the devices emptied. The concentration of pDNA in nebuliser reservoirs increased from a starting concentration of 8 mM to a maximum of 18-24 mM observed in the final 10% of formulation remaining in the nebuliser reservoirs. Lipid concentration over the same time period increased from 6 mM to a maximum of 15-17 mM. Crucially, the ratio of lipid:pDNA did not change over time either in the nebuliser reservoir (FIG. 5c) or in the generated aerosol (FIG. 5d) indicating that neither component was aerosolised preferentially. A small increase in the lipid:pDNA ratio was observed in the reservoir of the Junior nebuliser although significantly, this was not recapitulated in the generated aerosol samples. No evidence of precipitation or formulation aggregation was observed even in highly concentrated samples retained in the nebuliser dead volume.

Shear Related pDNA Degradation

To investigate the effects of jet nebulisation on the pDNA component of GL67A/pDNA complexes, formulations were aerosolised using the LC+, Junior and AeroEclipse II operating at 50 psi. At intervals during the nebulisation process samples of formulation were removed from the nebulizer reservoir (FIG. 6a) or collected from the nebuliser aerosol (FIG. 6b) and the structural integrity of the pDNA component analysed by gel electrophoresis. Results were very similar for both reservoir and aerosol samples. Nebulisation in all 3 devices was associated with a progressive degradation of pDNA indicated by loss of the biologically active covalently closed circular (ccc) and open circular (oc) plasmid forms along with appearance of a low molecular weight smear on the gel indicative of sheared plasmid fragments. As has been observed previously, complexation of pDNA with GL67A offers incomplete protection from shear related degradation during jet nebulisation. However, quantification of pDNA degradation using densitometry (FIG. 6C) demonstrated that the rate of pDNA destruction was identical between the three nebulisers (p>0.05 ANCOVA) and estimation of the proportion of total pDNA dose delivered intact over the course of the aerosol revealed no significant difference (p=0.9 ANOVA) between the LC+(67.7±5.7%), Junior (66.2±1%) and AeroEclipse II (68.5±0.9%) nebulisers.

### In Vivo Testing

Assessment of physical stability of GL67A/pDNA formulations during nebulisation can provide an indication of compatibility between nebuliser and formulation but a final assessment of performance can only be achieved following aerosol delivery and gene expression in vivo. To assess the viability of formulations following aerosol delivery using the LC+, Junior and AeroEclipse II, female BALB/c mice were exposed to 10 ml aerosols of GL67A/pCIKLux generated using each of the nebulisers and luciferase expression in whole lung homogenates was analysed 24 hrs later. All treated mice demonstrated robust luciferase expression above background levels observed in naïve mice (FIG. 7a) but expression following aerosol delivery using the Junior (73.1±7.9 RLU/mg) and AeroEclipse II (87.7±12.1 RLU/mg) nebulisers was significantly higher (p<0.001, ANOVA+Tukeys's post hoc analysis) than that observed with the LC+(13.7±3.6 RLU/mg). The higher gene expression observed in mice using the Junior and AeroEclipse II can be explained at least in part, by the lower MMAD of the generated aerosols (FIG. 3c) which are more suitable for inhalation and deposition in the murine lung (Raabe). Subsequent studies looking at delivery of increasing volumes of GL67A/pCIKLux using the AeroEclipse II nebuliser (FIG. 7b) demonstrated that lung gene expression was dose dependent (Linear regression analysis with R2 value of 0.87) and higher gene expression was achievable by administering multiple reservoir volumes sequentially.

Selection of a Clinical Device

Following detailed assessment of the delivery characteristics of a number of aerosol devices (Table 4), the AeroEclipse II nebuliser was selected for inclusion in a Phase I clinical study for the delivery of GL67A/pGM169 aerosols to the lungs of CF patients. GL67A/pDNA aerosols generated by the AeroEclipse II demonstrated appropriate physical characteristics for lung delivery and the combination of a high respirable fraction of generated aerosol and the high delivery efficiency associated with breath actuation resulted in the highest estimation of lung delivery using this device (Table 4). Similar levels of deposition were indicated using the AeroEclipse II operating at 50 psi from a compressed air cylinder or using the portable Pari BoySX compressor. However, despite the huge practical advantages of using the portable compressor in a large-scale clinical trial, it was decided that a 50 psi operating source would be utilised due to the savings in aerosol delivery time required for individual patients. Importantly, the AeroEclipse II nebuliser has been approved for clinical operation at 50 psi and as such raises no additional safety concerns for operators or patients.

### Discussion

GL67A represents one of the most promising GTAs available for aerosol gene therapy and is currently being investigated for a clinical study for the treatment of cystic fibrosis lung disease. Considerable development and optimisation of the formulation over the last 15 years has resulted in a potent aerosol GTA that has demonstrated robust gene expression following aerosol delivery in a range of animal models including mice, sheep and rhesus macaques. However, aerosol delivery requirements for clinical application of gene therapy formulations are considerably more stringent than those required for validation studies in animals and although the formulation has already been aerosolised to CF patients, the final study took place in the late 1990s and developments in aerosol science now enable a more detailed assessment of aerosol formulations before entry into the clinic. In addition, advances in nebuliser design potentially offer advantages over earlier delivery techniques and may prove beneficial both in terms of patient interface and clinical outcome. Some of the biggest advances have been in the development of nebulisers based on vibrating mesh technology. Being simple to use and capable of delivering drugs more rapidly than other devices they have proven highly popular with clinicians and patients alike. Unfortunately, although both the eFlow and I-neb devices tested were capable of generating excellent aerosols using a 1% salbutamol solution or a PEI/pDNA based gene therapy formulation, neither was capable aerosolizing GL67A/pDNA formulations. The most likely explanation for this failure is the rather atypical physicochemical properties of the GL67A/pDNA formulation itself (Table 2). Optimisation of the formulation to enhance aerosol stability and increase pDNA concentration in the aerosol has resulted in a viscous formulation containing relatively large aggregates of lipid:pDNA complexes. Whilst the aerosol generation process in jet nebulisers is relatively unaffected by the physical properties of the formulation, operation of both ultrasonic and vibrating mesh nebulisers is severely impaired by excessively viscous formulations (Ghazanfari). It is also likely that aerosol generation using vibrating mesh nebulisers was further impaired by the presence of large (˜900 nm) GL67A/pDNA aggregates that may have impeded liquid transfer through the micron sized pores utilised in such devices for aerosol production. Based upon these results it would appear that vibrating mesh nebulisers are inappropriate for the aerosol delivery of concentrated GL67A/pDNA formulations. However, the devices remain an attractive alternative to jet nebulisation for the aerosol delivery of gene therapy formulations with more appropriate physical characteristics.

In contrast to other nebuliser designs, aerosolisation of GL67A/pDNA formulations using jet nebulisers proved relatively straightforward. However, even with jet nebulisers, stable aerosol generation was only possible with a sub-set of devices (Table 3). Due to the high concentration of surface-active agents within the GL67A formulation, there is a propensity for the formulation to foam when exposed to the constant recirculation of material within a nebuliser reservoir. Such foaming is inconsequential if the geometry of the reservoir prevents loss of material into the aerosol stream but can prove catastrophic in devices where loss of material is not prevented. Consequently, many of the nebulisers identified as suitable for stable aerosolisation of GL67A/pDNA were very similar in terms of overall design and geometry. Although similar in design, the physical characteristics of GL67A/pDNA aerosols generated by the different nebulisers were extremely varied (FIG. 3c,d)—highlighting the necessity for independent aerosol characterisation for any prospective aerosol gene therapy formulation/nebuliser combination. In general, aerosols generated using GL67A/pDNA contained significantly larger droplets than would be achieved using more standard aerosol formulations (FIG. 3b) and under many of the conditions tested the GL67A/pDNA aerosols generated would be considered too large (MMAD>5 μm) for practical delivery to the lungs of patients.

Interestingly, the observed MMADs for GL67A/pDNA formulations were much higher than those reported in previous studies (ref). Eastman et at reported an MMAD of 2.2 μm for GL67A/pDNA formulations using the Pari LC+ nebulizer operating at 50 psi. In this study an MMAD of 4.3±0.4 μm (FIG. 3b) was observed using the same nebuliser under identical operating conditions. Although different plasmids were used in the two studies this would not be expected to impact droplet size significantly and it is likely that the observed difference is a result of improvements in aerosol sizing methodologies. The Andersen Cascade Impactor utilised for aerosol sizing in previous studies has been associated with undersizing of droplets caused by heating and evaporation during transit through the device. In contrast, assessment of aerosol characteristics in this study was carried out using a Next Generation pharmaceutical Impactor (NGI) operated under conditions optimised to prevent aerosol evaporation. Aerosol collection was performed using an airflow of only 15 L/min and using the NGI chilled to 4-7° C. to prevent evaporation of aqueous phase from the droplets. These optimised conditions have been accepted as a more realistic interpretation of true aerosol droplet size by the European Pharmaceutical Aerosol Group and represent current best practise in the field.

Whilst droplets <5 μm in diameter are regarded as broadly respirable, effective delivery of therapeutic formulations to target regions within the lung requires aerosols with appropriate MMADs. Excessively large droplets are rapidly filtered from inhaled air by deposition in the upper respiratory tract and conversely, small droplets can be exhaled without deposition. The desired target region of the lung will vary with each disease and although modelling of aerosol deposition in CF patients has proven challenging due to the presence of mucous plugs and inflammation, it is believed that aerosol with an MMAD of around 3 μm would be optimal for targeting the bronchial epithelium associated with CF lung disease. Due to the nature of GL67A/pDNA formulations, such aerosols proved difficult to achieve unless nebulisers were operated at relatively high pressures. Such pressures are high for medical nebulisers and would most likely preclude the use of portable compressor devices for clinical studies. Whilst easy to use and convenient for patients, portable compressors cannot generally achieve the required operating pressures and an alternative air source such as a compressed air cylinder would be required. Importantly, the required gas pressures also exceed the manufacturer's recommended operating conditions for many of the tested nebulisers (Table 3). As well as introducing safety concerns for patient operation, it would appear that the breath enhancement mechanism that increases the proportion of aerosol generated during patient inhalation in many of the tested nebulisers is impaired at higher pressures (FIG. 4d). Consequently, the efficiency of aerosol delivery to the patient lung is compromised—greatly reducing the appeal of these devices for GL67A/pDNA delivery.

In contrast to the other nebulisers examined, the breath actuated AeroEclipse II is certified to operate safely at 50 psi (Table 3) and is capable of efficiently delivering over 80% of generated aerosol to the patient (FIG. 4d). Breath actuation greatly reduces the amount of “wasted” aerosol produced during patient exhalation and simultaneously minimises environmental contamination and increases the proportion of aerosol delivered to the lung. As such it is a highly desirable property for any potential study incorporating expensive biological reagents such as gene therapy formulations. In previous studies utilizing GL67A/pDNA, breath actuation has been achieved by incorporation of a separate breath actuation module (Autoneb breath actuation device—Vortran Medical Technology) between the patient and the nebuliser. Unfortunately, this device is no longer clinically available and although increased delivery efficiency could in theory be achieved in conjunction with a simple home-made breath-actuation device, the availability of the disposable AeroEclipse II nebuliser greatly simplifies the clinical choice.

TABLE 2

| | Viacosity | Surface Tensions | | |
|---|---|---|---|---|
| Formulation | [symbol] | m [symbol] m | Particle Size nm | Zeta potential mV |
| Water | 1.00* | 72.8 ± 0.1 | n/a | n/a |
| 1% salbutamol | 1 ± 0.05 | 59.8 ± 0.4 | n/a | n/a |
| PEI/pDNA | 1 ± 0.06 | 72.5 ± 0.4 | 75 ± 4 | plus 47.3 ± 0.7 |
| GL67A/pDNA | 8.32 ± 0.09 | 42.2 ± 0.3 | 930 ± 130 | minus 19.5 ± 1.5 |

*reference [symbol]
indicates data missing or illegible when filed

TABLE 3

| [symbol] | [symbol] | Type | [symbol] | [symbol] | [symbol] | [symbol] | [symbol] | [symbol] |
|---|---|---|---|---|---|---|---|---|
| [symbol] | [symbol] | [symbol] | [symbol] | [symbol] | NA | YES | YES | NO |
| [symbol] | [symbol] | [symbol] | [symbol] | [symbol] | NA | YES | YES | NO |
| [symbol] | [symbol] | [symbol] | [symbol] | [symbol] | NA | YES | YES | NO |
| [symbol] | [symbol] | [symbol] | [symbol] | [symbol] | [symbol] | YES | YES | [symbol] |
| [symbol] | [symbol] | [symbol] | [symbol] | [symbol] | [symbol] | YES | YES | [symbol] |
| [symbol] | [symbol] | [symbol] | [symbol] | [symbol] | [symbol] | YES | YES | YES |
| [symbol] | [symbol] | [symbol] | [symbol] | [symbol] | [symbol] | YES | YES | YES |
| [symbol] | [symbol] | [symbol] | [symbol] | [symbol] | [symbol] | YES | YES | YES |
| [symbol] | [symbol] | [symbol] | [symbol] | [symbol] | [symbol] | YES | YES | YES |
| [symbol] | [symbol] | [symbol] | [symbol] | [symbol] | [symbol] | YES | YES | YES |
| | | | [symbol] | | | | | |

[symbol]

indicates data missing or illegible when filed

TABLE 4

| | [symbol] | | | [symbol] | | |
|---|---|---|---|---|---|---|
| | LC+ | [symbol] | [symbol] | LC+ | [symbol] | [symbol] |
| MMAD (μm) | 5.5 ± 0.1 | 4.4 ± 0.3 | 3.8 ± 0.1 | 4.3 ± 0.4 | 3.4 ± 0.1 | 3.4 ± 0.1 |
| FPF (% <5 μm) | 44.8 ± 1.5 | 58.3 ± 1.4 | 68.7 ± 1.5 | 30.9 ± 5.5 | 74.9 ± 1.4 | 71.4 ± 1.5 |
| Delivery rate ( [symbol] ) | 0.25 ± 0.01 | 0.15 ± 0.01 | 0.11 ± 0.01 | 0.38 ± 0.01 | 0.34 ± 0.01 | 0.17 ± 0.01 |
| Delivery efficiency (% total amount) | 53.5 ± 0.4 | 66.8 ± 1.1 | 53.7 ± 2.7 | 37.4 ± 0.4 | 55.3 ± 2.2 | 50.0 ± 2.3 |
| Dead volume (ml) | 0.64 ± 0.05 | 0.54 ± 0.11 | 0.48 ± 0.12 | 0.61 ± 0.06 | 0.74 ± 0.01 | 0.53 ± 0.10 |
| 10 ml delivery time ( [symbol] ) | 40 | 67 | 80 | 26 | 29 | 69 |
| Estimated lung delivery volume | 2.6 ml | 3.65 ml | 5.15 | 3.25 ml | 3.83 ml | 6.3 |
| (Res vol − dead VOL) × FPF × efficiency | | | | | | |
| Estimated lung delivery mg | 7.07 mg | 9.72 mg | 13.3 mg | 3.65 mg | 10.11 mg | 14.00 mg |
| (Res vol − dead vol) × FPF × efficiency × conc | | | | | | |

[symbol]

indicates data missing or illegible when filed

### EXAMPLE 4

### Data Concerning the Clinical Trials of the pGM69/GL67A Gene Therapy Product

### A. Single Dose Clinical Trial: Choice of the 5 ml Dose.

Patients were treated with nebulised 20 ml, 10 ml or 5 ml doses of the pGM169/GL67A formulation.

### Safety

There was a dose-related effect on temperature, systemic inflammatory markers and spirometry (FIGS. 8-11). Such effects may be described as flu-like symptoms.

The 20 ml dose gave statistically significant elevations in temperature compared to the 5 ml dose (FIG. 8); in addition, there were greater increases in peripheral white blood cells (FIG. 9) and C-reactive protein. With respect to lung function, the 20 ml dose elicited a significantly more pronounced and drop in FEV₁ than the 5 ml dose (FIG. 11). No statistically significant differences were observed between the 10 ml and 5 ml doses in any measure. However, in each of the key parameters measured, severity of outcome correlated strictly with dose, with the 5 ml dose having the least deleterious effects.

### Efficacy

In 10 patients treated with 20 ml nebulised doses, bronchial potential difference (bPD) measurements demonstrated a significant change towards normal values. Nasal dose was standard at 2 ml (after the 1^{st} 3 patients had safely received 1 ml). Evidence for a positive treatment effect was seen in approximately half the patients on nasal PD. Of particular note, long term correction of nPD was seen in some patients (e.g. see FIG. 12). Transgene mRNA in both nasal and bronchial samples was quantified by RT-PCR; a subgroup of patients were positive although this did not correlate with functional correction.

In patients treated with 20 ml, 10 ml and 5 ml doses, lung function was evaluated pre- and post-treatment by measurement of lung clearance index (LCI). Statistically significant evidence of a positive treatment effect was seen in the 10 ml and 5 ml groups, but not in the 20 ml group (FIG. 13).

### Inflammation/Efficacy Interaction

FIG. 14 informally tabulates inflammation and efficacy measures. There was no obvious correlation between the severity of the inflammatory responses observed and the molecular efficacy recorded. We conclude that efficacy is not tightly coupled to inflammation.

### Conclusion

5 ml (containing 13.25 mg pGM169 DNA) is a well tolerated dose, which does not produce flu-like symptoms or significant pulmonary reactions, and is suitable for use in the multi-dose trial. Higher doses (10 and 20 ml) produce overt symptoms which are clearly undesirable and would also likely unblind the trial. Interestingly paracetamol (shown in FIGS. 8-11 as orange symbols) given over the first 24 hours is able largely to abrogate these changes despite neither ibuprofen nor systemic corticosteroids appearing effective (data not shown).

### B. Pre-Clinical Data to Support Multi-Dose Trial Design

### Toxicology Studies

In preparation for a Multi-dose clinical trial to assess whether 12 monthly doses of the CFTR gene can improve CF lung disease we have conducted two toxicology studies (TS). In addition to a GLP-mouse TS study (outsourced to a CRO) we also performed an in-house study in sheep. Mice received 12 doses of pGM169 (a plasmid carrying the CFTR gene) complexed to Genzyme lipid mixture GL67A at fortnightly intervals by inhalation; exposure to the complex was for 0.5 (low=˜5× human dose), 2 (mid=˜20× human dose) or 6 hr (high dose=˜60× human dose). Controls were exposed to air for 6 hr. In addition to standard non-invasive assessments at regular intervals and post-mortem toxicology undertaken 2 weeks and 3 months after the last dose, molecular analyses were performed to assess gene expression and biodistribution (see summary below). Approximately 600 mice were included in the study. Anesthetised sheep (n=8) received 9 doses (consistent with EMEA guidelines) of aerosolized pGM169/GL67A at monthly intervals. Similar assessments were made as for the mouse TS, in addition to lung function measurements and bronchoalveolar lavage (BAL) at intervals throughout the study. The amount (mg/kg) of pGM169/GL67A delivered to sheep approximately mimics the proposed human dose. Anesthetised controls (n=8) received air only. Results: All mice tolerated the treatment well. In the high dose group (˜60× human dose) small increases in lung weight and circulating neutrophils were seen 2 days after the last dose, but not in the cohort sacrificed 14 days after the last dose. Histology showed scattered alveolar macrophages in the low and mid dose groups 2 weeks after administration of the last dose. In the high dose group multifocal alveolar foamy macrophage accumulations and occasional inflammation were noted. The presence of fat in macrophages was confirmed by Oil Red 0 staining. There was no evidence of structural remodelling. All other organs were unremarkable. 3 months after administration, findings in the low dose were not different from baseline, but were still observed in the mid and high dose. All sheep tolerated treatment well. In contrast to mice, haematology 2 or 14 days after dosing did not show any treatment related effects. Neutrophils were transiently increased in BAL fluid 2 days after administration, but had returned to baseline after 15 days. Observed changes in lung function or gas transfer measurements were mild and not test item related. There were no test item related histological findings in any organ. Conclusions: All animals tolerated the treatment well. The transient and dose-related systemic inflammatory responses and drop in lung function observed in the single-dose phase 1 trial (separate abstract) were not replicated in non-CF sheep and non-CF mice only developed mild systemic inflammation at the highest dose (˜50× human dose) possibly highlighting species-specific differences or an increased response to the lipid/DNA complexes in the inflamed human CF lung. Both TS support progression into a Multi-dose CF gene therapy trial.

### Gene Expression and Biodistribution Studies

The nebulisation of multiple doses of a non-viral gene therapy formulation to the lungs of individuals with CF is being considered as a treatment for chronic CF lung disease. The success of such a strategy depends on the selection of a formulation capable of CFTR expression that is high-level, sustained, and can be repeatedly administered. Plasmid pGM169 is free of CG dinucleotides and contains the novel synthetic hCEFI promoter specifically designed for long-term expression of CFTR in the lung; when complexed with cationic liposome GL67A (supplied by Genzyme) CFTR expression lasts for several months in the mouse lung after a single nebulised dose. In order to progress into multiple-dose studies a GLP toxicology (see above) and biodistribution study was performed in mice to evaluate the CFTR expression profile of up to 12 doses of pGM169/GL67A. Mice were exposed to aerosolised pGM169/GL67A at 2 weekly intervals for 0.5, 2 or 6 hours (Low, Medium and High dose groups respectively); mice exposed to air for 6 hours were used as controls. Groups of mice (males & females; n=4-10) were sacrificed after one, six and 12 doses and organs (lung, cervical lymphnodes, spleen, gut (proximal ileum), liver, kidney, testes/ovaries, and blood) analysed for the presence of plasmid DNA and/or vector-specific CFTR mRNA using quantitative RT-PCR.

DNA deposition was dose-dependent. A significant dose-response was observed between duration of inhalation and the quantity of plasmid DNA present in the lungs 1 day after delivery of one, six and 12 doses (p<0.0001; Spearman correlation). Plasmid DNA remained detectable in the lungs of animals for up to 21 weeks after the final (High) dose. Levels of plasmid DNA in non-target organs were several orders of magnitude lower than the lungs at day 1. When CFTR mRNA was measured in the lungs, low levels were detected after a single dose in the Low and Medium groups, with increased signal in the High group (p<0.001; equivalent to ! 100% endogenous levels). Importantly, after 12 doses, a cumulative treatment effect was noted such that high mRNA levels were observed for all animals in all treatment groups. Robust levels of CFTR mRNA remained in the lung for at least 21 weeks after the final exposure.

### SUMMARY

This is the first time that multiple nebulised doses of a lipid-based formulation have been shown to achieve a long-lasting and cumulative CFTR expression that can build with successive doses; importantly this confirms our clinical strategy to deliver multiple doses in order to maximise CFTR expression. In conclusion, these studies further support progression into our planned Multi-dose clinical trial designed to generate clinical benefit in CF patients.

### EXAMPLE 5

### Dose Preparation and Administration Protocol for pGM169/GL67A

### Summary

Set out below are details of the preparation and administration of the nasal and lung dose of pGM169/GL67A. This includes assembly of the nebuliser, filling, weighing and administration of the nebulised lung dose, and assembly and administration of the nasal dose. In part A, patients receive a nasal and lung dose of pGM169/GL67A. Patients in part B may receive either a lung dose, nasal dose or both. This will be fully documented in patient notes.

### Equipment

### Nasal Spray and Dose

Nasal spray bottle (GSK part No. AR5989 30ML) (B in picture below)
Nasal spray actuator (GSK Part No. AR9488 30ML) (A in picture below)
10 ml GL67/pGM169 complexes prepared as described in the Example above.

### Nebuliser

Each 10 ml lung dose patient requires 2× AeroEclipse II BAN kits.
Each 20 ml lung dose patient required 4× AeroEclipse II BAN kits.

The AeroEclipse II BAN kit contains: Instruction manual; Nebuliser tubing with hose connectors; AeroEclipse II BAN; Standard white mouthpiece (not used).
Piped medical air point in dosing cubicle
Air Regulator showing flow rate (8 l/minute)
Nebuliser pot stand (holds nebuliser upright when not in use)
Tape to hold down nebuliser pot stand
Scales—Mettler AJ100
Pari Filter Set. Contains: Instruction manual; Inspiration valve for LC nebulisers (not used); Exhalation filter unit (PARI Part No. 41B0519) pre-fitted with a single filter pad (PARI Part No. 41E0522); Mouthpiece (without valve—this is the mouthpiece that is required for dosing) (PARI Part No. 12E1720); Y Piece adapter (PARI Part No. 41E0530).

### Procedures

### Procedure to Fill Nasal Bottle

Clean worktop with Clinell surface wipes.
Place sterile drape over work surface
Check details on label to confirm expiry time according to pharmacy label and complete Dose Request Master Form.
Transfer all 10 ml of GL67/pGM169 complexes (prepared as described in the examples herein) from Sterilin tube into nasal spray bottle using a sterile 10 ml syringe (no needle attached).
Insert nasal spray actuator into nasal spray bottle.
Screw actuator into position by pushing down and twisting clockwise until locked.
Once locked into position, device cannot easily be re-opened.
Remove protective cap from nasal spray device.
Prime by placing into adapted falcon tube. Holding spray upright activate 20 times
Nasal spray device is now ready to use.
This spray bottle will be used to deliver either 1 ml or 2 ml of dose as prescribed (i.e. the same 10 ml aliquot is used for both nasal doses).
Weigh nasal bottle post priming with lid (pre use with patient). NB The bottle is weighed again at the end—see below.

### Filling of Nebuliser Pot

Keep same drape as for nasal spray bottle.
1. Clean work surface with hard surface wipe
2. Place clean drape (can use the same drape if also having nasal dose at same time.)
3. Open AeroEclipse II BAN nebuliser pack.
4. Remove mouthpiece from nebuliser aperture and discard this mouthpiece.
5. Attach straight connector (Product no: 812-106) to air inlet on nebulizer pot
6. Place nebuliser pot in 60 ml Sterilin
7. Label nebuliser pot and sterilin A-D as follows: for first 5 ml dose label both nebuliser pot and sterilin ‘A’, and second 5 ml dose label ‘B’ etc.
8. Remove lid from AeroEclipse II nebuliser by gently twisting the lid anticlockwise and lifting out of nebuliser body.
9. Transfer 5 ml GL67/pGM169 directly into nebuliser reservoir using a 10 ml syringe. Note, if the patient is prescribed only 2.5 ml then transfer only this amount into the nebuliser reservoir.
10. Retain remainder of GL67/pGM169 formulation, ensuring lid in place. Store out of direct sunlight.
11. Replace lid onto nebuliser. Ensure lid is securely in place by rotating lid clockwise until locked into position.
12. Weigh pot as described below (make sure nebuliser and sterilin have matching letters (e.g. both are ‘A’ etc)

Each new aliquot of 5 ml will repeat steps above with new nebuliser kit and sterilin. 5 ml aliquots will be used where possible. If the remaining volume to be delivered is <5 mls, then this amount will be added using 10 ml syringe to measure volume. E.g. If 7.5 ml total dose is to be given, transfer 5 mls into the nebuliser pot A and 2.5 mls into pot B etc.

### Weighing Nebuliser Pot Pre-Dosing

Zero weighing scales and take nebuliser pot filled with 5 ml dose (or 2.5 ml if lower volumes prescribed) of pGM169/GL67A as described above. It should be weighed without mouth piece and filter.
Place sterilin with nebuliser pot onto scales.
Record weight on the administration work sheet rounding up or down to nearest 0.1 g (example: weight=41.03 g, record as 41.0 g; weight=41.08 g, record as 41.1 g) on Administration worksheet according to whether patient is receiving 10 or 20 mls. Pots are referred as A-D on this sheet for each 5 ml (or 2.5 ml) dose.

### Procedure for Lung (Nebulised) Dosing

Attach filter unit and mouthpiece from filter set (no valve). Note filter unit tilts away from patient)
Attach tubing to nebuliser as in picture 2 and secure in place. Between Air inlet and tubing will be attached straight connector as mentioned above. In the tubing push in a hose barb connector (No: 227-566).
Attach other end of tubing to air outlet.
Ensure AeroEclipse II nebuliser is in breath actuation mode by rotating the mode selector on the lid until arrow points to breath actuation symbol
Teach patient how to turn to ‘continuous’ mode (900 turn to solid triangle), which they will need to do later in procedure
Ensure valves are functioning correctly by asking patient to take several breaths through the nebuliser before the compressed air supply is turned on. Also check that blue flap is moving up on exhalation in filter exhalation vent
Ensure that regulator is attached correctly to wall air point as trained
Remind the patient to report immediately any symptoms to nurse/doctor with them at dosing.
Turn on air until regulator reads 8 litres/min (line middle of ball if standard flow regulator with ball float or turn regulator to 8 litres/min if direct dial)
Staff member can leave cubicle and close door. Apron, mask and gloves should be deposited in clinical waste bags (orange bags). Hands should be cleaned with alcohol rub
Ask patient to place mouthpiece in mouth, form a seal with lips and breathe normally; avoid breathing through nose.
Ensure feedback button on lid of nebuliser falls during inhalation and rises during exhalation; this confirms mouth (rather than nose) breathing.

### Procedure for Nasal Dosing

Nasal spray to be given in the rest period after each period of nebulisation (e.g. after each 3 mins of nebulisation on standard protocol, or 1 minute on slow delivery rate protocol).
Patients having just nasal dose dosing to occur every 5 mins
One actuation/rest period to both nostrils. Record each actuation on worksheet and keep in CRF at end of the study visit.
A patient prescribed a 1 ml nasal dose of pGM169/GL67A would receive a total of 12 nasal spray actuations (6 to each nostril).
A patient prescribed a 2 ml nasal dose of pGM169/GL67A would receive a total of 24 nasal spray actuations (12 to each nostril).
Complete worksheet for giving nasal administration of pGM169/GL67A

### Protocols for Dosing

### For Patients in Standard Delivery Rate Protocol

1. Patient to breathe on nebuliser for around 3 minutes. Nurse will indicate to patient when this time has elapsed.
2. Nebuliser mouthpiece removed from mouth and pot placed in holder in cubicle. Do not detach or switch off the air supply.
3. Approximate 2 minute break from nebulisation, during which nasal dose will be administered by patient if having nasal dose.
4. The nasal spray is to be recorded on the worksheet each time an actuation is given up to a total of 6 into each nostril for 1 ml dose or 12 into each nostril for 2 mls.
5. Every 15 minutes or sooner if mist is seen coming out of filter top during expiration filter should be changed. This is done as described below. Staff should wear PPE whilst doing this.
6. Every 30 mins a staff member will enter cubicle in (PPE) and measure oxygen saturation and heart rate.
7. Stages 1-3 will be repeated for a total of 8 cycles (total approximately 40 minutes).
8. Staff member then asks patient to turn mode selector on top of nebuliser to ‘continuous’
9. If a jet is visible from mouthpiece, patient is asked to repeat stages 1-3 (without nasal dosing) twice more or longer until no jet is visible. If this is only than a further 30 minutes, further saturation and heart rate observations will be made.
10. When no jet is visible from mouthpiece, the nebulisation is considered complete; NB there will still be fluid visible in the nebuliser chamber
11. Staff member to wear PPE, enter cubicle, turn off wall air source and remove nebuliser pot for weighing as described earlier
12. Patients will have a break of approximately 10 minutes before repeating this process for further nebuliser pots as prescribed.
13. They may remain in the cubicle or if they wish to leave they will wear a face mask.

### For Patients in Slow Rate Delivery Protocol

1. Patient to breathe on nebuliser for 1 minute and rest for 4 mins. Nurse will indicate to patient when this time has elapsed
2. Nebuliser mouthpiece removed from mouth and pot placed in holder in cubicle. Do not detach. Turn off air supply when doing slow delivery otherwise leave running for standard delivery.
3. Change filter if mist is seen coming out of filter top during expiration. This is done as described below. Staff should wear PPE whilst doing this.
4. Every 30 mins a staff member will enter cubicle in (PPE) and measure oxygen saturation and heart rate.
5. Stages 1-5 will be repeated until nebuliser post is completed (total approximately 75 mins (slow)
6. Staff member then asks patient to turn mode selector on top of nebuliser to ‘continuous’
7. If a jet is visible from mouthpiece, patient is asked to continue regime twice more or longer until no jet is visible. If this is only than a further 30 minutes, further saturation and heart rate observations will be made.
8. When no jet is visible from mouthpiece, the nebulisation is considered complete; NB may be a small amount of fluid visible in the nebuliser chamber
9. Staff member to wear PPE, enter cubicle, turn off wall air source and remove nebuliser pot for weighing as described earlier
10. Patients will have a break of approximately 10 minutes before repeating this process for further nebuliser pots as prescribed (there will not be any further pots if patient only to receive 5 ml or 2.5 ml total lung dose).
11. They may remain in the cubicle or if they wish to leave, they will wear a facemask if 30 mins post nebulisation has not elapsed.

### Changing Nebuliser Filters

Filters should be changed at intervals as described herein
Turn off compressed air source and remove AeroEclipse II nebuliser and exhalation filter unit from patient.
Remove filter unit and stand nebuliser pot in stand on worktop in dosing room.
Open filter unit by rotating filter unit lid anti-clockwise and lifting to reveal filter pad.
Remove used filter pad and place in plastic box (with lid) on the worktop in dosing cubicle
Replace filter pad and close unit turning lid clockwise
Replace filter unit and mouthpiece to nebuliser pot.

### Weighing Nebuliser Pot Post-Dosing

A Repeat weighing as for pre-dosing except the nebuliser pot will now have no or little product left in it.
B. Follow worksheet to calculate weight of pGM169/GL67A dose at the end of dosing.

Repeat all of the weighing for each aliquot (5 mls or less as described) being nebulised.

NB Each aliquot will require a new nebuliser pot, filter and mouthpiece. The same tubing is used for all nebuliser pots for 1 patient. The nebuliser pot is weighed pre and post every aliquot and weight recorded on the worksheet.

### When Lung and or Nasal Dosing Completed (Including Nebulisers Weighed)

Place all nebulisers with mouthpiece and filter in the bucket with Trigene in and leave for 2 hrs then throw away in orange bins. (See Waste Removal SOP 620)
Nasal spray bottles:
   weigh the bottle at the end of dosing (remember to do this before emptying described below).
   Place the tip of the nasal spray bottle into the adapted Falcon tube as done for initial priming. Empty all the remaining contents of the nasal spray bottle into the Falcon tube taking care not to spill the gene therapy product. (NB: It may be necessary to use more than one Falcon tube as the tissue inside may become saturated). Then place both nasal spray bottle and adapted falcon tube(s) into the Trigene and leave for at least 2 hrs then throw away in orange bin as above.
Also putting into Trigene for 2 hrs applies to all other used filters and filter box.

### EXAMPLE 6

### Multiple Doses of Lipid-Mediated Gene Therapy Nebulised to the Mouse Lung Show Robust and Sustained CFTR Expression

The nebulisation of multiple doses of a non-viral gene therapy formulation to the lungs of individuals with CF is being considered as a treatment for chronic CF lung disease. The success of such a strategy depends on the selection of a formulation capable of CFTR expression that is high-level, sustained, and can be repeatedly administered. Plasmid pGM169 is free of CG dinucleotides and contains the novel synthetic hCEFI promoter specifically designed for long-term expression of CFTR in the lung; when complexed with cationic liposome GL67A CFTR expression lasts for several months in the mouse lung after a single nebulised dose. A clinical trial to assess the safety and CFTR expression of a single nebulised dose of pGM169/GL67A in 35 CF patients has just been completed. In order to progress into multiple-dose studies a GLP toxicology and biodistribution study was performed in mice to evaluate the CFTR expression profile of up to 12 doses of pGM169/GL67A. Mice were exposed to aerosolised pGM169/GL67A at 2 weekly intervals for 0.5, 2 or 6 hours (Low, Medium and High dose groups respectively); mice exposed to air for 6 hours were used as controls. Groups of mice (males & females; n=4-10) were sacrificed after one, six and 12 doses and organs (lung, cervical lymphnodes, spleen, gut (proximal ileum), liver, kidney, testes/ovaries, and blood) analysed for the presence of plasmid DNA and/or vector-specific CFTR mRNA using quantitative RT-PCR. A significant dose-response was observed between duration of inhalation and the quantity of plasmid DNA present in the lungs 1 day after delivery of one, six and 12 doses (p<0.0001; Spearman correlation). Plasmid DNA remained detectable in the lungs of animals for up to 21 weeks after the final (High) dose. Levels of plasmid DNA in non-target organs were several orders of magnitude lower than the lungs at day 1. When CFTR mRNA was measured in the lungs, low levels were detected after a single dose in the Low and Medium groups, with increased signal in the High group (p<0.001; equivalent to !100% endogenous levels).

Importantly, after 12 doses, a cumulative treatment effect was noted such that high mRNA levels were observed for all animals in all treatment groups. Robust levels of CFTR mRNA remained in the lung for at least 21 weeks after the final exposure. This is the first time that multiple nebulised doses of a lipid-based formulation have been shown to achieve a long-lasting and cumulative CFTR expression that can build with successive doses; importantly this confirms our clinical strategy to deliver multiple doses in order to maximise CFTR expression. In conclusion, these studies further support progression into our planned Multi-dose clinical trial designed to generate clinical benefit in CF patients.

### EXAMPLE 7

### Safety and Expression of a Single Dose of Lipid-Mediated cftr Gene Therapy to the Upper and Lower Airways of Patients with cf

In preparation for a Multi-dose clinical trial (MDT) of CFTR gene therapy, we undertook a study to assess safety and dose-ranging with a single nebulised dose of pGM169/GL67A [a CpG-free human CFTR plasmid with a CpG-free CMV enhancer and human elongation factor 1 alpha (hCEFI) promoter complexed with Genzyme Lipid 67A. We also assessed transgene expression in the lung and upper airway with QRT-PCR on airway brushings and nasal and bronchial potential difference measurements.

35 adult patients with a baseline FEV₁ of at least 60% predicted received a nebulised dose of 20 ml (n=17), 10 ml (n=10) or 5 ml (n=8). A short-lived, dose-related drop in FEV₁ was observed over the next 6 hours (mean [SD]: 20 ml 25.7[10.2]%; 10 ml 17.7[9.9]%; 5 ml 13.0[4.4]%); this was well-tolerated, not accompanied by desaturation, showed a restrictive pattern and was unresponsive to bronchodilators. Subjects also experienced a systemic inflammatory response, the symptoms of which responded well to anti-pyretics. This was similarly dose-related and limited, in general, to the first 24-48 hours post-dosing. A significant correlation between the magnitude of the FEV₁ drop and the rise in white blood count, suggests that the former may be an inflammatory phenomenon. A cohort of 6 patients (4@10 ml; 2@5 ml) received 4 g paracetamol over an 18 hour period post-dosing; none of these patients developed a fever. Intriguingly, these subjects also appeared to have reduced systemic inflammatory responses.

Ten patients underwent pre and post-dosing bronchoscopies, the latter at either day 6 or day 14 post-dose. Molecular (mRNA) evidence of gene transfer was observed in some individuals. The majority of the patients' zero chloride isoproterenol responses increased towards non-CF values after gene therapy. 19 patients received a 2 ml nasal dose. Based on an a priori definition of a ‘positive response’ as a chloride secretory response outside the range of their personal pre-dose measurements, 11/19 patients responded. The plasmid has been engineered to produce slow onset of transgene expression, but with extended duration. Interestingly, PD was notably more frequently positive when measured at later, rather than earlier time points. In the two most positive individuals, maximal zero chloride responses exceeded 10 mV (ie. in the normal non-CF range) and persisted to days 63 and 91 respectively.

In conclusion, we consider the pulmonary and systemic side effects after 20 ml nebulised dose are excessive for repeated application. Those at 10 ml were more acceptable, whilst 5 ml produced only small changes. Symptoms are amenable to simple antipyretic treatment and the MDT will use either 5 or 10 ml. Gene expression was confirmed in both the lower and upper airways, and restoration of CFTR function in to the non-CF range has been observed out to 13 weeks following a single dose to the nose. These data, together with our preclinical toxicity packages support progression of this agent to the Multi-dose clinical trial.

### EXAMPLE 8

### Safety and Expression of a Single Dose of Lipid-Mediated CFTR Gene Therapy to the Upper and Lower Airways of Patients with Cystic Fibrosis

### Introduction and Objectives

We undertook a clinical trial of non-viral CFTR gene therapy assessing safety, dose and transgene expression in preparation for a Multi-dose trial (MDT) designed to assess clinical efficacy.

### Methods

A single nebulised and/or nasal dose of plasmid CFTR (pGM169)/GL67A was delivered to patients aged ≧16 years with a baseline FEV₁>60% predicted. Clinical and laboratory parameters were measured at intervals until day 28. A cohort of patients also underwent pre and post-dosing (day 6 or 14) bronchoscopies for functional (airway potential difference (PD)) and molecular (QRT-PCR) evidence of vector-specific CFTR expression. Patients receiving a nasal dose underwent brushings for QRT-PCR and serial nasal PD measurements.

### Results

35 patients received a nebulised dose of 20 ml (n=17), 10 ml (n=10) or 5 ml (n=8). A short-lived, dose-related drop in FEV₁ was observed over the next 6 hours (mean[SD]: 20 ml 25.7[10.2]%; 10 ml 17.7[9.9]%; 5 ml 13.0[4.4]% of baseline). Subjects also experienced a systemic inflammatory response which was similarly dose-related and generally limited to the first 24-48 hours post-dosing. A cohort of 6 patients (4@10 ml; 2@5 ml) received 4 g paracetamol over an 18 hour period post-dosing; none of these patients developed a fever. Intriguingly, these subjects also appeared to have reduced systemic inflammatory responses.

Molecular (mRNA) evidence of gene transfer was observed in some individuals from upper or lower airway brushings. On lower airway PD measurement, the majority of patients showed an increase towards non-CF values after nebulised gene therapy.

19 patients received a 2 ml nasal dose and 11 (58%) had some response in chloride secretion on nasal PD. In the two most positive individuals, responses were within the normal (non-CF) range and persisted to days 63 and 91, respectively.

### Conclusions

We consider the side effects after 20 ml nebulised dose excessive for repeated application. Those at 10 and 5 ml were more acceptable. Gene expression was confirmed in some patients, and restoration of CFTR function to the non-CF range has been observed out to 13 weeks following a single nasal dose. These data support progression of this agent to MDT.

### EXAMPLE 9

### Determining a Set of Biomarkers that can be Used to Monitor the Success of Cystic Fibrosis Treatment

We have taken a novel approach to recruiting patients into the Multidose trial, based around a ‘can deliver can measure’ strategy.

The CF airways gradually fill with mucus making delivery of the gene therapy increasingly difficult. It is therefore likely that the milder the disease severity the higher the chance of success (‘can deliver’). All our patients in the Run-in inhaled a radiolabelled aerosol to define the patency of their airways.

However, the milder the disease severity, the lower the likelihood that biomarkers of disease severity will be abnormal. Thus whilst it may be easier to deliver the gene therapy, there may be no good way of measuring success. All our patients in the Run-in Study had a large panel of biomarkers measured over a two year period to ensure there would be suitable biomarkers (‘can measure’).

We then assessed all patients, removing ‘can't deliver’ or can't measure’, thereby maximising our chances of success in the trial ie a stratifed medicine approach to enrollment.

The longitudinal measurement of biomarkers for an extensive period pre-enrollment is very unusual for a Phase 2b study, and this combined with measurement of molecular biomarkers pre-treatment, will retrospectively give us a good chance of being able to identify the characteristics of responders and non-responders once the Multidose trial is completed. If appropriate a follow-on registration trial can then be enriched for potential responders ie a stratified medicine approach to biomakers.

During the Run-in and Tracking studies we identified the same four key biomarkers (FEV1, Lung Clearance Index, CT scan and Quality of Life questionnaire) which both distinguish CF from non-CF patients, and have low enough variability that they are of considerable potential as the key biomarkers for CF trials (whether gene therapy or small molecules).

### EXAMPLE 10

### The Importance of Appropriate Reference Equations for Spirometry: Lessons Learned from the Cystic Fibrosis Gene Therapy ‘Run-in’ Study

### Introduction and Objectives

The ‘Run-In’ study is an ongoing longitudinal, observational study of CF patients assessing outcome measures for a future gene therapy trial.

Spirometry is performed at each visit and volumes are converted to % predicted values according to published reference equations; historically these were separate for adults and children. Here, we describe the issues arising from this approach, and highlight the benefit of using a reference source which bridges the transition from child to adulthood.

### Methods

CF subjects (≧10 years; FEV₁≧40% predicted) were recruited from three sites in London and Edinburgh. Visits were undertaken during periods of stability every 3-6 months; data presented here are from the first 4 visits. Spirometry was performed on an Easyone spirometer. Volumes were converted to % predicted values according to Rosenthal (<18 years) and Quanjer (≧18 years) reference equations. The FEV₁ raw data were subsequently re-analysed using Stanojevic reference equations, which span all age ranges. Comparisons were made using paired t-tests.

### Results

191 patients attended visit 1 (mean age 22.7 years, 55% male; 91 patients <18 years). Rosenthal and Quanjer FEV1% predicted values were significantly higher than the Stanojevic values: mean differences 2.8 (95% CI 1.9-3.7) for children with Rosenthal equations (p<0.0001), and 2.4 (95% CI 2.1-2.8) for adults using Quanjer equations (p<0.0001).

10 patients transitioned between paediatric and adult reference ranges during the study period; the slope of change in their FEV1% over visits 1-4 was significantly greater with Rosenthal/Quanjer references than with Stanojevic (p=0.001) largely due to an artefactual drop when switching from Rosenthal to Quanjer values. As an example, a female patient aged 17.8 years at visit 1 had a drop in absolute FEV₁% predicted between visits 1 and 2 of 11% when Rosenthal/Quanjer were used but only 3% with Stanojevic reference values.

### Conclusions

Our results highlight issues raised when separate adult and paediatric spirometry reference ranges are used in longitudinal study. The UK CF Gene Therapy Consortium has adopted the Stanojevic reference source for all spirometry analysis in its ongoing Clinical Programme.

### EXAMPLE 11

### Deposition Scan

### Purpose

To determine which patients would be most likely to be optimal for topical drug delivery by assessing deposition of a radiolabelled aerosol.

### Technique

^{99m}Tc-labelled human serum albumin was inhaled via a Pari LC nebuliser & Akita system to enhance conducting airway deposition. Anterior and posterior planar gamma camera images and SPECT were used to assess 3-D deposition. Images were scored digitally and, after coding, they were visually graded I-IV by a group of respiratory physicians as follows: I—no defects; II—patchy deposition; III—patchy deposition with large defects; IV-grossly abnormal (see Fig).

### Outcomes

Deposition scan were available on 147 subjects; digital indices ranged from 34 (best) to 150 (severely abnormal). Visual grading was well correlated with digital scores (R² 0.63; p<0.001) and both were significantly negatively correlated with lung function measured by FEV₁% (p<0.01). 9 subjects in Grade IV had a mean (SD) FEV₁ over all study visits of 43.9 (4.3) %, which was significantly lower than the means of groups I-III (p<0.01). They were also the most severely affected group for every parameter on chest CT scan.

### Conclusions

On the basis of these deposition scan appearances, Group IV was considered unsuitable to progress to the gene therapy trial. Patients in the other groups had deposition scans which suggested that the gene therapy product could be delivered at least moderately well to their airways and they were next filtered through other inclusion/exclusion criteria.

### EXAMPLE 12

### Change in Physiological, Functional and Structural Assessments of CF Lung Function with Treatment of CF Exacerbation

We aim to develop pulmonary gene therapy to patients with cystic fibrosis. In preparation for a multidose clinical trial and in parallel with the development of gene therapy products, we wished to determine the optimal clinical assays to gauge the effectiveness of such therapy and upon which primary and secondary outcome measures could be based. CF lung disease is characterised by recurrent ‘exacerbations’ manifest by increased symptoms and signs and from which up to 25% of patients fail fully to regain their baseline lung function. Such exacerbations are therefore crucially important in the natural history of the condition, which leads ultimately to respiratory failure and early mortality[1]. Disrupting and delaying this cycle of infection and impairment is a key principle of CF clinical management and the endeavour of much research for this disease.

We considered the treatment of a pulmonary exacerbation of CF lung disease with intravenous (IV) antibiotics to represent the most effective short-term therapy for CF pulmonary disease and one around which we could assess outcome measures. There have been a number of previous studies assessing the effectiveness of IV antibiotics on clinical assays in CF lung disease, demonstrating improvement in pulmonary function, inflammatory markers, radiology, and symptoms. Studies have generally included relatively small numbers of patients and a limited number of assays; results have been variable. No study to date has comprehensively provided a co-ordinated assessment of the key domains of CF lung disease: symptoms, function, structure, and inflammation.

Our hypotheses were a) that assays suitable as outcome measures a clinical trial would change with IV antibiotics for a pulmonary exacerbation, b) that studying them in a co-ordinated fashion would allow us to understand the relationships between different domains, broadening our understanding of a pulmonary exacerbation and c) that this would enable us to chose potential outcome measures for our future gene therapy trial. We also reasoned that such a study, co-ordinated across three sites, would lay the foundations and provide the practice necessary for the future success of the collaborative programme.

### Methods

Participants were requested to complete a series of assessments (Table 5) in a structured order at two time points: (visit 1) within 72 hours of commencing IV antibiotics for a pulmonary exacerbation (visit 2) within 5 days of completion of antibiotic therapy. In addition, patients were provided with a pocket electronic spirometer (Piko-1, Ferraris

Respiratory, Hertford, UK) with which to record a daily FEV₁ at home.

TABLE 5
Summary of assays performed at start and end of exacerbation in
order of sequence performed.

| Domain | Assay |
|---|---|
| Symptoms | Symptom score |
| Clinical Observations | Pulse |
| | Respiratory rate |
| | SpO₂ |
| | Temperature |
| | Blood pressure |
| | Weight |
| Lung Physiology | Spirometry |
| | Lung Clearance Index |
| Inflammation—Pulmonary | Sputum 24 hr dry weight |
| | Total & differential sputum cell count |
| | Sputum IL6, IL8, IL12, MMP9 |
| Inflammation—Systemic | Serum white cell count |
| | CRP |
| | Serum IL6, IL8, IL10, TNFα, |
| | Calprotectin |
| Structural* | CT score |

### Clinical Assays

### Symptom Score

A self-graded symptom score was developed and applied. Patients scored each of seven symptom related questions on a five point scale from −2 (much worse than normal) to +2 (much better than normal), with zero representing no change from normal for them. The final symptom score, obtained by summing the individual question scores, thus ranges from −14 to +14. Likewise, a negative change in symptom score after treatment represents a deterioration in symptoms, a positive score represents improvement and a zero score would be consistent with no overall change in patient-reported symptoms.

### Lung Clearance Index

Multiple breath washout was performed as previously described, using a modified Innocor gas analyser and 0.2% sulphur hexafluoride (SF6) as the tracer gas (Horsley, A. R., et al., Lung clearance index is a sensitive, repeatable and practical measure of airways disease in adults with cystic fibrosis. Thorax, 2008. 63(2): p. 135-40). LCI is quoted as the mean of at least two reproducible repeats from washouts of satisfactory quality. As an additional quality control measure, washouts whose FRC differed by more than 10% from both of the other two repeats were excluded from analysis.

### Spirometry

Baseline spirometry was measured according to American Thoracic Society/European Respiratory Society guidelines (Miller, M. R., et al., Standardisation of spirometry. Eur Respir J, 2005. 26(2): p. 319-38) using an Easyone spirometer (ndd Medizintechnik AG, Zurich, Switzerland). FEV₁ and mid-expiratory flows are expressed as standard deviation scores (SDS), or z scores, using reference ranges derived from the modified NHANES III database, as described by Stanojevic et al. (Stanojevic, S., et al., Reference ranges for spirometry across all ages: a new approach. Am J Respir Crit. Care Med, 2008. 177(3): p. 253-60). FEV₁ is also expressed as percent predicted using reference ranges provided by the European Community for Coal and Steel (adults≧17 years) (Quanjer, P. H., et al., Lung volumes and forced ventilatory flows. Report Working Party Standardization of Lung Function Tests, European Community for Steel and Coal. Official Statement of the European Respiratory Society. Eur Respir J Suppl, 1993. 16: p. 5-40) and Rosenthal et al. (children≦16 years) (Rosenthal, M., et al., Lung function in white children aged 4 to 19 years: I—Spirometry. Thorax, 1993. 48(8): p. 794-802). In n=x subjects Easyone spirometry was not obtained at Visit 2 in error. An analysis of PIKO daily handheld FEV₁ confirmed reliability of data compared to Easyone FEV₁ (online supplement) and PIKO derived FEV₁ for both Visits 1 and 2 was used for assessment of change in these patients.

### Sputum Preparation

Sputum was expectorated spontaneously (visit 1100% of patients; visit 2 85%) or induced using a standard methodology (Pavord, I. D., et al. The use of induced sputum to investigate airway inflammation. Thorax, 1997. 52(6): p. 498-501), modified as previously described (Gray, R. D., et al., Sputum proteomics in inflammatory and suppurative respiratory diseases. Am J Respir Crit. Care Med, 2008. 178(5): p. 444-52). Success rate of sputum induction useful here In brief, sputum plugs were harvested and processed with 4× weight/volume 0.1% dithiothreitol (DTT) after which 4× weight/volume PBS was added. Samples were filtered through 48 μm mesh and centrifuged at 1200 rpm to remove cells. Supernatant was stored at −80° C. until further analysis.

Total cell counts were obtained by counting cells in an improved Neubauer counting chamber. For differential cell counts sputum preparations were fixed and stained using a commercially available kit based on May-Grünewald Giemsa stain (Surgipath Industries, Richmond, Ill., USA).

### Microbiology

Microbiology is the cross-sectional analysis of V1 assays, but not in the analysis of longitudinal change.

### Measurement of Sputum and Serum Protein Biomarkers

Commercially available ELISAs were used to measure IL-8 (IL-8 Easia Kit, Biosource, UK), MPO (Assay Designs, Michigan, USA), and CRP (Quantikine, R and D Systems, Oxford, UK). IL-1 β, IL-6, IL-10, IL-12, RANTES, TNF-α and MMP-9 were measured using a cytometric bead array (Luminex). Calprotectin was measured by an in-house double antibody sandwich ELISA, using monoclonal and polyclonal antibodies against human calprotectin (gift of Erling Sundrehagen, Norway).

### Computed Tomography

Inspiratory and expiratory CT scans were performed without intravenous contrast: (a) inspiratory volumetric contiguous thin-sections (1 mm) through the entire volume of the lungs; (b) end expiratory interspaced high resolution CT at expiration with 1 mm sections every 10 mm. Scans were performed on a Siemens 64-slice multi-detector scanner (Siemens AG, Erlangemn, Germany) at 2 sites (RBH & RHSC) and a Siemens Sensation 16-slice scanner at the third site (WGH). To limit effective dose, 100 kVp was used for both scans with weight adjusted mAs values: 1 mAs per kg<30 kg, 30 mAs 30-50 kg, 35 mAs>50 kg, 50 mAs>70 kg.

Anonymised CT images were saved to a compact disc for scoring by two independent radiologists. CTs were scored for eight independent features using a scoring system. The scoring system provides independent scores for each of the eight parameters and no global score is calculated. Bronchiectasis, bronchial wall thickness, small and large airway mucus plugging were assessed for extent and severity using a semi-quantitative graded scoring system. Air trapping, consolidation and ground glass opacification were scored as a percentage of lung affected (to the nearest 5%), in each of the six. The final score represents the sum of the individual lobe scores for that feature from both radiologists, i.e. 12× the maximum score (2 radiologists×6 lobes) possible for an individual lobe. To facilitate comparison between variables, we have expressed as a percentage of the maximum possible score.

### Statistical Analysis

Data were analysed using Prism (GraphPad Software Inc, CA, USA). Normal distribution was assessed using the D'Agostino and Pearson omnibus normality test. Results are quoted as mean (SD) or median (interquartile range) unless otherwise stated. Non-parametric data were log-transformed prior to analysis. Paired t-test was used for comparison of change in variables between paired visits. Comparisons between multiple groups were performed using a one way ANOVA (parametric data) or Kruskal Wallis (non-parametric). Lower limit of detection for the CRP assay was 3 mg/ml (Edinburgh samples) or 1 mg/ml (London samples). Levels reported as below the lower limit have all been ascribed a value of 1 mg/ml. Likewise, MMP9 levels reported as below the lower limit of detection of 50 ng/ml have been assigned a value of 50 ng/ml.

Correlations between different assays were performed on assessments performed at visit 1, and included all those with valid assessments at that visit even if subsequent assessments were missing or excluded because of protocol violation. Correlations were assessed using the Pearson correlation coefficient (parametric data) or Spearman R (non-parametric). A p value of below 0.05 was considered as statistically significant

### Results

### Patient Demographics and Clinical Characteristics

Forty-six patients consented to the study. Two patients were subsequently excluded from all analyses for concomitant use of oral corticosteroids and cross sectional data correlations have therefore been performed on 44 patients. A further six patients were excluded from longitudinal analysis because of an excessive time delay (>5 days) (n=2) or non-attendance (n=3) at visit 2, or because of commencing oral corticosteroids between visits 1 and 2 (n=1). Assessment of longitudinal change in assays was performed on the remaining 38 patients.

Median age [range] of the 44 patients (20 female) who completed visit 1 was 23 [11-44] years. Demographic data are summarised in Table 6. Mean (SD, range) FEV₁ Z score at start of treatment was −4.29 (1.03, −1.59 to −6.13), or 52.1 (12.2, 29.6 to 86.8) percent predicted (n=42). 27 (61%) of patients were phe508del homozygotes, and 16 (36%) were phe508del heterozygotes. A single subject had no copies of the phe508del gene (genotype G551D/1717-1G-7A). Patient characteristics noted at time of commencing IV antibiotics were N (%); increased cough 43 (98%); increased dyspnoea 41 (93%); change in sputum 39 (89%); malaise 37 (84%); fall in FEV₁>10% 19 (43%).

TABLE 6
Demographics and symptoms at start of treatment.

| | |
|---|---|
| Number of subjects | 44 |
| Sex (m/f) | 24/20 |
| Median [range] age (yrs) | 23 [11-44] |
| Characteristics of exacerbation: N (%) | |
| Increased cough | 43 (98) |
| Increased dyspnoea | 41 (93) |
| Change in sputum | 39 (89) |
| Malaise | 37 (84) |
| Fall in FEV₁ > 10% | 19 (43) |
| Mean (SD) FEV₁ at start of treatment: Z score | −4.29 (1.03) |
| [% predicted] | 52.1 (12.2) |

### Longitudinal Analysis

Valid paired assessments were available on 38 patients (16 female). A summary of the change in individual assays is given in table presented in FIG. 23. All patients were treated with a minimum of two combined intravenous antibiotics for a median [range] treatment duration of 14 [9-24] days. Treatment choice was at the discretion of the clinical team.

Thirty six (95%) of V1 were performed within 24 hours of starting IV antibiotics and 31 (82%) of V2s within 48 hours of completion of IV antibiotics.

### 1. Symptoms and Clinical Physiology

Baseline symptom score was less than zero in 36 out of 37 (97%) patients, confirming that patients felt worse than usual at the start of treatment. Following treatment, mean (SD) total symptom score improved by an average of 9.5 points from −6.7 (3.0) to 2.8 (5.6) (p<0.0001) (FIG. 17).

There were small, but statistically significant, changes with treatment in mean respiratory rate, heart rate diastolic blood pressure and weight (see table presented in FIG. 23). No significant changes were observed in mean systolic blood pressure or transcutaneous oxygen saturations; the latter was normal at baseline in all patients and no subject required additional O₂ (if correct)

### 2. Lung Physiology

Mean (SD) FEV₁ z score improved from −4.03 (1.10) to −3.23 (1.42), p=0.0009, FIG. 18. FEV₁% predicted increased by a mean (SD) of 9.6 (14.6) percent predicted to 64.6 (16.8) percent predicted at the end of treatment. There was a mean (SD) improvement in FVC z score of 0.93 (1.36), p=0.003 but no significant change in FEF₂₅₋₇₅ (p=0.13).

Mean (SD) FEV₁ at the end of treatment was similar to patients' best recorded FEV₁ within the last 6 months for the group as a whole: 2.25 (0.76) L at end of treatment vs. 2.22 (0.86) L as recent best, p=0.8. However, the degree of change in FEV₁ with treatment was related to the severity of the fall in FEV₁ from recent best at start of IV antibiotics. Patients with an FEV₁ fall of <10% at study entry (n=17) improved by a median of 7.2% to 6.4% above baseline. Where FEV₁ fall was >10% at study entry, improvement was by a median of 31% (p=0.05 compared to those with no significant fall in FEV₁ at study entry), but remained 13% below baseline at the end of treatment (p=0.02). Differential improvements in FEV₁ were not reflected by differential improvements in LCI.

Mean (SD) LCI improved following antibiotics from 14.6 (2.7) to 13.8 (2.4), p=0.003, FIG. 18b. There was no significant change in mean (SD) FRC; 2.32 (0.58) vs. 2.33 (0.62) L (p=0.8).

### 3. Structure

Significant improvement (mean, sd) was observed from the start to end of treatment for airway wall thickness [54.0 (11.3) vs. 49.5 (10.8) percent of maximum possible score, p=0.008], small mucus plugs [78.5 (16.8) vs. 69.6 (20.6) %, p=0.018], large mucus plugs [72.0 (22.0) vs. 59.0 (23.5) %, p<0.0001] and air trapping [48.5 (16.1) vs. 40.8 (13.4) %, p=0.004] (FIG. 19). Although the change in lung consolidation score was statistically significant (p=0.03), this was not a prominent feature of the CT scans, with an average of only 23, or 1.9% of the maximum score, at visit 1. No significant changes were observed for ground glass opacification, and extent and severity of bronchiectasis (p>0.05).

### 4. Serum Inflammatory Markers

Four serum markers of systemic inflammation improved following treatment with IV antibiotics: plasma WCC [mean (SD)][10.2 (2.6) at start of treatment vs. 8.71 (3.16) at end, p=0.02]; CRP [38.2 (52.9) vs. 10.3 (22.3) mg/L, p<0.0001]; IL-6 [68.7 (1.4) vs. 51.5 (1.1) pg/ml, p<0.0001) and calprotectin [31.6 (2.4) vs. 12.3 (2.4) ng/ml, p<0.0001], see FIG. 4. No significant changes were observed for IL-8 or TNF-α levels. Serum IL-10 and IL-1β were generally undetectable, and no useful information was gained from these assays.

### 5. Sputum Inflammatory Markers

There was a significant reduction in median 24-hour sputum weight from 60.3 g to 34.0 g, (p=0.04). Total sputum cell count fell from a median of 5.3×10⁻⁶/ml to 2.1×10⁻⁶/ml (p=0.005) after treatment, but there was no significant change in the sputum differential cell counts expressed as % of total. There were significant reductions in the level of sputum MMP9 (470 ng/ml at start of treatment vs 214 ng/ml at end, p=0.006) and IL-1β (1032 pg/ml vs 410 pg/ml, p=0.01) see FIG. 21, but no significant change was seen in the other sputum cytokines (neutrophil elastase, MPO, IL-12 and IL-8). In contrast to the changes seen in serum levels, there was also no significant change in the sputum levels of calprotectin. IFN-γ was generally undetectable in sputum, and no useful information was gained from this assay.

### Correlations Between Measurements at Baseline (VI)

Markers with the most significant change between visits 1 and 2 were selected as being of principal interest for cross sectional analysis of correlations. A correlation “mileage chart” of these markers is presented in Table 7. In addition, these markers are compared to all the remaining assays in a larger mileage chart presented in the table shown in Table 8.
Symptom score correlated weakly but significantly with the serum inflammatory markers CRP, IL-6 and calprotectin but not with any of the clinical markers of respiratory function or physiology except for FVC, nor did it correlate with any of the individual radiological markers.
FEV₁ correlated well with other markers of lung physiology (FVC, FEF and LCI), and also with the following radiological markers of airway disease: airway wall thickness, mucus plugging and gas trapping.
LCI correlated with the physiological and radiological markers of airways disease (FEV₁, FEF, air trapping and small and large mucus plugging). The serum inflammatory markers CRP, IL-6 and calprotectin all correlated with each other, but calprotectin also showed the most convincing correlations with other measures of severity, including symptom score, spirometry and LCI. Sputum MMP9 only correlated significantly with other measures of sputum inflammation (calprotectin, IL-8 and MPO), but not with any of the other assays. Two markers, calprotectin and IL-8, were measured in both serum and sputum. In both cases, there was no correlation between the sputum and serum levels.

TABLE 7
Mileage chart of correlations between different markers of disease activity at
start of exacerbation. Numbers represent individual Pearson correlation r scores.
Significant correlations are highlighted and marked with a *.
^{‡}Log transformed data.

| | | | | Airway wall | | | | Serum |
|---|---|---|---|---|---|---|---|---|
| Assay | Symptom score | FEV₁ SDS | LCI | thick. | Air Trapping | Serum CRP | Serum IL-6 | Calprotectin |
| FEV₁ SDS | 0.31 | 1.0 | | | | | | |
| LCI | −0.03 | −0.52* | 1.0 | | | | | |
| Airway wall thickness | −0.14 | −0.51* | 0.15 | 1.0 | | | | |
| Air trapping | −0.11 | −0.31* | 0.48* | 0.03 | 1.0 | | | |
| Serum CRP (mg/ml)^{‡} | −0.55* | −0.25 | −0.09 | 0.29 | 0.32 | 1.0 | | |
| Serum IL-6 (pg/ml)^{‡} | −0.40* | −0.24 | −0.17 | −0.13 | 0.35 | 0.52* | 1.0 | |
| Serum Calpro. (ng/ml)^{‡} | −0.33* | −0.39* | 0.34 | 0.23 | 0.32 | 0.67* | 0.41* | 1.0 |
| Sputum MMP9 (ng/ml)^{‡} | −0.08 | 0.29 | −0.11 | −0.13 | 0.10 | 0.12 | 0.10 | 0.23 |

TABLE 8
Mileage chart of correlations between different markers of disease activity at
start of exacerbation. Correlations are shown between the 9 assays with most significant
change with treatment, against all other markers assayed. Numbers represent individual
Pearson correlation r scores. Significant correlations are highlighted and marked with a *.
^{‡}Log transformed data.

| | | Symptom | | | | | | | Serum | Sputum |
|---|---|---|---|---|---|---|---|---|---|---|
| Assay | | score | FEV₁ | LCI | AWT | Air Trap | CRP | Serum IL6 | Calprotetcin | MMP9 |
| Symptoms/ | Symptom score | x | 0.31 | −0.03 | −0.14 | −0.11 | −0.55* | −0.40* | −0.33* | −0.08 |
| Clinical | Weight (kg) | −0.18 | 0.20 | 0.00 | −0.08 | 0.11 | 0.10 | 0.11 | 0.19 | 0.15 |
| | Heart rate(min⁻¹) | −0.21 | −0.29 | 0.10 | 0.09 | 0.12 | 0.26 | 0.28 | 0.09 | 0.04 |
| | Resp. rate (min⁻¹) | 0.23 | 0.13 | −0.22 | −0.37* | 0.00 | −0.05 | 0.24 | −0.09 | −0.21 |
| | O₂ saturation | 0.25 | 0.14 | −0.32* | 0.11 | −0.62* | −0.30 | −0.07 | −0.19 | −0.11 |
| | Syst. BP (mmHg) | 0.32* | 0.17 | 0.19 | −0.26 | −0.05 | −0.26 | −0.24 | −0.08 | 0.07 |
| | Diast. BP (mmHg) | 0.31* | 0.20 | 0.08 | 0.08 | −0.03 | −0.36* | −0.48* | −0.32* | −0.12 |
| Function | FEV₁ SDS | 0.31 | x | −0.52* | −0.51* | −0.31* | −0.25 | −0.24 | −0.39* | 0.29 |
| | LCI | −0.03 | −0.52* | x | 0.15 | 0.48* | −0.09 | −0.17 | 0.34* | −0.11 |
| | FVC SDS | 0.31* | 0.55* | −0.16 | −0.48* | −0.05 | −0.29 | −0.22 | −0.40* | 0.19 |
| | FEF₂₅₋₇₅ SDS | 0.21 | 0.72* | −0.68* | −0.56 | −0.39 | −0.16 | −0.26 | −0.30 | 0.27 |
| | FRC (L) | 0.08 | −0.15 | 0.19 | −0.23 | 0.10 | −0.15 | −0.06 | 0.03 | 0.27 |
| Structure | Extent bronchiect. | 0.02 | −0.29 | 0.27 | 0.52* | 0.03 | 0.21 | −0.17 | 0.29 | −0.06 |
| | Severity bronc. | 0.19 | −0.26 | 0.05 | 0.74* | −0.29 | −0.12 | −0.38* | 0.05 | −0.17 |
| | Airwy wall thick. | −0.14 | −0.51* | 0.15 | x | 0.03 | 0.29 | −0.13 | 0.23 | −0.13 |
| | Air trapping | −0.11 | −0.31* | 0.48* | 0.03 | x | 0.32 | 0.35 | 0.32 | 0.10 |
| | Small mucus plugs | −0.20 | −0.35* | −0.38* | 0.16 | 0.33 | 0.50* | 0.31 | 0.30 | 0.03 |
| | Large mucus plugs | −0.16 | −0.41* | −0.45* | 0.72 | 0.08 | 0.31 | 0.03 | 0.30 | 0.10 |
| | Lung consolidation | −0.25 | −0.28 | 0.00 | 0.30 | 0.05 | 0.35 | −0.09 | 0.30 | −0.19 |
| | Ground glass lung | −0.11 | −0.32 | −0.01 | 0.24 | 0.20 | 0.40 | 0.13 | 0.69* | 0.01 |
| Serum | WCC | −0.19 | −0.26 | 0.28 | 0.14 | 0.41* | 0.22 | 0.24 | 0.60* | 0.09 |
| | CRP (mg/ml)^{‡} | −0.55* | −0.25 | −0.09 | 0.29 | 0.32 | x | 0.52* | 0.67* | 0.12 |
| | IL-6 (pg/ml)^{‡} | −0.40* | −0.24 | −0.17 | −0.13 | 0.35 | 0.52* | x | 0.41* | 0.10 |
| | Calpro. (ng/ml)^{‡} | −0.33* | −0.39* | 0.34 | 0.23 | 0.32 | 0.67* | 0.41* | x | 0.23 |
| | IL-8^{‡} | 0.14 | 0.29 | −0.24 | −0.20 | −0.03 | −0.08 | −0.05 | −0.13 | −0.06 |
| | TNF-α | 0.15 | 0.12 | −0.36* | −0.25 | 0.01 | −0.22 | 0.37* | 0.04 | 0.07 |
| Sputum | 24-hr weight (g)^{‡} | −0.09 | −0.45* | 0.46* | 0.44* | 0.07 | 0.06 | −0.01 | 0.37 | 0.03 |
| | Tt. cell ct. (×10⁻⁶)^{‡} | −0.27 | −0.26 | 0.30 | 0.27 | 0.27 | 0.21 | 0.10 | 0.28 | 0.21 |
| | IL-12 (pg/ml) | 0.28 | 0.14 | −0.28 | −0.06 | −0.32 | 0.06 | −0.07 | −0.12 | −0.23 |
| | MMP9 (ng/ml)^{‡} | −0.08 | 0.29 | −0.11 | −0.13 | 0.10 | 0.12 | 0.10 | 0.23 | x |
| | Calpro. (ng/ml)^{‡} | −0.14 | −0.16 | 0.06 | 0.22 | 0.52* | 0.33* | 0.30 | 0.22 | 0.57* |
| | IL-8 (pg/ml) | 0.15 | 0.14 | 0.13 | −0.10 | 0.15 | −0.13 | −0.29 | 0.00 | 0.51* |
| | Neut. Elas. (μg/ml) | −0.06 | −0.07 | 0.17 | −0.14 | 0.37* | 0.18 | 0.25 | 0.20 | 0.08 |
| | MPO (μg/ml)^{‡} | −0.14 | −0.19 | 0.28 | 0.15 | 0.37* | 0.20 | 0.09 | 0.20 | 0.39* |
| | IL-1β‡ | −0.11 | −0.15 | 0.29 | 0.27 | 0.45* | 0.06 | 0.22 | 0.17 | 0.29 |

The table shown in FIG. 24 shows a correlation mileage chart of changes in markers of disease activity over the course of treatment for an exacerbation of CF lung disease. Correlations are shown between changes in markers with significant change, against selected markers with the most significant change and clinical relevance. For sputum and serum markers of inflammation, the changes are in the logged values. Numbers represent individual Pearson correlation r scores above p value for this correlation. Non-parametric data assessed by Spearman rank correlation test. Numbers highlighted in bold italics represent statistically significant change (p<0.05).

### Discussion

This is the broadest study of its kind in that it to simultaneously and interactively assessed a comprehensive range of established and novel biomarkers in CF. In addition, we have assessed these longitudinally, in response to a course of IV antibiotics, as a model for the effects of a therapeutically beneficial intervention. We identified that clinical recovery in association with treatment is reflected by improvements in all five of the domains examined, with significant improvements in symptoms, lung function, CT measures of mucus plugging and airway wall thickness, certain systemic inflammatory markers and selected sputum inflammatory markers. These results are important not simply in describing the pathophysiology of CF exacerbations, but also in exploring the relationship between variables and assessing the usefulness of the biomarkers as end points in studies of novel therapeutic agents.

Perhaps the most striking improvement seen was in patient symptoms—the majority (61%) of patients felt better than their normal baseline at the end of treatment. This was despite FEV₁ improving to little more than the level of recent baseline and only minor improvements with treatment in routine clinical measurements, such as oxygen saturations. Improvement in symptom score did correlate with improved measures of sputum load however, such as sputum 24 hr weight and large airway plugs, as well as FEV₁, airway wall thickness and CRP. There are two key messages that can be drawn from these observations. The first is that antibiotic treatment clearly has significant effects both on patient symptoms and spirometry. The effect on symptom, spirometry and LCI correlates best with the change of large airway secretions ie. may be a proximal airway effect. The second important observation is that there is divergence between how patients feel and many clinical parameters. Despite much effort over the last few years in identifying potential markers of treatment response in CF, it appears to be the effects of increased sputum that patients' symptoms and lung function are most influenced by. The complexity of patient symptoms may be compounded by effects of the severity of the underlying lung disease. Despite increasing interest in defining symptoms, and exacerbations, this remains a poorly understood area. Short term changes (or lack of change) in symptoms may not reflect longer term outcomes.

LCI, as a measure of overall ventilation heterogeneity, will be affected by both fixed abnormalities in airway and parenchymal structure due to fibrotic and destructive processes, as well as reversible abnormalities due to regional differences in inflammation and mucus retention. There is considerable heterogeneity of response to IV antibiotics, with some subjects even showing an increase (worsening) in LCI. This suggests that some less well ventilated lung regions may be opened up by treatment as mucus is cleared, causing overall inhomogeneity (and thus LCI) to increase. In vivo, the interactions between different lung units are not independent, and the effects on LCI and FRC of mucus clearance likely to be complex and unpredictable. More consistent improvement in LCI has been reported in patients with milder disease in response to nebulised hypertonic saline and DNase, and LCI may be a more useful measurement in stable patients with milder disease where large shifts in airway mucus are less of a feature than this relatively sever cohort.

Changes on CT have previously been shown to correlate with important clinical outcomes, such as pulmonary exacerbations, and to correlate with regional inflammation. The novel CT scoring system used here has previously been shown to have good reproducibility and involves a more detailed assessment of individual CT features than some of the composite scores; in our opinion and in contrast to global scores, it also allows separation of fixed (eg bronchiectasis) from potential reversible (eg wall thickness parameters) preventing a change in the latter being diluted by a lack of change in the former. Although the use of this score does not permit direct comparison with previous studies, the findings for mucus plugging are in line with those previously reported, although we also observed improvements in air trapping and wall thickness, which may reflect the sensitivity of the scoring system or the resolution of the scans.

IL-8 is has long been regarded as one of the major chemo-attractants in CF airways, and is known to be induced by both elastase and TNFα. Sputum IL-8 has been shown to correlate with FEV₁ in a large cross sectional analysis, combining 269 patients from 4 different studies, and has been identified as a candidate biomarker of CF airways inflammation. However, despite being elevated in CF patients, we found no significant change in either sputum or serum IL8, and no correlation between the two. The failure of IL-8 to improve with therapy is in agreement with previously published observations, and casts a shadow over its potential as a useful biomarker in interventional studies.

The most significant changes in markers of inflammation were observed in serum rather than sputum: CRP, a non-specific marker of inflammation, and calprotectin, a marker of neutrophillic inflammation previously shown to be elevated in CF. These showed greater change than either sputum or serum cell counts, or any of the sputum biological markers of inflammation, in line with previous observations. The high levels of CRP at the start of treatment indicate a significant degree of inflammatory response, and one that is expressed beyond the confines of the lung. It seems unlikely that this would have been insufficient to cause change in the levels of individual chemokines if they were to be useful indicators of the level of inflammation. But likely less useful biomarkers in long term study in stable patients.

The definition of exacerbation was a clinical one, decided upon by the treating clinician. There is also no way of ensuring full compliance with treatment for patients treated at home. However, the significant improvements seen in a number of markers indicate both that the majority of patients were unwell at the start of treatment and also that they had improved in response to antibiotics.

While data is incomplete for some of the analyses, the majority contained data on at least 30 pairs. It was unfortunate that spirometry was lacking at one or other time point on some subjects, and we have used the spirometry from the Piko device where available and where it has been shown to be reliably reproducible. Since mid expiratory flow and FVC are not generated by the Piko, we cannot use this as a substitute, and these datasets remain incomplete.

Both the symptom score and CT score are novel measures, not previously subject to widespread external validation. The symptom score was designed to be straightforward and to reflect major symptoms. Unlike conventional quality of life scores, it is a comparative score, comparing patients' current symptoms to their usual baseline, and no weighting is given to any individual symptoms. Although a number of different scores have been used in previous studies to assess acute change with antibiotics, at the time the study was conceived none had been subjected to a formal evaluation process and there was no accepted gold standard assessment tool designed specifically for assessing acute symptom change in CF, which would have been clearly superior to own score.

In this study patients were unwell at the start—this is a different scenario to that found in clinical trials when the usual approach is to treat stable patients, with the aim of detecting an improvement above their usual baseline. However, it is reasonable to anticipate that markers should improve with clinical status if they are to prove useful measures of treatment success. To this end, encouraging improvements were have been seem in a number of novel parameters in addition to those that might be anticipated in FEV₁, symptoms and serum CRP. Although the treatment effects are not necessarily comparable to that which might be obtained from stable patients the baseline data on population means, and spread of data, will assist power calculations for future studies. In addition, all the assays have been shown to be suitable for multi-centre assessments, many of which have previously been restricted to single centre studies. This was in itself an enormous logistic challenge, and a vital step in establishing robust clinical research networks and multi-centre trials.

A particular problem with assessing novel interventions in CF is that the falling rate of decline in lung function, and improvements in survival, mean that these parameters are no longer appropriate end points for the majority of studies. Since the small airways are believed to play such an important role in either initiation or progression of disease, it is vital that we develop more specific biomarkers of disease activity in this region. Current measurements of lung function are insensitive to small airway function (REF Horsley, Aurora), and in this study FEV₁ and symptoms both appear to be poor surrogates for changes in this region of the lung.

### EXAMPLE 13

### Further Assessment of the Physical Stability of GL67A/pDNA Formulations During Nebulization

The present inventors sought to examine the stability of the GL67A/pGM169 complexes. This data is in addition to that presented in Example 3 provided above.

The data presented in FIG. 25 concerns vector-specific CFTR expression after GL67A/pGM169 transfection, ratioed to endogenous CFTR expression in fully differentiated human air liquid interface cultures (ALIs). In this experiment, ALIs were transfected with GL67A/pGM169, the investigational medicinal production (IMP) or remained untransfected (UT). 24 hrs post-transfection, vector-specific and endogenous CFTR mRNA were quantified using quantitative RT-PCR. The data provided in this figure shows that GL67A transfects differentiated human airway epithelium efficiently and produces vector-specific CFTR mRNA at approximately 10% of endogenous levels.

In addition, the data presented in FIG. 26 concerns the integrity of GL67A complexed pGM169 in the AeroEclipse II nebuliser. In panel (A), pGM169 plasmid integrity was assessed by gel electrophoresis pre nebulisation and after 5, 10 and 15 min (end of nebulisation) of nebulisation. Red arrow indicates supercoiled Plasmid DNA. In panel (B), at the same time-point after nebulisation chloride transport mediated by the vector-specific CFTR protein was assessed after in vitro transfection of cells with GL67A/pGM169 complexes collected from AeroEclipse II nebulisers. The data provide in this figure shows that, although some plasmid degradation occurs in the nebuliser, a significant amount of supercoiled plasmid DNA is present at the end of nebulisation. Importantly, this plasmid DNA generates CFTR protein leading to efficient ion transport. Importantly, nebulisation does not reduce efficacy of ion transport.

In addition, the data presented in FIG. 26 concerns an assessment of chloride transport mediated by vector-specific CFTR protein after in vitro transfection of cells with GL67A/pGM169 complexes collected from AeroEclipse II nebulisers prior to nebulisation to patients and after transfection with residual complexes left over in the nebulisers at the end of nebulisation. The data provide in this figure shows that throughout the nebulisation process GL67A/pGM169 is able to generate CFTR protein which supports ion transport and therefore indicates that the IMP is stable in the AeroEclipse II nebulisers.

| Nucleic acid sequences | |
|---|---|
| (pGM169) | |
| SEQ ID NO: 1 | |
| 1 | AGATCTGTTA CATAACTTAT GGTAAATGGC CTGCCTGGCT GACTGCCCAA TGACCCCTGC |
| 61 | CCAATGATGT CAATAATGAT GTATGTTCCC ATGTAATGCC AATAGGGACT TTCCATTGAT |
| 121 | GTCAATGGGT GGAGTATTTA TGGTAACTGC CCACTTGGCA GTACATCAAG TGTATCATAT |
| 181 | GCCAAGTATG CCCCCTATTG ATGTCAATGA TGGTAAATGG CCTGCCTGGC ATTATGCCCA |
| 241 | GTACATGACC TTATGGGACT TTCCTACTTG GCAGTACATC TATGTATTAG TCATTGCTAT |
| 301 | TACCATGGGA ATTCACTAGT GGAGAAGAGC ATGCTTGAGG GCTGAGTGCC CCTCAGTGGG |
| 361 | CAGAGAGCAC ATGGCCCACA GTCCCTGAGA AGTTGGGGGG AGGGGTGGGC AATTGAACTG |
| 421 | GTGCCTAGAG AAGGTGGGGC TTGGGTAAAC TGGGAAAGTG ATGTGGTGTA CTGGCTCCAC |
| 481 | CTTTTTCCCC AGGGTGGGGG AGAACCATAT ATAAGTGCAG TAGTCTCTGT GAACATTCAA |
| 541 | GCTTCTGCCT TCTCCCTCCT GTGAGTTTGG TAAGTCACTG ACTGTCTATG CCTGGGAAAG |
| 601 | GGTGGGCAGG AGATGGGGCA GTGCAGGAAA AGTGGCACTA TGAACCCTGC AGCCCTAGGA |
| 661 | ATGCATCTAG ACAATTGTAC TAACCTTCTT CTCTTTCCTC TCCTGACAGG TTGGTGTACA |
| 721 | GTAGCTTGCT AGCCACCATG CAGAGAAGCC CTCTGGAGAA GGCCTCTGTG GTGAGCAAGC |
| 781 | TGTTCTTCAG CTGGACCAGG CCCATCCTGA GGAAGGGCTA CAGGCAGAGA CTGGAGCTGT |
| 841 | CTGACATCTA CCAGATCCCC TCTGTGGACT CTGCTGACAA CCTGTCTGAG AAGCTGGAGA |
| 901 | GGGAGTGGGA TAGAGAGCTG GCCAGCAAGA AGAACCCCAA GCTGATCAAT GCCCTGAGGA |
| 961 | GATGCTTCTT CTGGAGATTC ATGTTCTATG GCATCTTCCT GTACCTGGGG GAAGTGACCA |
| 1021 | AGGCTGTGCA GCCTCTGCTG CTGGGCAGAA TCATTGCCAG CTATGACCCT GACAACAAGG |
| 1081 | AGGAGAGGAG CATTGCCATC TACCTGGGCA TTGGCCTGTG CCTGCTGTTC ATTGTGAGGA |
| 1141 | CCCTGCTGCT GCACCCTGCC ATCTTTGGCC TGCACCACAT TGGCATGCAG ATGAGGATTG |
| 1201 | CCATGTTCAG CCTGATCTAC AAGAAAACCC TGAAGCTGTC CAGCAGAGTG CTGGACAAGA |
| 1261 | TCAGCATTGG CCAGCTGGTG AGCCTGCTGA GCAACAACCT GAACAAGTTT GATGAGGGCC |
| 1321 | TGGCCCTGGC CCACTTTGTG TGGATTGCCC CTCTGCAGGT GGCCCTGCTG ATGGGCCTGA |
| 1381 | TTTGGGAGCT GCTGCAGGCC TCTGCCTTTT GTGGCCTGGG CTTCCTGATT GTGCTGGCCC |
| 1441 | TGTTTCAGGC TGGCCTGGGC AGGATGATGA TGAAGTACAG GGACCAGAGG GCAGGCAAGA |
| 1501 | TCAGTGAGAG GCTGGTGATC ACCTCTGAGA TGATTGAGAA CATCCAGTCT GTGAAGGCCT |
| 1561 | ACTGTTGGGA GGAAGCTATG GAGAAGATGA TTGAAAACCT GAGGCAGACA GAGCTGAAGC |
| 1621 | TGACCAGGAA GGCTGCCTAT GTGAGATACT TCAACAGCTC TGCCTTCTTC TTCTCTGGCT |
| 1681 | TCTTTGTGGT GTTCCTGTCT GTGCTGCCCT ATGCCCTGAT CAAGGGGATC ATCCTGAGAA |
| 1741 | AGATTTTCAC CACCATCAGC TTCTGCATTG TGCTGAGGAT GGCTGTGACC AGACAGTTCC |
| 1801 | CCTGGGCTGT GCAGACCTGG TATGACAGCC TGGGGGCCAT CAACAAGATC CAGGACTTCC |
| 1861 | TGCAGAAGCA GGAGTACAAG ACCCTGGAGT ACAACCTGAC CACCACAGAA GTGGTGATGG |
| 1921 | AGAATGTGAC AGCCTTCTGG GAGGAGGGCT TTGGGGAGCT GTTTGAGAAG GCCAAGCAGA |
| 1981 | ACAACAACAA CAGAAAGACC AGCAATGGGG ATGACTCCCT GTTCTTCTCC AACTTCTCCC |
| 2041 | TGCTGGGCAC ACCTGTGCTG AAGGACATCA ACTTCAAGAT TGAGAGGGGG CAGCTGCTGG |
| 2101 | CTGTGGCTGG ATCTACAGGG GCTGGCAAGA CCAGCCTGCT GATGATGATC ATGGGGGAGC |
| 2161 | TGGAGCCTTC TGAGGGCAAG ATCAAGCACT CTGGCAGGAT CAGCTTTTGC AGCCAGTTCA |
| 2221 | GCTGGATCAT GCCTGGCACC ATCAAGGAGA ACATCATCTT TGGAGTGAGC TATGATGAGT |
| 2281 | ACAGATACAG GAGTGTGATC AAGGCCTGCC AGCTGGAGGA GGACATCAGC AAGTTTGCTG |
| 2341 | AGAAGGACAA CATTGTGCTG GGGGAGGGAG GCATTACACT GTCTGGGGGC CAGAGAGCCA |
| 2401 | GAATCAGCCT GGCCAGGGCT GTGTACAAGG ATGCTGACCT GTACCTGCTG GACTCCCCCT |
| 2461 | TTGGCTACCT GGATGTGCTG ACAGAGAAGG AGATTTTTGA GAGCTGTGTG TGCAAGCTGA |
| 2521 | TGGCCAACAA GACCAGAATC CTGGTGACCA GCAAGATGGA GCACCTGAAG AAGGCTGACA |
| 2581 | AGATCCTGAT CCTGCATGAG GGCAGCAGCT ACTTCTATGG GACCTTCTCT GAGCTGCAGA |
| 2641 | ACCTGCAGCC TGACTTCAGC TCTAAGCTGA TGGGCTGTGA CAGCTTTGAC CAGTTCTCTG |
| 2701 | CTGAGAGGAG GAACAGCATC CTGACAGAGA CCCTGCACAG ATTCAGCCTG GAGGGAGATG |
| 2761 | CCCCTGTGAG CTGGACAGAG ACCAAGAAGC AGAGCTTCAA GCAGACAGGG GAGTTTGGGG |
| 2821 | AGAAGAGGAA GAACTCCATC CTGAACCCCA TCAACAGCAT CAGGAAGTTC AGCATTGTGC |
| 2881 | AGAAAACCCC CCTGCAGATG AATGGCATTG AGGAAGATTC TGATGAGCCC CTGGAGAGGA |
| 2941 | GACTGAGCCT GGTGCCTGAT TCTGAGCAGG GAGAGGCCAT CCTGCCTAGG ATCTCTGTGA |
| 3001 | TCAGCACAGG CCCTACACTG CAGGCCAGAA GGAGGCAGTC TGTGCTGAAC CTGATGACCC |
| 3061 | ACTCTGTGAA CCAGGGCCAG AACATCCACA GGAAAACCAC AGCCTCCACC AGGAAAGTGA |
| 3121 | GCCTGGCCCC TCAGGCCAAT CTGACAGAGC TGGACATCTA CAGCAGGAGG CTGTCTCAGG |
| 3181 | AGACAGGCCT GGAGATTTCT GAGGAGATCA ATGAGGAGGA CCTGAAAGAG TGCTTCTTTG |
| 3241 | ATGACATGGA GAGCATCCCT GCTGTGACCA CCTGGAACAC CTACCTGAGA TACATCACAG |
| 3301 | TGCACAAGAG CCTGATCTTT GTGCTGATCT GGTGCCTGGT GATCTTCCTG GCTGAAGTGG |
| 3361 | CTGCCTCTCT GGTGGTGCTG TGGCTGCTGG GAAACACCCC ACTGCAGGAC AAGGGCAACA |
| 3421 | GCACCCACAG CAGGAACAAC AGCTATGCTG TGATCATCAC CTCCACCTCC AGCTACTATG |
| 3481 | TGTTCTACAT CTATGTGGGA GTGGCTGATA CCCTGCTGGC TATGGGCTTC TTTAGAGGCC |
| 3541 | TGCCCCTGGT GCACACACTG ATCACAGTGA GCAAGATCCT CCACCACAAG ATGCTGCACT |
| 3601 | CTGTGCTGCA GGCTCCTATG AGCACCCTGA ATACCCTGAA GGCTGGGGGC ATCCTGAACA |
| 3661 | GATTCTCCAA GGATATTGCC ATCCTGGATG ACCTGCTGCC TCTCACCATC TTTGACTTCA |
| 3721 | TCCAGCTGCT GCTGATTGTG ATTGGGGCCA TTGCTGTGGT GGCAGTGCTG CAGCCCTACA |
| 3781 | TCTTTGTGGC CACAGTGCCT GTGATTGTGG CCTTCATCAT GCTGAGGGCC TACTTTCTGC |
| 3841 | AGACCTCCCA GCAGCTGAAG CAGCTGGAGT CTGAGGGCAG AAGCCCCATC TTCACCCACC |
| 3901 | TGGTGACAAG CCTGAAGGGC CTGTGGACCC TGAGAGCCTT TGGCAGGCAG CCCTACTTTG |
| 3961 | AGACCCTGTT CCACAAGGCC CTGAACCTGC ACACAGCCAA CTGGTTCCTC TACCTGTCCA |
| 4021 | CCCTGAGATG GTTCCAGATG AGAATTGAGA TGATCTTTGT CATCTTCTTC ATTGCTGTGA |
| 4081 | CCTTCATCAG CATTCTGACC ACAGGAGAGG GAGAGGGCAG AGTGGGCATT ATCCTGACCC |
| 4141 | TGGCCATGAA CATCATGAGC ACACTGCAGT GGGCAGTGAA CAGCAGCATT GATGTGGACA |
| 4201 | GCCTGATGAG GAGTGTGAGC AGAGTGTTCA AGTTCATTGA TATGCCCACA GAGGGCAAGC |
| 4261 | CTACCAAGAG CACCAAGCCC TACAAGAATG GCCAGCTGAG CAAAGTGATG ATCATTGAGA |
| 4321 | ACAGCCATGT GAAGAAGGAT GATATCTGGC CCAGTGGAGG CCAGATGACA GTGAAGGACC |
| 4381 | TGACAGCCAA GTACACAGAG GGGGGCAATG CTATCCTGGA GAACATCTCC TTCAGCATCT |
| 4441 | CCCCTGGCCA GAGAGTGGGA CTGCTGGGAA GAACAGGCTC TGGCAAGTCT ACCCTGCTGT |
| 4501 | CTGCCTTCCT GAGGCTGCTG AACACAGAGG GAGAGATCCA GATTGATGGA GTGTCCTGGG |
| 4561 | ACAGCATCAC ACTGCAGCAG TGGAGGAAGG CCTTTGGTGT GATCCCCCAG AAAGTGTTCA |
| 4621 | TCTTCAGTGG CACCTTCAGG AAGAACCTGG ACCCCTATGA GCAGTGGTCT GACCAGGAGA |
| 4681 | TTTGGAAAGT GGCTGATGAA GTGGGCCTGA GAAGTGTGAT TGAGCAGTTC CCTGGCAAGC |
| 4741 | TGGACTTTGT CCTGGTGGAT GGGGGCTGTG TGCTGAGCCA TGGCCACAAG CAGCTGATGT |
| 4801 | GCCTGGCCAG ATCAGTGCTG AGCAAGGCCA AGATCCTGCT GCTGGATGAG CCTTCTGCCC |
| 4861 | ACCTGGATCC TGTGACCTAC CAGATCATCA GGAGGACCCT CAAGCAGGCC TTTGCTGACT |
| 4921 | GCACAGTCAT CCTGTGTGAG CACAGGATTG AGGCCATGCT GGAGTGCCAG CAGTTCCTGG |
| 4981 | TGATTGAGGA GAACAAAGTG AGGCAGTATG ACAGCATCCA GAAGCTGCTG AATGAGAGGA |
| 5041 | GCCTGTTCAG GCAGGCCATC AGCCCCTCTG ATAGAGTGAA GCTGTTCCCC CACAGGAACA |
| 5101 | GCTCCAAGTG CAAGAGCAAG CCCCAGATTG CTGCCCTGAA GGAGGAGACA GAGGAGGAAG |
| 5161 | TGCAGGACAC CAGGCTGTGA GGGCCCTGTG CCTTCTAGTT GCCAGCCATC TGTTGTTTGC |
| 5221 | CCCTCCCCTG TGCCTTCCTT GACCCTGGAA GGTGCCACTC CCACTGTCCT TTCCTAATAA |
| 5281 | AATGAGGAAA TTGCATTGCA TTGTCTGAGT AGGTGTCATT CTATTCTGGG GGGTGGGGTG |
| 5341 | GGGCAGGACA GCAAGGGGGA GGATTGGGAA GACAATAGCA GGCATGCAGA TCAGCAGTTC |
| 5401 | AACCTGTTGA TAGTATGTAC TAAGCTCTCA TGTTTAATGT ACTAAGCTCT CATGTTTAAT |
| 5461 | GAACTAAACC CTCATGGCTA ATGTACTAAG CTCTCATGGC TAATGTACTA AGCTCTCATG |
| 5521 | TTTCATGTAC TAAGCTCTCA TGTTTGAACA ATAAAATTAA TATAAATCAG CAACTTAAAT |
| 5581 | AGCCTCTAAG GTTTTAAGTT TTATAAGAAA AAAAAGAATA TATAAGGCTT TTAAAGGTTT |
| 5641 | TAAGGTTTCC TAGGTTATCC TGGTACCTTA GAAAAACTCA TCCAGCATCA AATGAAACTG |
| 5701 | CAATTTATTC ATATCAGGAT TATCAATACC ATATTTTTGA AAAAGTCTTT TCTGTAATGA |
| 5761 | AGGAGAAAAC TCACCCAGGC AGTTCCATAG GATGGCAAGA TCCTGGTATC TGTCTGCAAT |
| 5821 | TCCAACTCTT CCAACATCAA TACAACCTAT TAATTTCCCC TCATCAAAAA TAAGGTTATC |
| 5881 | AAGTGAGAAA TCACCATGAG TGACCACTGA ATCTGGTGAG AATGGCAAAA GCTTATGCAT |
| 5941 | TTCTTTCCAG ACTTGTTCAA CAGGCCAGCC ATTTCTCTCA TCATCAAAAT CACTGGCATC |
| 6001 | AACCAAACCA TTATTCATTC TTGATTGGGC CTGAGCCAGT CTAAATACTC TATCAGAGTT |
| 6061 | AAAAGGACAA TTACAAACAG GAATGGAATG CAATCTTCTC AGGAACACTG CCAGGGCATC |
| 6121 | AACAATATTT TCACCTGAAT CAGGATATTC TTCTAATACC TGGAATGCTG TTTTCCCTGG |
| 6181 | GATGGCAGTG GTGAGTAACC ATGCATCATC AGGAGTTCTG ATAAAATGCT TGATGGTTGG |
| 6241 | AAGAGGCATA AATTCAGTCA GCCAGTTTAG TCTGACCATC TCATCTGTAA CATCATTGGC |
| 6301 | AACAGAACCT TTGCCATGTT TCAGAAACAA CTCTGGGGCA TCTGGCTTCC CATACAATCT |
| 6361 | ATAGATTGTG GCACCTGATT GCCCAACATT ATCTCTAGCC CATTTATACC CATATAAATC |
| 6421 | AGCATCCATG TTGGAATTTA ATCTTGGCCT GGAGCAAGAG GTTTCTCTTT GAATATGGCT |
| 6481 | CATGGATCCC CTCCTATAGT GAGTTGTATT ATACTATGCA GATATACTAT GCCAATGTTT |
| 6541 | AATTGTCAA |

1. A pharmaceutical composition, wherein the composition comprises from 1 ml to less than 10 mls of a complex of (i) a non-viral CpG dinucleotide-free plasmid comprising a nucleic acid encoding a CFTR polypeptide operatively linked to hCEF1 promoter, wherein the plasmid is at a concentration of 2 mg/ml to 3 mg/ml, and (ii) GL67A lipid mixture at a concentration of 10 mg/ml to 20 mg/ml; or wherein the composition is a lyophilized composition comprising (i) a non-viral CpG dinucleotide-free plasmid comprising a nucleic acid encoding a CFTR polypeptide operatively linked to hCEF I promoter and (ii) GL67A lipid mixture.

2. The pharmaceutical composition of claim 1 wherein the non-viral CpG dinucleotide-free plasmid is at a concentration of 2.28 mg/ml to 2.8 mg/ml, and (ii) the GL67A lipid mixture is at a concentration of 12.2 mg/ml to 16.4 mg/ml.

3. The pharmaceutical composition of claim 1 wherein the non-viral CpG dinucleotide-free plasmid is at a concentration of 2.65 mg/ml, and (ii) the GL67A lipid mixture is at a concentration of 14.31 mg/ml.

4. The composition of claim 1 wherein the non-viral CpG dinucleotide-free plasmid has a nucleic acid sequence at least 90% identical to the nucleic acid sequence provided in SEQ ID NO:1.

5. The composition of claim 1 wherein the composition comprises 5 mls of the non-viral CpG dinucleotide-free plasmid/GL67A lipid mixture complex.

6. The composition of claim 1 wherein the composition is formulated as an aerosol.

7. The composition of claim 6 wherein the aerosol has a droplet size having a Mass Median Aerodynamic Diameter (MMAD) of less than 5 μm, and having a Fine Particle Fraction greater than 50%; and having greater than 50% of the total aerosolizsed plasmid delivered intact.

8. The composition of claim 6 wherein the aerosol has a Mass Median Aerodynamic Diameter (MMAD) in the range 2-5₁ am and a Fine Particle Fraction (FPF) greater than 60%.

9. A method of treating cystic fibrosis comprising administering to a patient in need thereof a pharmaceutical composition of claim 1 prepared as a unit dose.

10. The method of claim 9 wherein the method comprises administering the composition as an aerosol.

11. The method of claim 10 wherein the method comprises administering the composition by a breath-actuated nebulizer having a formulation capacity of between 2 ml and 10 ml.

12. The method of claim 11 wherein the breath-actuated nebulizer is an AeroEclipse II nebulizer.

13. The method of claim 10 wherein the aerosol is administered to a patient at an aerosol delivery rate of between 80 μl/min and 400 μl/min.

14. The method of claim 10 wherein the method of treating cystic fibrosis comprises administering the composition in a cyclic treatment regimen in which the patient inhales the aerosol for a period of time followed by a rest period.

15. The method of claim 14 wherein the patient inhales the aerosol for 3 minutes followed by a 2 minute rest period.

16. The method of claim 9 wherein the method of treating cystic fibrosis comprises repeatedly administering the unit dose of the pharmaceutical composition to a patient in need thereof.

17. The method of claim 9 wherein the method of treating cystic fibrosis comprises repeatedly administering the composition to a patient in need thereof according to a continuous schedule having a once-monthly dosing interval.

18. The method of claim 9 further comprising an anti-pyretic agent.

19. A method for preparing a pharmaceutical composition for the treatment of cystic fibrosis, the method comprising (i) placing a quantity of a liquid composition of a non-viral CpG dinucleotide-free plasmid comprising nucleic acid encoding a CFTR polypeptide operatively linked to hCEF1 promoter in a first compartment; (ii) placing a quantity of a liquid composition of GL67A lipid mixture (ii) in a second compartment; (iii) co-extrusion of the contents of the first and second compartments through a static mixer at a fluidic flow rate having a laminar flow defined by a Reynolds number of less than or equal to 2300.

20. The method of claim 19 wherein the non-viral CpG dinucleotide-free plasmid is at a concentration of 1.5 mg/ml to 3.5 mg/ml and the GL67A lipid mixture is at a concentration of 10 mg/ml to 20 mg/ml.

21. The method of claim 19 wherein the compositions are mixed in a static mixer containing between 4 and 16 elements and having a diameter of between 1 mm and 5 mm.

22. (canceled)

23. The pharmaceutical composition of claim 1 wherein the composition is a lyophilized composition containing from 2 mg to 30 mg of the non-viral CpG dinucleotide-free plasmid and 10 mg to 200 mg of GL67A lipid mixture, or is a lyophilized composition containing from 2.65 mg to 26.5 mg of the non-viral CpG dinucleotide-free plasmid and 14.31 mg to 143.1 mg of the GL67A lipid mixture.

24. (canceled)

25. The pharmaceutical composition according to claim 1 wherein the composition is a lyophilize composition further comprising a cryoprotectant.

26. The pharmaceutical composition according to claim 1 wherein the composition is a lyophilized composition prepared as a dry powder formulation.

27-31. (canceled)
